(19) United States
(12) Patent Application Publication
(10) Pub. No.: US 2011/0250237 A1
(43) Pub. Date: Oct. 13, 2011

(54) IMMUNOGENIC AMPHIPATHIC PEPTIDE COMPOSITIONS

(76) Inventors: Derek O'Hagan, Winchester, MA (US); Andrew Geall, Littleton, MA (US); Philip Dormitzer, Weston, MA (US)

(21) Appl. No.: 13/003,557

(22) PCT Filed: Jul. 15, 2009
(86) PCT No.: PCT/US2009/050764

§ 371 Date: Jun. 26, 2011

Related U.S. Application Data
(60) Provisional application No. 61/080,942, filed on Jul. 15, 2008.

(51) Int. Cl. A61K 9/14; A61K 39/145; A61P 37/04; A61P 31/16; A61K 39/00; A61K 39/39; B82Y 5/00

(52) U.S. Cl. 424/400; 424/184.1; 424/210.1; 424/278.1; 424/282.1; 424/193.1; 977/773; 977/915

(57) ABSTRACT

The present application pertains to a composition, comprising (a) amphipathic peptides; (b) lipids and (c) at least one immunogenic species. Respective compositions are suitable for immunogenic species transport and delivery, for example for systemic or local delivery to a mammal. Also provided are pharmaceutical compositions, comprising respective compositions. Methods of forming the foregoing are also provided.

### FIELD OF THE INVENTION

The present technology pertains to amphipathic peptide compositions and their use for the delivery and transportation of immunogenic species.

### BACKGROUND OF THE INVENTION

Particulate carriers, including polymeric carriers, have been used with adsorbed or entrapped antigens and adjuvants in attempts to elicit adequate immune responses. Such particulate carriers present multiple copies of a selected antigen or adjuvant to the immune system and may promote trapping and retention in local lymph nodes. The particles can be phagocytosed by cells such as macrophages and can enhance antigen presentation through cytokine release.

### BRIEF SUMMARY OF THE INVENTION

The present invention is directed to immunogenic compositions comprising amphipathic peptides and lipids for the delivery of immunogenic species.

In one embodiment, the composition comprises amphipathic peptides, lipids and at least one immunogenic species.

In one embodiment, the immunogenic species is a species that stimulates an adaptive immune response. For example, the immunogenic species may comprise one or more antigens. Examples of antigens include polypeptide-containing antigens, polysaccharide-containing antigens, and polynucleotide-containing antigens, among others. Antigens can be derived, for example, from tumor cells and from pathogenic organisms such as viruses, bacteria, fungi and parasites, among other sources.

In one embodiment, the immunogenic species are species that stimulate an innate immune response. For example, the immunogenic species may be an activator of one or more of the following receptors, among others: Toll-like receptors (TLRs), nucleotide-binding oligomerization domain (NOD) proteins, and receptors that induce phagocytosis, such as scavenger receptors, mannose receptors and β-glucan receptors.

In one embodiment, the immunogenic species may be selected, for example, from one or more of the following immunological adjuvants: lipopolysaccharides including bacterial lipopolysaccharides, peptidoglycans, bacterial lipoproteins, bacterial flagellins, imidazoquinoline compounds, lipopeptides, benzonaphthyridine compounds, immunostimulatory oligonucleotides, single-stranded RNA, saponins, lipoteichoic acid, ADP-ribosylating toxins and detoxified derivatives thereof, polyphosphazene, muramyl peptides, thiosemicarbazone compounds, tryptanthrin compounds, and lipid A derivatives, among many others.

The present invention further embodies particles comprising amphipathic peptides and lipids employed as a carrier for the delivery of at least one immunogenic species, as well as aggregates of such particles.

The present invention also provides a method for producing a composition comprising amphipathic peptides, lipids and at least one immunogenic species, wherein the lipids, amphipathic peptides and immunogenic species are mixed and processed to form particles. For example, a solution comprising a mixture the lipids, amphipathic peptides and immunogenic species may be cast into a film, and the film may be rehydrdated to form such particles.

The present invention also provides a method for producing a composition comprising amphipathic peptides, lipids and at least one immunogenic species, wherein the lipids are mixed with the amphipathic peptides and processed to form particles and the particles are contacted with at least one immunogenic species.

The present invention also provides a method for producing a composition comprising amphipathic peptides, lipids and at least one immunogenic species, wherein the lipids are mixed with the amphipathic peptides and processed to form particles and the at least one immunogenic species is formed or modified in the presence of such particles.

For example, in some embodiments, an immunogenic protein may be formed from amino acids in the presence of such particles. In some embodiments, an immunogenic protein may be modified in the presence of such particles, for instance, by cleaving an immunogenic protein to render it more hydrophobic and/or to expose a hydrophobic portion of the immunogenic protein.

Other objects, features, advantages, embodiments, and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following.

### BRIEF DESCRIPTION OF THE DRAWINGS

FIG. 1 shows an overlay of size exclusion chromatograms of particles made at a peptide to lipid molar ratio of 1:1.75, 1:3 and 1:7 and peptide alone at a concentration of 2 mg/ml using peptide of SEQ ID NO:1 and lipid POPC. About 200 μl of particles in normal saline were injected onto a superpose 6 column using 50 mM sodium phosphate with 150 mM sodium chloride at 0.5 ml/min as elution buffer with run times up to 40 ml elution volumes. The chromatograms shows UV absorbance at 215 nm plotted against elution volume. FIG. 1 demonstrates that the size of the particles varies, depending on the used peptide to lipid molar ratio. An increase in lipids relative to peptide renders bigger particles. Of the peptide to lipid molar ratios tested, 1:1.75 provided the smallest particles that had a size of less than 10 nm. A respective size exclusion chromatogram also allows the determination of the Stokes diameter, when an appropriate standard is used. An alternative method for determining the diameter of the particles is dynamic light scattering and the same trends are observed.

FIG. 2 shows a size exclusion chromatogram of particles made using peptide SEQ ID NO: 1 and lipid POPC at peptide to lipid molar ratio of 1:1.75. About 200 μl of particles at a peptide concentration of 8 mg/ml were injected onto a superpose 6 column using 50 mM sodium phosphate with 150 mM sodium chloride at 0.5 ml/min as elution buffer with run times up to 40 ml elution volumes. Fractions of 0.5 ml each were collected in a 96-well plate in series of rows throughout the run and are plotted along with the elution volumes against absorbance at 215 nm on the chromatogram. The fractions from C₉-D₉ were pooled and concentrated by tangential flow filtration using MicroKros hollow fibers (Spectrum Labs) made of polysulfone with 50 KD cut-off.

FIG. 3 shows 2-dimensional NMR spectra (NOESY Spectra) of particles made at a peptide to lipid molar ratio of 1:1.75 in 5 mM potassium phosphate (KH₂PO₄) buffer made in 90% v/v H₂O and 10% v/v D₂O at pH 6.23, 37° C. The particles with peptide at a concentration of 2 mg/ml of peptide SEQ ID NO:.1 were used to collect data on Bruker-Biospin NMR at 600 MHz. FIG. 5 shows an enlarged section of FIG. 3 (of the left upper corner).

FIG. 4 shows the structure and proton assignment of the lipid POPC by nuclear magnetic resonance (NMR). For this purpose, a lipid film is made by evaporating off excess methanol from the stock solution of POPC in methanol. A lipid solution or liposomes of POPC were made by hydrating the lipid film with deuterated methylene chloride at a concentration of 1 mg/ml.

FIG. 5 shows 2-dimensional NMR spectra (NOESY Spectra) of particles made using peptide Seq ID No.1 and lipid POPC. The picture is an enlarged view of the left upper corner from FIG. 3. The x-dimension (6-9 ppm) represents proton signals of aromatic amino acids and the y-dimension (0-5 ppm) represents proton signals of lipid and side chains of aromatic amino acids. The particles used were made at a peptide to lipid molar ratio of 1:1.75 in 5 mM potassium phosphate (KH₂PO₄) buffer made in 90% v/v H₂O and 10% v/v D₂O at pH 6.23, 37° C. The particles with peptide at a concentration of 2 mg/ml were used to collect data on Bruker-Biospin NMR at 600 MHz.

In sum, FIGS. 3 to 5 demonstrate that the peptides have a helical structure in the particles according to the present invention, that the helical peptides interact with the lipids on a molecular level at a defined space and that the particles have a defined structure.

FIGS. 6A-6C show size exclusion chromatograms of particles along with human lipoproteins. The particles with peptide Seq ID No.1 and lipid POPC were used at a peptide to lipid molar ratio of 1:1.75. The chromatograms show injection overlay of (a) particles at a peptide concentration of 2 mg/ml, high density lipoproteins (HDL) at 1 mg/ml and a mixture of HDL and particles (0.5 and 1 mg/ml respectively), (b) particles at a peptide concentration of 2 mg/ml, low density lipoproteins (LDL) at 1 mg/ml and a mixture of LDL and particles (0.5 and 1 mg/ml respectively), and (c) particles at a peptide concentration of 2 mg/ml, very low density lipoproteins (VLDL) at 0.877 mg/ml and a mixture of VLDL and particles (0.438 and 1 mg/ml respectively). FIGS. 6A-6C show again the remarkable stability of the particles according to the present application. Even when mixing the particles according to the present invention (NLPP—Nano Lipid Peptide Particles) with natural lipoproteins such as HDL, LDL and VLDL, and thus with natural lipoproteins, the NLPPs remain as a distinct fraction. Hence, the particles do not interact with the natural lipoproteins or form aggregates or disintegrate under the tested conditions. Accordingly, they are stable in the presence of other lipoproteins. This is an important characteristic for pharmaceutical applications and it also enables efficient targeting.

FIGS. 7A-7B show differential scanning calorimetry of peptide and particles.

FIGS. 8A-8C, 9A-9B and 10 schematically illustrate various ways to combine the desired elements, in order to attach the immunogenic species to the particles formed of the amphipathic peptides and lipids.

FIG. 11 schematically shows an embodiment for functionalizing the amphipathic peptides of the invention. An amphipathic peptide is shown, wherein the lysine side chains are available and thus accessible for chemical modification. The lysine side chains are modified with an alkyne and thus provide an anchoring site for attaching a targeting ligand TL.

FIG. 12 shows various lipidated targeting motifs useful for particle targeting.

FIG. 13 illustrates stimulation of HEK293-NF-κBluc-FLAGTLR2 cells by different forms of empty NLPP (without lipopeptide), by PAM₃CSK₄ and by sonicated lipopeptide.

FIG. 14 illustrates stimulation of HEK293-NF-κBluc-FLAGTLR2 cells by NLPP containing lipopeptide, by PAM₃CSK₄ and by sonicated lipopeptide.

FIG. 15 shows size exclusion chromatograms of NLPP particles, using the e2695 Separations Module method.

FIG. 16 shows a size exclusion chromatograms of NLPP particles, using the Akta Explorer 900 method.

FIG. 17 illustrates: (a) size exclusion chromatogram for NLPP particles at a lipid:DMPC ratio of 1:2.5 and containing SMIP at a concentration of 1.2 mg/mL and (b) size exclusion chromatography fraction analysis for SMIP and phospholipid content.

FIG. 18 is a schematic of RSV F protein showing the signal sequence or signal peptide (SP), p27 linker region, fusion peptide (FP), HRA domain (HRA), HRB domain (HRB), transmembrane region (TM), and cytoplasmic tail (CT). Furin cleavage site are present at amino acid positions 109 and 136. FIG. 18 also shows the amino acid sequence of amino acids 100-150 of RSV F (wild type) (SEQ ID NO: 6) and a protein in which the furin cleavage sites were mutated (SEQ ID NO: 7). In FIG. 18, the symbol “-” indicates that the amino acid at that position is deleted.

FIG. 19 is a plot of IL-6 production by human PBMC upon stimulation by different forms of empty NLPP (without LIPO1).

FIG. 20 is a plot of IL-8 production by mouse splenocytes upon stimulation by different forms of empty NLPP (without LIPO1).

FIG. 21 is a plot of IL-6 production by human PBMC upon stimulation by NLPP containing Lipo 1 lipopeptide.

FIG. 22 is a plot of IL-8 production by mouse splenocytes upon stimulation by NLPP containing Lipo 1 lipopeptide.

### DETAILED DESCRIPTION OF THE INVENTION

### Definitions

The term “alkenyl,” as used herein, refers to a partially unsaturated branched or straight chain hydrocarbon having at least one carbon-carbon double bond. Atoms oriented about the double bond are in either the cis (Z) or trans (E) conformation. An alkenyl group can be optionally substituted. As used herein, the terms “C₂-C₃alkenyl”, “C₂-C₄alkenyl”, “C₂-C₅alkenyl”, “C₂-C₆alkenyl”, “C₂-C₇alkenyl”, and “C₂-C₈alkenyl” refer to an alkenyl group containing at least 2, and at most 3, 4, 5, 6, 7 or 8 carbon atoms, respectively. If not otherwise specified, an alkenyl group generally is a C₂-C₆ alkenyl. Non-limiting examples of alkenyl groups, as used herein, include ethenyl, propenyl, butenyl, pentenyl, hexenyl, heptenyl, octenyl, nonenyl, decenyl and the like.

The term “alkenylene,” as used herein, refers to a partially unsaturated branched or straight chain divalent hydrocarbon radical derived from an alkenyl group. An alkenylene group can be optionally substituted. As used herein, the terms “C₂-C₃alkenylene”, “C₂-C₄alkenylene”, “C₂-C₅alkenylene”, “C₂-C₆alkenylene”, “C₂-C₇alkenylene”, and “C₂-C₈alkenylene” refer to an alkenylene group containing at least 2, and at most 3, 4, 5, 6, 7 or 8 carbon atoms respectively. If not otherwise specified, an alkenylene group generally is a C₁-C₆ alkenylene. Non-limiting examples of alkenylene groups as used herein include, ethenylene, propenylene, butenylene, pentenylene, hexenylene, heptenylene, octenylene, nonenylene, decenylene and the like.

The term “alkyl,” as used herein, refers to a saturated branched or straight chain hydrocarbon. An alkyl group can be optionally substituted. As used herein, the terms “C₁-C₃alkyl”, “C₁-C₄alkyl”, “C₁-C₅alkyl”, “C₁-C₆alkyl”, “C₁-C₇alkyl” and “C₁-C₈alkyl” refer to an alkyl group containing at least 1, and at most 3, 4, 5, 6, 7 or 8 carbon atoms, respectively. If not otherwise specified, an alkyl group generally is a C₁-C₆ alkyl. Non-limiting examples of alkyl groups as used herein include methyl, ethyl, n-propyl, isopropyl, n-butyl, isobutyl, sec-butyl, t-butyl, n-pentyl, isopentyl, hexyl, heptyl, octyl, nonyl, decyl and the like.

The term “alkylene,” as used herein, refers to a saturated branched or straight chain divalent hydrocarbon radical derived from an alkyl group. An alkylene group can be optionally substituted. As used herein, the terms “C₁-C₃alkylene”, “C₁-C₄alkylene”, “C₁-C₅alkylene”, “C₁-C₆alkylene”, “C₁-C₇alkylene” and “C₁-C₈alkylene” refer to an alkylene group containing at least 1, and at most 3, 4, 5, 6, 7 or 8 carbon atoms respectively. If not otherwise specified, an alkylene group generally is a C₁-C₆ alkylene. Non-limiting examples of alkylene groups as used herein include, methylene, ethylene, n-propylene, isopropylene, n-butylene, isobutylene, sec-butylene, t-butylene, n-pentylene, isopentylene, hexylene and the like.

The term “alkynyl,” as used herein, refers to a partially unsaturated branched or straight chain hydrocarbon having at least one carbon-carbon triple bond. An alkynyl group can be optionally substituted. As used herein, the terms “C₂-C₃alkynyl”, “C₂-C₄alkynyl”, “C₂-C₅alkynyl”, “C₂-C₆alkynyl”, “C₂-C₇alkynyl”, and “C₂-C₈alkynyl” refer to an alkynyl group containing at least 2, and at most 3, 4, 5, 6, 7 or 8 carbon atoms, respectively. If not otherwise specified, an alkynyl group generally is a C₂-C₆ alkynyl. Non-limiting examples of alkynyl groups, as used herein, include ethynyl, propynyl, butynyl, pentynyl, hexynyl, heptynyl, octynyl, nonynyl, decynyl and the like.

The term “alkynylene,” as used herein, refers to a partially unsaturated branched or straight chain divalent hydrocarbon radical derived from an alkynyl group. An alkynylene group can be optionally substituted. As used herein, the terms “C₂-C₃alkynylene”, “C₂-C₄alkynylene”, “C₂-C₅alkynylene”, “C₂-C₆alkynylene”, “C₂-C₇alkynylene”, and “C₂-C₈alkynylene” refer to an alkynylene group containing at least 2, and at most 3, 4, 5, 6, 7 or 8 carbon atoms respectively. If not otherwise specified, an alkynylene group generally is a C₂-C₆ alkynylene. Non-limiting examples of alkynylene groups as used herein include, ethynylene, propynylene, butynylene, pentynylene, hexynylene, heptynylene, octynylene, nonynylene, decynylene and the like.

The term “alkoxy,” as used herein, refers to the group —ORₐ, where Rₐ is an alkyl group as defined herein. An alkoxy group can be optionally substituted. As used herein, the terms “C₁-C₃alkoxy”, “C₁-C₄alkoxy”, “C₁-C₅alkoxy”, “C₁-C₆alkoxy”, “C₁-C₇alkoxy” and “C₁-C₈alkoxy” refer to an alkoxy group wherein the alkyl moiety contains at least 1, and at most 3, 4, 5, 6, 7 or 8, carbon atoms. Non-limiting examples of alkoxy groups, as used herein, include methoxy, ethoxy, n-propoxy, isopropoxy, n-butyloxy, t-butyloxy, pentyloxy, hexyloxy, heptyloxy, octyloxy, nonyloxy, decyloxy and the like.

The term “aryl,” as used herein, refers to monocyclic, bicyclic, and tricyclic ring systems having a total of five to fourteen ring members, wherein at least one ring in the system is aromatic and wherein each ring in the system contains 3 to 7 ring members. An aryl group can be optionally substituted. Non-limiting examples of aryl groups, as used herein, include phenyl, naphthyl, fluorenyl, indenyl, azulenyl, anthracenyl and the like.

The term “arylene,” as used means a divalent radical derived from an aryl group. An arylene group can be optionally substituted.

The term “cyano,” as used herein, refers to a —CN group.

The term “cycloalkyl,” as used herein, refers to a saturated or partially unsaturated, monocyclic, fused bicyclic, fused tricyclic or bridged polycyclic ring assembly. As used herein, the terms “C₃-C₅ cycloalkyl”, “C₃-C₆ cycloalkyl”, “C₃-C₇ cycloalkyl”, “C₃-C₈ cycloalkyl, “C₃-C₉ cycloalkyl and “C₃-C₁₀ cycloalkyl refer to a cycloalkyl group wherein the saturated or partially unsaturated, monocyclic, fused bicyclic or bridged polycyclic ring assembly contain at least 3, and at most 5, 6, 7, 8, 9 or 10, carbon atoms. A cycloalkyl group can be optionally substituted. Non-limiting examples of cycloalkyl groups, as used herein, include cyclopropyl, cyclobutyl, cyclopentyl, cyclohexyl, cycloheptyl, cyclooctyl, cyclononyl, cyclodecyl, cyclopentenyl, cyclohexenyl, decahydronaphthalenyl, 2,3,4,5,6,7-hexahydro-1H-indenyl and the like.

The term “halogen,” as used herein, refers to fluorine (F), chlorine (Cl), bromine (Br), or iodine (I).

The term “halo,” as used herein, refers to the halogen radicals: fluoro (—F), chloro (—Cl), bromo (—Br), and iodo (—I).

The terms “haloalkyl” or “halo-substituted alkyl,” as used herein, refers to an alkyl group as defined herein, substituted with one or more halogen groups, wherein the halogen groups are the same or different. A haloalkyl group can be optionally substituted. Non-limiting examples of such branched or straight chained haloalkyl groups, as used herein, include methyl, ethyl, propyl, isopropyl, isobutyl and n-butyl substituted with one or more halogen groups, wherein the halogen groups are the same or different, including, but not limited to, trifluoromethyl, pentafluoroethyl, and the like.

The terms “haloalkenyl” or “halo-substituted alkenyl,” as used herein, refers to an alkenyl group as defined herein, substituted with one or more halogen groups, wherein the halogen groups are the same or different. A haloalkenyl group can be optionally substituted. Non-limiting examples of such branched or straight chained haloalkenyl groups, as used herein, include ethenyl, propenyl, butenyl, pentenyl, hexenyl, heptenyl, octenyl, nonenyl, decenyl and the like substituted with one or more halogen groups, wherein the halogen groups are the same or different.

The terms “haloalkynyl” or “halo-substituted alkynyl,” as used herein, refers to an alkynyl group as defined above, substituted with one or more halogen groups, wherein the halogen groups are the same or different. A haloalkynyl group can be optionally substituted. Non-limiting examples of such branched or straight chained haloalkynyl groups, as used herein, include ethynyl, propynyl, butynyl, pentynyl, hexynyl, heptynyl, octynyl, nonynyl, decynyl, and the like substituted with one or more halogen groups, wherein the halogen groups are the same or different.

The term “haloalkoxy,” as used herein, refers to an alkoxy group as defined herein, substituted with one or more halogen groups, wherein the halogen groups are the same or different. A haloalkoxy group can be optionally substituted. Non-limiting examples of such branched or straight chained haloalkynyl groups, as used herein, include methoxy, ethoxy, n-propoxy, isopropoxy, n-butyloxy, t-butyloxy, pentyloxy, hexyloxy, heptyloxy, octyloxy, nonyloxy, decyloxy and the like, substituted with one or more halogen groups, wherein the halogen groups are the same or different.

The term “heteroalkyl,” as used herein, refers to an alkyl group as defined herein wherein one or more carbon atoms are independently replaced by one or more of oxygen, sulfur, nitrogen, or combinations thereof.

The term “heteroaryl,” as used herein, refers to monocyclic, bicyclic, and tricyclic ring systems having a total of five to fourteen ring members, wherein at least one ring in the system is aromatic, at least one ring in the system contains one or more heteroatoms selected from nitrogen, oxygen and sulfur, and wherein each ring in the system contains 3 to 7 ring members. A heteroaryl group may contain one or more substituents. A heteroaryl group can be optionally substituted. Non-limiting examples of heteroaryl groups, as used herein, include benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridyl, pyridazinyl, pyrazinyl, pyrimidinyl, quinoxalinyl, quinolinyl, quinazolinyl, 4H-quinolizinyl, thiazolyl, thiadiazolyl, thienyl, triazinyl, triazolyl and tetrazolyl.

The term “heterocycloalkyl,” as used herein, refers to a cycloalkyl, as defined herein, wherein one or more of the ring carbons are replaced by a moiety selected from —O—, —N═, —NR—, —C(O)—, —S—, —S(O)— or —S(O)₂—, wherein R is hydrogen, C₁-C₄alkyl or a nitrogen protecting group, with the proviso that the ring of said group does not contain two adjacent O or S atoms. A heterocycloalkyl group can be optionally substituted. Non-limiting examples of heterocycloalkyl groups, as used herein, include morpholino, pyrrolidinyl, pyrrolidinyl-2-one, piperazinyl, piperidinyl, piperidinylone, 1,4-dioxa-8-aza-spiro[4.5]dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo[3.1.0]hexanyl, and 3-azabicyclo[4.1.0]heptanyl.

The term “heteroatom,” as used herein, refers to one or more of oxygen, sulfur, nitrogen, phosphorus, or silicon.

The term “hydroxyl,” as used herein, refers to the group —OH.

The term “hydroxyalkyl,” as used herein refers to an alkyl group as defined herein substituted with one or more hydroxyl group. Non-limiting examples of branched or straight chained “C₁-C₆ hydroxyalkyl groups as used herein include methyl, ethyl, propyl, isopropyl, isobutyl and n-butyl groups substituted with one or more hydroxyl groups.

The term “isocyanato,” as used herein, refers to a N═C═O group.

The term “isothiocyanato,” as used herein, refers to a —N═C═S group

The term “mercaptyl,” as used herein, refers to an (alkyl)S— group.

The term “optionally substituted,” as used herein, means that the referenced group may or may not be substituted with one or more additional group(s) individually and independently selected from alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and di-substituted amino groups, and the protected derivatives thereof. Non-limiting examples of optional substituents include, halo, —CN, ═O, ═N—OH, ═N—OR, ═N—R, —OR, —C(O)R, —C(O)OR, —OC(O)R, —OC(O)OR, —C(O)NHR, —C(O)NR₂, —OC(O)NHR, —OC(O)NR₂, —SR—, —S(O)R, —S(O)₂R, —NHR, —N(R)₂, —NHC(O)R, —NRC(O)R, —NHC(O)OR, —NRC(O)OR, S(O)₂NHR, —S(O)₂N(R)₂, —NHS(O)₂NR₂, —NRS(O)₂NR₂, —NHS(O)₂R, —NRS(O)₂R, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy, where each R is independently selected from H, halo, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy. The placement and number of such substituent groups is done in accordance with the well-understood valence limitations of each group, for example ═O is a suitable substituent for an alkyl group but not for an aryl group.

The term “prodrug,” as used herein, refers to an agent that is converted into the parent drug in vivo. A non-limiting example of a prodrug of the compounds described herein is a compound described herein administered as an ester which is then metabolically hydrolyzed to a carboxylic acid, the active entity, once inside the cell. A further example of a prodrug is a short peptide bonded to an acid group where the peptide is metabolized to reveal the active moiety.

The term “solvate,” as used herein, refers to a complex of variable stoichiometry formed by a solute (by way of example, a compound of Formula (I), or a salt thereof, as described herein) and a solvent. Non-limiting examples of a solvent are water, acetone, methanol, ethanol and acetic acid.

According to one embodiment of the present application, a composition is provided, comprising: (a) amphipathic peptides; (b) lipids; and (c) at least one immunogenic species.

### A. Amphipathic Peptides and Lipids

The amphipathic peptides used for creating the compositions of the present application may be of the same kind or may comprise peptides of a different kind, e.g. of a different amino acid sequence. The peptides can be composed of L and/or D amino acids and may comprise natural as well as non-natural amino acids and amino acid analogues. The peptides may have an amino acid chain length of less than or equal to 100, 50, 35, 30, 25 or less than or equal to 20 amino acids. In certain preferred embodiments, the peptides have an amino acid chain length of less than or equal to 30, 25 or 20 amino acids. Such short chain peptides may be desirable from an immunological standpoint, because they may provoke a diminished adaptive immune response (or none at all) relative to longer chain amphipathic peptides Immune responses may also be diminished/eliminated by forming the amphipathic peptides from non-natural amino acids such as 3-iodo-L-tyrosine or 5-hydroxy-tryptophan, among others.

The amphipathic peptides used according to the teaching of the present application solubilize the lipids and form particles. The thus formed particles may comprise a core of lipids, wherein the helices of the amphipathic peptides are assembled around the lipid core (e.g., in a belt-like fashion), thereby shielding the hydrophobic parts of the lipids. The shape of the particles may resemble a disc. As amphipathic peptides and lipids for respective particles can be synthetically produced, they may be of a defined composition and size. They can also be scaled up to large quantities and can be produced with a uniform size, thereby depicting significant advantages over natural lipoproteins. Furthermore, it has been shown in extensive experiments that the particles according to the present application are remarkably stable and can be purified and processed (e.g. sterile filtered). These are important advantages for an industrial production process. Furthermore, it was shown that the particles according to the present application are also stable in the presence of natural lipoproteins such as HDL, VLDL or LDL. This is an important advantage for in vivo applications as undesired aggregations or interactions with natural lipoproteins are avoided.

The respective particles comprising amphipathic peptides and lipids can be loaded with at least one immunogenic species in order to form immunogenic compositions. Hence, the respective particles are suitable as carriers/vehicles for delivery of immunogenic species. Respective compositions are in particular suitable for delivering at least one immunogenic species to a recipient, e.g. a vertebrate subject (i.e., any member of the subphylum cordata, including, without limitation, mammals such as cattle, sheep, pigs, goats, horses, and humans; domestic animals such as dogs and cats; and birds, including domestic, wild and game birds such as cocks and hens including chickens, turkeys and other gallinaceous birds) and in particular a human. The respective particles formed have a synthetic structure similar to that of known lipoproteins, such as HDL. An important advantage over natural HDL is that the particles according to the present application can be synthetically produced at a large scale. They have a defined composition and are also stable under various conditions. Their defined composition and stability profile make them particularly suitable for pharmaceutical applications.

It was found advantageous to use an amphipathic peptide that is capable of mimicking one or more properties of apolipoprotein A1. Apolipoproteins are lipid-binding proteins that are divided into 6 major classes (A, B, C, D, E and H) and several sub-classes. Apolipoproteins in lipoproteins are classified into exchangeable (apo A-I, A-II, A-IV, C-I, C-II, C-III and E) and non-exchangeable apolipoproteins (apo B-100 and B-48). They are synthesized in the liver and intestine. The exchangeable apolipoproteins are capable of exchange between different lipoprotein particles during lipid metabolism. Structurally, these exchangeable apolipoproteins contain different classes of amphipathic helices-class A (sub-classes A1, A2 and A4), class Y and class G, which impart lipid affinity to apolipoproteins.

An amphipathic helix contains hydrophilic amino acids on the polar face and hydrophobic amino acids on the non-polar face. The distribution and clustering of charged amino acid residues in the polar face of the helix is the predominant difference among different classes of amphipathic helices. The design and synthesis of respective peptides that are capable of mimicking the properties of apolipoprotein A1 is known in the prior art, please refer for example to Mishra et al. “Interaction of Model Class A1, Class A2, and Class Y Amphipathic Helical Peptides with Membranes”, Biochemistry 1996, Aug. 27; 35(34):11210-20, herein incorporated fully by reference. Furthermore, respective synthetic peptide analogs are known which are able to mimic the lipid-binding and Lecithin-Cholesterol Acetyltransferase (LCAT) activation properties of apolipoproteins. Amphipathic peptides of varying lengths have been designed by various researchers for optimum alpha helicity, lipid-binding and LCAT activation.

Suitable amphipathic peptides that can be used according to the present invention are, for example, described in Mishra V K, Anantharamaiah G M, Segrest J P, Palgunachari M N, Chaddha M, Sham S W, Krishna N R. “Association of a model class A (apolipoprotein) amphipathic alpha helical peptide with lipid: high resolution NMR studies of peptide lipid discoidal complexes.” J Biol. Chem. 2006 Mar. 10; 281(10):6511-9; Mishra V K, Palgunachari M N. “Interaction of model class A1, class A2, and class Y amphipathic helical peptides with membranes.” Biochemistry. 1996 Aug. 27; 35(34):11210-20; Anantharamaiah G M. “Synthetic peptide analogs of apolipoproteins.” Methods Enzymol. 1986; 128:627-47; Navab M, Anantharamaiah G M, Reddy S T, Hama S, Hough G, Grijalva V R, Yu N, Ansell B J, Datta G, Garber D W, Fogelman A M. “Apolipoprotein A-I mimetic peptides.” Arterioscler Thromb Vasc Biol. 2005 July; 25(7):1325-31; Navab et al. “Apolipoprotein A-I mimetic peptides and their role in athereosclerosis prevention” Nature Clinical Practice October 2006 Vol 3 No. 10; herein incorporated by reference.

According to one embodiment, the amphipathic peptide used in the composition according to the present invention forms a class A amphipathic alpha helix.

The amphipathic peptides used according to the present application can be selected from a group of peptides comprising the following amino acid sequences:

| | | |
|---|---|---|
| i. | DWLKAFYDKVAEKLKEAFLA | (SEQ ID NO: 1) |
| ii. | ELLEKWKEALAALAEKLK | (SEQ ID NO: 2) |
| iii. | FWLKAFYDKVAEKLKEAF | (SEQ ID NO: 3) |
| iv. | DWLKAFYDKVAEKLKEAFRLTRKRGLKLA | (SEQ ID NO: 4) |
| v. | DWLKAFYDKVAEKLKEAF; | (SEQ ID NO: 5) |

vi. Functional analogs or fragments of the peptides according to i to v, capable of forming a class A amphipathic alpha helix.

Particularly advantageous peptides are peptides comprising or consisting of SEQ ID NO: 5 or SEQ. ID. NO: 1.

Peptide mimetics of apo A-1 commonly do not show any sequence homology to that of apo A-1 but are capable of forming a class A amphipathic alpha helix similar to apo A-1 and also show lipoprotein binding properties similar to that of apolipoproteins. The respective peptides have the ability to solubilize lipids and form particles with the lipids. According to one embodiment the amphipathic peptides show no sequence homology to apo A-1 or other lipoproteins.

According to one embodiment, at least one of the end groups of the peptides is blocked (i.e., either the N terminus, the C terminus, or both is blocked). For example, at least one of the termini may be acetylated and/or amidated. It was shown, that blocking at least one end group may increase the helical content of the peptide by removing the stabilizing interactions of the helix macrodipole with the charged termini. According to one embodiment, the N-terminal end is acetylated and the C-terminal end is amidated. According to one embodiment, the peptide Ac-Seq. ID. No. 1-NH₂ or Ac-Seq. ID. No. 5-NH₂ is used.

Furthermore, the peptides can be chemically modified in other ways, for example, in order to alter the physical and/or chemical properties of the particles. Such modifications can be done, for example, in order to target the particles, to increase their stability, to visualize them in vitro or in vivo, or to alter their distribution patterns, among other effects. Such modifications can be used alone or in combination with one or more other modifications to achieve the desired effects. Examples of modifications include but are not limited to biotinylation, fluorination and the conjugation of binding molecules such as antibodies or fragments thereof.

Modifying groups/species can be attached, for example, to either the C or N terminus or along the length of the peptides (e.g., attached to the side groups of the amino acids, for instance, side —NH₂ groups of lysine and arginine, side —COOH groups of glutamic acid and aspartic acid, etc.) with or without a linker of various lengths and compositions. It is also within the scope of the present application to modify appropriate side groups of the amino acids. Such modifying groups could be composed of small molecules, peptides, carbohydrates, antibodies or fragments thereof, aptameres, polymers or other molecular architectures.

The introduction of these modifications can be made by any method known in the prior art. For example, in addition to attachment to previously formed peptide chains, modified amino acids could be used in the synthesis of the peptide chain. Such unnatural amino acids could contain the entire desired modification, or a functionality such as a free or protected thiol group for use in forming disulphide bonds or adding into unsaturated systems, an azide or alkyne for use in cycloaddition chemistry (e.g., via azide-alkyne Huisgen cycloaddition, which is a 1,3-dipolar cycloaddition between an azide and a terminal or internal alkyne to give a 1,2,3-triazole), or an additional amino or carbonyl group for use in condensation reactions any of which could be used to introduce an extra functionality later in the synthesis.

The modifications may also be made to the peptides to increase the stability or improve the physical properties of the particles. Such modifications can include, but are not limited to, multimerisation of the peptide motifs by linking one of the ends of two or more peptides together, for cross-linking of peptides by linking side groups of natural or non-natural amino acids of one or more peptides together. Such connections can be made by linkers of various lengths and compositions. Multimerisation of the peptides can be accomplished as part of the peptide synthesis or by reacting functional groups on the peptide, such as amino side groups (e.g. of lysine), acid side groups (e.g., of glutamic acid), amino termini, acid termini, or unnatural amino acids comprising an appropriate functionality, with bifunctional or multi-functional linkers such as activated diacids, diamines or other compatible functional groups.

The lipids used in the present invention can also be of the same or different kind. The lipid that is used in the composition of the present application may have at least one of the following characteristics: (1) it is selected from the group consisting of triglycerides, phospholipids, cholesterol esters and cholesterol; (2) it is a neutral lipid; (3) it is a phospholipid; and/or (4) it is selected from the group of zwitterionic phospholipids consisting of phosphatidylcholines such as palmitoyl oleoyl phosphatidylcholine (POPC),

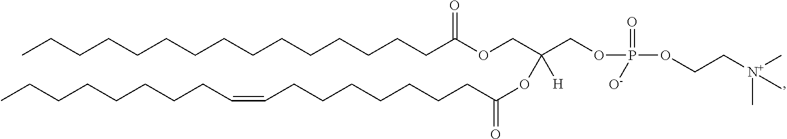

dimyristoyl phosphatidylcholine (DMPC),

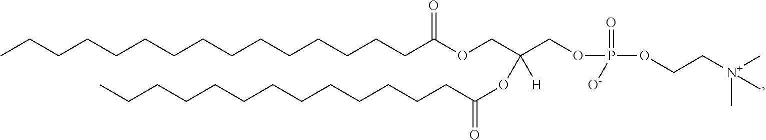

dioleoyl phosphatidylcholine (DOPC),

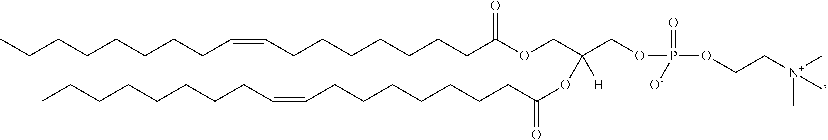

dipalmitoyl phosphatidylcholine (DPPC),

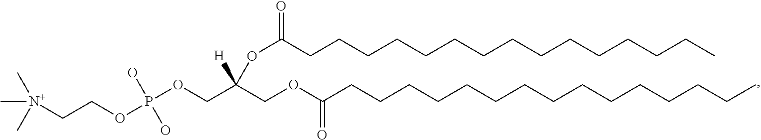

and palmitoyl linoleyl phosphatidylcholine (PLPC) as well as sphingomyelin.

The lipid may be selected from the group consisting of triglycerides, phospholipids, cholesterol esters and cholesterol. The respective lipids may also be selected from those found in lipoproteins of the human plasma. Lipoproteins may be divided into four major classes—chylomicrons, very low density lipoproteins, low density lipoproteins and high density proteins, which vary in size and compositions. Triglycerides, phospholipids, cholesterol esters and cholesterol are the major lipids present in the respective lipoproteins. It may be advantageous to use endogenous lipids in order to reduce the toxicity.

It may be advantageous to use a neutral lipid. It may also be advantageous to use a phospholipid, including a zwitterionic phospholipid, for example, a phospholipid containing one or more alkyl or alkenyl radicals of 12 to 22 carbons in length (e.g., 12 to 14 to 16 to 18 to 20 to 22 carbons), which radicals may contain, for example, from 0 to 1 to 2 to 3 double bonds. It may be advantageous to use a zwitterionic phospholipid. The lipid may be selected from the group consisting of POPC, DMPC, DOPC, DPPC, PLPC and sphingomyelin. Also other lipids can be used as long as they are able to form particles with the amphipathic peptides.

According to one embodiment, approximately 16 amphipathic peptides per particle form a double band around the lipid core comprising approximately 54 lipids. Of course, depending on the components and size of the particles, the amounts may vary.

According to one embodiment, the peptide to lipid molar ratio lies between 1:1 and 1:10 and more advantageously between 1:1.75 to 1:7. It was found that the size of the particles varies depending on the chosen peptide to lipid molar ratio. The more lipids that are used relative to peptide, the bigger the particles get. For obtaining rather small particles having a size of less than about 25 nm it is advantageous to use a peptide to lipid molar ratio of less than 1:2. Particularly advantageous results were achieved with a ratio of about 1:1.75.

It is advantageous that the particles formed by the amphipathic peptides and lipids, which may either be monodispersed or in the form of particle aggregates, have a size (i.e., width) of ranging from 3 nm or less to 5 nm to 10 nm to 20 nm to 25 nm to 30 nm to 35 nm to 50 nm to 100 nm to 250 nm to 500 nm to 1000 nm or more.

For certain applications it may be advantageous that the particles have a size of less than 50 nm, 35 nm, 30 nm, 25 nm, 20 nm, 10 nm, 5 nm or even less than 3 nm. To use rather small particles having a size of less than 25 nm or even less than 10 nm may be advantageous as they show a good systematic distribution to all body compartments and thus may also facilitate a target-specific uptake in some embodiments. The respective particles can be loaded with the at least one immunogenic species to be delivered. To use rather small particles is also advantageous in case targeting of the particles to specific body compartments or cells or receptors is intended. Using small particles, e.g. having a size of less than 50 nm and preferably less than 20 nm, may enable more efficient targeting of the particles. In some embodiments, aggregates of small particles may be formed, for example, aggregates of small particles having a size of less than 50 nm and preferably less than 20 nm or even 10 nm.

The above sizes may correspond to sizes as measured by microscopic techniques (in which case the sizes represent maximum particle length) or by techniques such as dynamic light scattering or size exclusion chromatography (in which case the sizes are expressed in terms of apparent diameter, in particular, hydrodynamic diameter and stokes diameter, respectively).

### B. Immunogenic Species

The at least one immunogenic species to be delivered is associated with the respective particles formed by the amphipathic peptides and the lipids. There are several possibilities to achieve a respective association. According to one embodiment, the immunogenic species is partially or entirely lipophilic, thus allowing all or a portion of the immunogenic species to be anchored into the lipid core of the particles.

According to another embodiment, the immunogenic species is provided with a lipophilic anchor. The lipophilic anchor can be for example directly covalently attached to the immunogenic species or a linker can be used in order to allow attachment of the lipophilic anchor. The lipophilic anchor inserts into the lipid core of the particle, thereby anchoring the immunogenic species via the lipophilic anchor to the particle formed by the amphipathic peptides and the lipids.

According to another embodiment, an immunogenic species is cleaved to render it more hydrophobic or to expose a hydrophobic portion of the immunogenic species.

According to an alternative embodiment, the immunogenic species is associated with the particles by charge interactions.

According to an alternative embodiment, a hydrophilic immunogenic species may be associated with the hydrophilic face of the amphipathic peptide.

For example, when a negatively charged immunogenic species is to be delivered, a positively charged capturing agent can be used in order to capture and associate the negatively charged immunogenic species to the particle. The respective capturing agent may, for example, comprise a lipophilic anchor, which allows anchoring of the capturing agent to the particle via the lipophilic anchor which inserts into the lipid core. The capturing agent according to this embodiment would comprise cationic groups and can be for example a cationic lipid. Examples of negatively charged immunogenic species may be selected from those described elsewhere herein and include negatively charged antigens such as negatively charged peptide-containing antigens, polynucleotide-containing antigens (which expresses polypeptide-containing antigens in vivo), for instance, RNA vector constructs and DNA vector constructs (e.g., plasmid DNA) and negatively charged immunological adjuvants such as immunostimulatory oligonucleotides (e.g., CpG oligonucleotides), single-stranded RNA, etc.). The charged groups of the capturing agent are available for interaction with the negatively charged immunogenic species when the capturing agent is anchored to the particle via the lipophilic anchor. Thereby, an association of the immunogenic species with the particle is achieved.

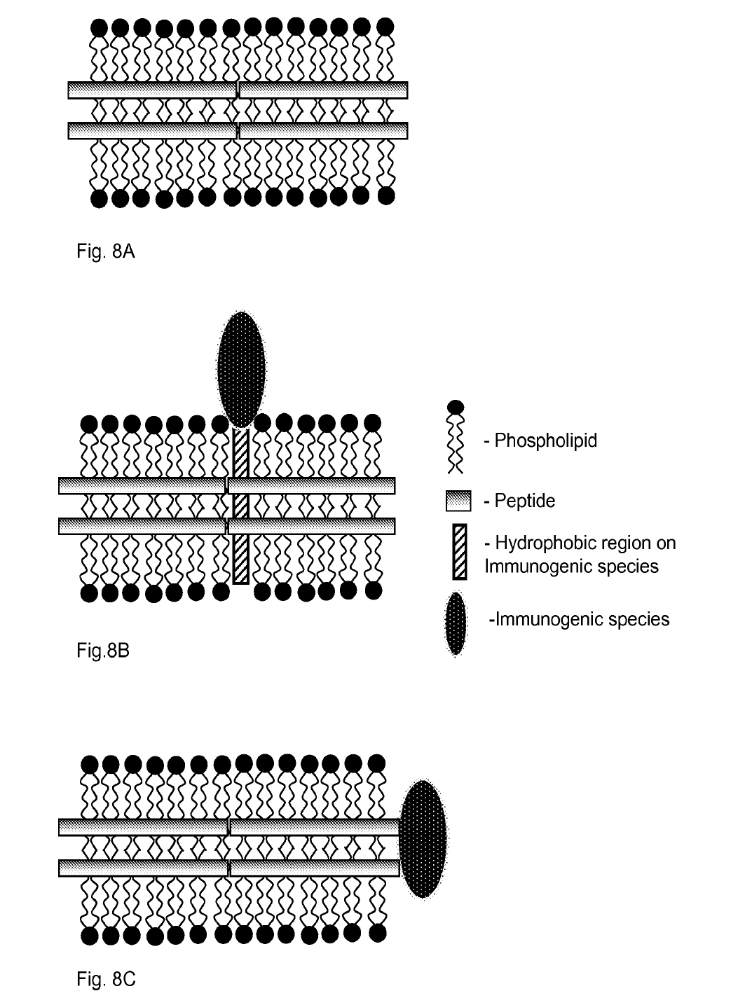

FIG. 8A schematically shows a particle comprising a phospholipid that is stabilized by an amphiphilic peptide in accordance with the invention. FIG. 8B schematically shows an immunogenic species with a hydrophobic region wherein the hydrophobic region (i.e., a lipophilic anchor) of the immunogenic species is inserted into the phospholipids, thereby anchoring the immunogenic species to the particles. FIG. 8C schematically shows a hydrophilic immunogenic species associated with the hydrophilic face of the amphipathic peptide.

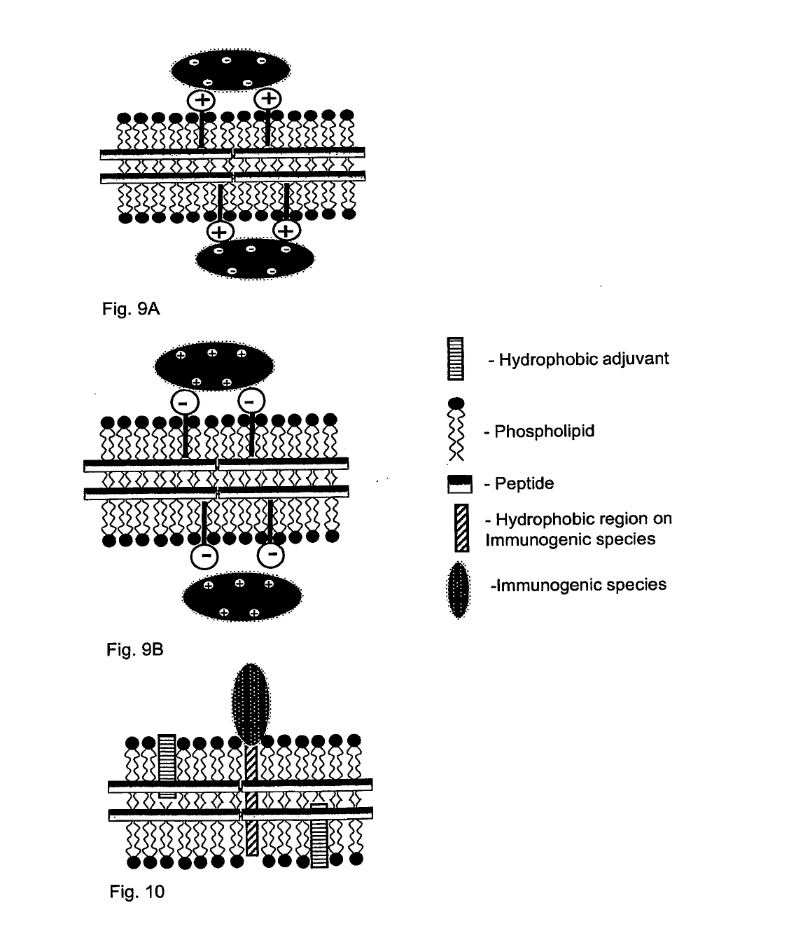

FIG. 9A schematically shows an alternative embodiment wherein cationic lipids are used as capturing agents in order to associate a negatively charged immunogenic species with the particles. The cationic lipids comprise a lipophilic anchor and a cationic head. FIG. 9B schematically shows an alternative embodiment wherein anionic lipids are used as capturing agents in order to associate a positively charged immunogenic species with the particles. The anionic lipids comprise a lipophilic anchor and an anionic head.

FIG. 10 schematically illustrates a particle comprising a phospholipid that is stabilized by an amphiphilic peptide in accordance with the invention as well as (a) an immunogenic species with a hydrophobic region wherein the hydrophobic region of the immunogenic species is inserted into the phospholipids and (b) a hydrophobic adjuvant that is inserted into the phospholipids, such that immunogenic species and adjuvant are anchored to the particles.

Also combinations of different association principles described are within the scope of the present invention. For example, a negatively charged capturing agent can be used in order to capture and associate a positively charged immunogenic species to the particle.

As seen from the above, immunogenic species for use in the present invention can be of any nature (e.g. hydrophobic, hydrophilic, partially hydrophobic and partially hydrophilic, charged, etc.) and can be for example selected from those species described elsewhere herein, among others.

As used herein, an “immunogenic species” is a chemical species that is capable of eliciting or modifying an immunological response Immunogenic species for use in the present invention include antigens and immunological adjuvants.

The term “adjuvant” refers to any substance that assists or modifies the action of a pharmaceutical, including but not limited to immunological adjuvants, which increase and/or diversify the immune response to an antigen. Hence, immunological adjuvants include compounds that are capable of potentiating an immune response to antigens. Immunological adjuvants can potentiate humoral and/or cellular immunity. Substances that stimulate an innate immune response are included within the definition of immunological adjuvants herein Immunological adjuvants may also be referred to herein as “immunopotentiators.”

As used herein, an “antigen” refers to a molecule containing one or more epitopes (e.g., linear, conformational or both) that elicit an immunological response. As used herein, an “epitope” is that portion of given species (e.g., an antigenic molecule or antigenic complex) that determines its immunological specificity. An epitope is within the scope of the present definition of antigen. Commonly, an epitope is a polypeptide or polysaccharide in a naturally occurring antigen. In artificial antigens, it can be a low molecular weight substance such as an arsanilic acid derivative.

The term “antigen” as used herein denotes both subunit antigens, i.e., antigens which are separate and discrete from a whole organism with which the antigen is associated in nature, as well as killed, attenuated or inactivated bacteria, viruses, parasites or other pathogens or tumor cells. Antibodies such as anti-idiotype antibodies, or fragments thereof, and synthetic peptide mimotopes, which can mimic an antigen or antigenic determinant, are also captured under the definition of antigen as used herein. Similarly, a polynucleotide that expresses an immunogenic protein, or antigenic determinant in vivo, such as in nucleic acid immunization applications, is also included in the definition of antigen herein.

An “immunological response” or “immune response” is the development in a subject of a humoral and/or a cellular immune response to the immunogenic species.

Immune responses include innate and adaptive immune responses. Innate immune responses are fast-acting responses that provide a first line of defense for the immune system. In contrast, adaptive immunity uses selection and clonal expansion of immune cells having somatically rearranged receptor genes (e.g., T- and B-cell receptors) that recognize antigens from a given pathogen or disorder (e.g., a tumor), thereby providing specificity and immunological memory. Innate immune responses, among their many effects, lead to a rapid burst of inflammatory cytokines and activation of antigen-presenting cells (APCs) such as macrophages and dendritic cells. To distinguish pathogens from self-components, the innate immune system uses a variety of relatively invariable receptors that detect signatures from pathogens, known as pathogen-associated molecular patterns, or PAMPs. The addition of microbial components to experimental vaccines is known to lead to the development of robust and durable adaptive immune responses. The mechanism behind this potentiation of the immune responses has been reported to involve pattern-recognition receptors (PRRs), which are differentially expressed on a variety of immune cells, including neutrophils, macrophages, dendritic cells, natural killer cells, B cells and some nonimmune cells such as epithelial and endothelial cells. Engagement of PRRs leads to the activation of some of these cells and their secretion of cytokines and chemokines, as well as maturation and migration of other cells. In tandem, this creates an inflammatory environment that leads to the establishment of the adaptive immune response. PRRs include nonphagocytic receptors, such as Toll-like receptors (TLRs) and nucleotide-binding oligomerization domain (NOD) proteins, and receptors that induce phagocytosis, such as scavenger receptors, mannose receptors and β-glucan receptors. Reported TLRs (along with examples of some reported ligands, which may be used as immunogenic species in various embodiments of the invention) include the following: TLR1 (bacterial lipoproteins from Mycobacteria, Neisseria), TLR2 (zymosan yeast particles, peptidoglycan, lipoproteins, lipopeptides, glycolipids, lipopolysaccharide), TLR3 (viral double-stranded RNA, poly:IC), TLR4 (bacterial lipopolysaccharides, plant product taxol), TLR5 (bacterial flagellins), TLR6 (yeast zymosan particles, lipotechoic acid, lipopeptides from mycoplasma), TLR7 (single-stranded RNA, imiquimod, resimiquimod, and other synthetic compounds such as loxoribine and bropirimine), TLR8 (single-stranded RNA, resimiquimod) and TLR9 (CpG oligonucleotides), among others. Dendritic cells are recognized as some of the most important cell types for initiating the priming of naive CD4⁺ helper T (T_{H}) cells and for inducing CD8⁺ T cell differentiation into killer cells. TLR signaling has been reported to play an important role in determining the quality of these helper T cell responses, for instance, with the nature of the TLR signal determining the specific type of T_{H} response that is observed (e.g., T_{H}1 versus T_{H}2 response). A combination of antibody (humoral) and cellular immunity are produced as part of a T_{H}1-type response, whereas a T_{H}2-type response is predominantly an antibody response. Various TLR ligands such as CpG DNA (TLR9) and imidazoquinolines (TLR7, TLR8) have been documented to stimulate cytokine production from immune cells in vitro. The imidazoquinolines are the first small, drug-like compounds shown to be TLR agonists. For further information, see, e.g., A. Pashine, N. M. Valiante and J. B. Ulmer, Nature Medicine 11, S63-S68 (2005), K. S. Rosenthal and D. H Zimmerman, Clinical and Vaccine Immunology, 13(8), 821-829 (2006), and the references cited therein.

For purposes of the present invention, a humoral immune response refers to an immune response mediated by antibody molecules, while a cellular immune response is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4⁺ and CD8⁺ T-cells.

A composition such as an immunogenic composition or a vaccine that elicits a cellular immune response may thus serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host. The ability of a particular antigen or composition to stimulate a cell-mediated immunological response may be determined by a number of assays known in the art, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al. (1993) J. Immunol. 151:4189-4199; Doe et al. (1994) Eur. J. Immunol. 24:2369-2376. Thus, an immunological response as used herein may be one which stimulates the production of CTLs and/or the production or activation of helper T-cells. The antigen of interest may also elicit an antibody-mediated immune response. Hence, an immunological response may include, for example, one or more of the following effects among others: the production of antibodies by, for example, B-cells; and/or the activation of suppressor T-cells and/or γδ T-cells directed specifically to an antigen or antigens present in the composition or vaccine of interest. These responses may serve, for example, to neutralize infectivity, and/or mediate antibody-complement, or antibody dependent cell cytotoxicity (ADCC) to provide protection to an immunized host. Such responses can be determined using standard immunoassays and neutralization assays, well known in the art.

Compositions in accordance with the present invention display “enhanced immunogenicity” for a given antigen when they possess a greater capacity to elicit an immune response than the immune response elicited by an equivalent amount of the antigen in a differing composition (e.g., wherein the antigen is administered as a soluble protein). Thus, a composition may display enhanced immunogenicity, for example, because the composition generates a stronger immune response, or because a lower dose or fewer doses of antigen is necessary to achieve an immune response in the subject to which it is administered. Such enhanced immunogenicity can be determined, for example, by administering a composition of the invention and an antigen control to animals and comparing assay results of the two.

### 1. Immunological Adjuvants

As noted above, one or more immunological adjuvants may be provided in the compositions of the invention Immunological adjuvants may be anchored to the lipid cores of the particle(s) formed by the amphipathic peptides and the lipids (e.g., by virtue of a lipophilic anchor that is covalently or non-covalently attached to the immunological adjuvant) or they may be otherwise combined with the particle(s) (e.g., admixed with particles to which an antigen has been anchored, etc.).

Immunological adjuvants for use with the invention include, but are not limited to, one or more of the following:

### A. Mineral Containing Compositions

Mineral containing compositions suitable for use as adjuvants include mineral salts, such as aluminum salts and calcium salts. The invention includes mineral salts such as hydroxides (e.g. oxyhydroxides), phosphates (e.g. hydroxyphosphates, orthophosphates), sulfates, etc. (see, e.g., Vaccine Design: The Subunit and Adjuvant Approach (Powell, M. F. and Newman, M. J. eds.) (New York: Plenum Press) 1995, Chapters 8 and 9), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

In one embodiment, the aluminum based adjuvant for use in the present invention is alum (aluminum potassium sulfate (AlK(SO₄)₂)), or an alum derivative, such as that formed in-situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbant, having a surface area of approximately 500 m²/g. In another embodiment, the aluminum based adjuvant is aluminum phosphate adjuvant (AlPO₄) or aluminum hydroxyphosphate, which contains phosphate groups in place of some or all of the hydroxyl groups of aluminum hydroxide adjuvant. Preferred aluminum phosphate adjuvants provided herein are amorphous and soluble in acidic, basic and neutral media.

In another embodiment, the adjuvant comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. In another embodiment, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

### B. Oil-Emulsions

Oil-emulsion compositions and formulations suitable for use as adjuvants (with or without other specific immunostimulating agents such as muramyl peptides or bacterial cell wall components) include squalene-water emulsions, such as MF59 (5% Squalene, 0.5% Tween 80, and 0.5% Span 85, formulated into submicron particles using a microfluidizer). See WO 90/14837. See also, Podda (2001) Vaccine 19: 2673-2680; Frey et al. (2003) Vaccine 21:4234-4237. MF59 is used as the adjuvant in the FLUAD™ influenza virus trivalent subunit vaccine.

Particularly preferred oil-emulsion adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyethylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (WO 90/14837; U.S. Pat. No. 6,299,884; U.S. Pat. No. 6,451,325; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) (New York: Plenum Press) 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably O-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in WO 90/14837; U.S. Pat. No. 6,299,884; and U.S. Pat. No. 6,451,325.

Complete Freund's adjuvant (CFA) and incomplete Freund's adjuvant (IFA) may also be used as adjuvants in the invention.

### C. Saponin Formulations

Saponin formulations are also suitable for use as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. Saponin adjuvant formulations include STIMULON® adjuvant (Antigenics, Inc., Lexington, Mass.).

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of (an) additional detergent(s). See WO 00/07621.

A review of the development of saponin based adjuvants can be found in Barr et al. (1998) Adv. Drug Del. Rev. 32:247-271. See also Sjolander et al. (1998) Adv. Drug Del. Rev. 32:321-338.

### D. Virosomes and Virus Like Particles (VLPs)

Virosomes and Virus Like Particles (VLPs) are also suitable as adjuvants. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480; WO 03/024481; Niikura et al. (2002) Virology 293:273-280; Lenz et al. (2001) J. Immunol. 166(9):5346-5355; Pinto et al. (2003) J. Infect. Dis. 188:327-338; and Gerber et al. (2001) J. Virol. 75(10):4752-4760. Virosomes are discussed further in, for example, Gluck et al. (2002) Vaccine 20:B10-B16. Immunopotentiating reconstituted influenza virosomes (IRIV) are used as the subunit antigen delivery system in the intranasal trivalent INFLEXAL™ product (Mischler and Metcalfe (2002) Vaccine 20 Suppl 5:B17-B23) and the INFLUVAC PLUS™ product.

### E. Bacterial or Microbial Derivatives

Adjuvants suitable for use in the invention include bacterial or microbial derivatives such as:

(1) Non-toxic derivatives of enterobacterial lipopolysaccharide (LPS): Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives, e.g., RC-529. See Johnson et al. (1999) Bioorg. Med. Chem. Lett. 9:2273-2278.

(2) Lipid A Derivatives: Lipid A derivatives include derivatives of lipid A from Escherichia coli such as OM-174. OM-174 is described for example in Meraldi et al. (2003) Vaccine 21:2485-2491; and Pajak et al. (2003) Vaccine 21:836-842.

Another exemplary adjuvant is the synthetic phospholipid dimer, E6020 (Eisai Co. Ltd., Tokyo, Japan), which mimics the physicochemical and biological properties of many of the natural lipid A's derived from Gram-negative bacteria.

(3) Immunostimulatory oligonucleotides: Immunostimulatory oligonucleotides or polymeric molecules suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory. The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla et al. (2003) Nucl. Acids Res. 31(9): 2393-2400; WO 02/26757; and WO 99/62923 for examples of possible analog substitutions. The adjuvant effect of CpG oligonucleotides is further discussed in Krieg (2003) Nat. Med. 9(7):831-835; McCluskie et al. (2002) FEMS Immunol. Med. Microbiol. 32:179-185; WO 98/40100; U.S. Pat. No. 6,207,646; U.S. Pat. No. 6,239,116; and U.S. Pat. No. 6,429,199.

The CpG sequence may be directed to TLR9, such as the motif GTCGTT or TTCGTT. See Kandimalla et al. (2003) Biochem. Soc. Trans. 31 (part 3):654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell et al. (2003) J. Immunol. 170(8):4061-4068; Krieg (2002) TRENDS Immunol. 23(2): 64-65; and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla et al. (2003) BBRC 306:948-953; Kandimalla et al. (2003) Biochem. Soc. Trans. 31 (part 3):664-658′ Bhagat et al. (2003) BBRC 300:853-861; and WO03/035836.

Immunostimulatory oligonucleotides and polymeric molecules also include alternative polymer backbone structures such as, but not limited to, polyvinyl backbones (Pitha et al. (1970) Biochem. Biophys. Acta 204(1):39-48; Pitha et al. (1970) Biopolymers 9(8):965-977), and morpholino backbones (U.S. Pat. No. 5,142,047; U.S. Pat. No. 5,185,444). A variety of other charged and uncharged polynucleotide analogs are known in the art. Numerous backbone modifications are known in the art, including, but not limited to, uncharged linkages (e.g., methyl phosphonates, phosphotriesters, phosphoamidates, and carbamates) and charged linkages (e.g., phosphorothioates and phosphorodithioates).

Adjuvant IC31, Intercell AG, Vienna, Austria, is a synthetic formulation that contains an antimicrobial peptide, KLK, and an immunostimulatory oligonucleotide, ODN1a. The two component solution may be simply mixed with antigens (e.g., particles in accordance with the invention with an associated antigen), with no conjugation required.

(4) ADP-ribosylating toxins and detoxified derivatives thereof: Bacterial ADP-ribosylating toxins and detoxified derivatives thereof may be used as adjuvants in the invention. Preferably, the protein is derived from E. coli (i.e., E. coli heat labile enterotoxin “LT”), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon et al. (2002) Infect. Immun. 70(6):3012-3019; Pizza et al. (2001) Vaccine 19:2534-2541; Pizza et al. (2000) Int. J. Med. Microbiol. 290(4-5):455-461; Scharton-Kersten et al. (2000) Infect. Immun. 68(9):5306-5313; Ryan et al. (1999) Infect. Immun. 67(12):6270-6280; Partidos et al. (1999) Immunol. Lett. 67(3):209-216; Peppoloni et al. (2003) Vaccines 2(2):285-293; and Pine et al. (2002) J. Control Release 85(1-3):263-270. Numerical reference for amino acid substitutions is preferably based on the alignments of the A and B subunits of ADP-ribosylating toxins set forth in Domenighini et al. (1995) Mol. Microbiol. 15(6):1165-1167.

### F. Bioadhesives and Mucoadhesives

Bioadhesives and mucoadhesives may also be used as adjuvants. Suitable bioadhesives include esterified hyaluronic acid microspheres (Singh et al. (2001) J. Cont. Release 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention (see WO 99/27960).

### G. Liposomes

Examples of liposome formulations suitable for use as adjuvants are described in U.S. Pat. No. 6,090,406; U.S. Pat. No. 5,916,588; and EP Patent Publication No. EP 0 626 169.

### H. Polyoxyethylene Ether and Polyoxyethylene Ester Formulations

Adjuvants suitable for use in the invention include polyoxyethylene ethers and polyoxyethylene esters (see, e.g., WO 99/52549). Such formulations further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### I. Polyphosphazene (PCPP)

PCPP formulations suitable for use as adjuvants are described, for example, in Andrianov et al. (1998) Biomaterials 19(1-3):109-115; and Payne et al. (1998) Adv. Drug Del. Rev. 31(3):185-196.

### J. Muramyl Peptides

Examples of muramyl peptides suitable for use as adjuvants include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### K. Imidazoquinoline Compounds

Examples of imidazoquinoline compounds suitable for use as adjuvants include Imiquimod and its analogues, which are described further in Stanley (2002) Clin. Exp. Dermatol. 27(7):571-577; Jones (2003) Curr. Opin. Investig. Drugs 4(2):214-218; and U.S. Pat. Nos. 4,689,338; 5,389,640; 5,268,376; 4,929,624; 5,266,575; 5,352,784; 5,494,916; 5,482,936; 5,346,905; 5,395,937; 5,238,944; and 5,525,612.

Preferred imidazoquinolines for the practice of the present invention include imiquimod, resiquimod, and

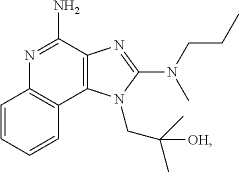

the latter of which is also referred to herein as “imidazoquinoline 090”. See, e.g., Int. Pub. Nos. WO 2006/031878 to Valiante et al. and WO 2007/109810 to Sutton et al. Such compounds are known to be TLR7 agonists.
l. Thiosemicarbazone Compounds

Examples of thiosemicarbazone compounds suitable for use as adjuvants, as well as methods of formulating, manufacturing, and screening for such compounds, include those described in WO 04/60308. The thiosemicarbazones are particularly effective in the stimulation of human peripheral blood mononuclear cells for the production of cytokines, such as TNF-α.

m. Tryptanthrin Compounds

Examples of tryptanthrin compounds suitable for use as adjuvants, as well as methods of formulating, manufacturing, and screening for such compounds, include those described in WO 04/64759. The tryptanthrin compounds are particularly effective in the stimulation of human peripheral blood mononuclear cells for the production of cytokines, such as TNF-α.

n. Human Immunomodulators

Human immunomodulators suitable for use as adjuvants include cytokines, such as interleukins (e.g. IL-1, IL-2, IL-4, IL-5, IL-6, IL-7, IL-12, etc.), interferons (e.g. interferon-γ), macrophage colony stimulating factor (M-CSF), and tumor necrosis factor (TNF).

o. Lipopeptides

Lipopeptides (i.e., compounds comprising one or more fatty acid residues and two or more amino acid residues) are also known to have immunostimulating character. Lipopeptides based on glycerylcysteine are of particularly suitable for use as adjuvants. Specific examples of such peptides include compounds of the following formula

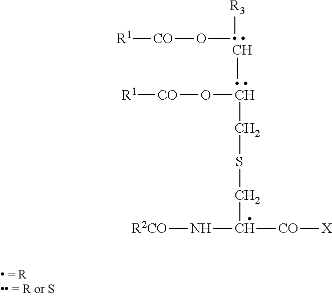

in which each of R¹ and R² represents a saturated or unsaturated, aliphatic or mixed aliphatic-cycloaliphatic hydrocarbon radical having from 8 to 30, preferably 11 to 21, carbon atoms that is optionally also substituted by oxygen functions, R³ represents hydrogen or the radical R₁—CO—O—CH₂— in which R¹ has the same meaning as above, and X represents an amino acid bonded by a peptide linkage and having a free, esterified or amidated carboxy group, or an amino acid sequence of from 2 to 10 amino acids of which the terminal carboxy group is in free, esterified or amidated form. In certain embodiments, the amino acid sequence comprises a D-amino acid, for example, D-glutamic acid (D-Glu) or D-gamma-carboxy-glutamic acid (D-Gla).

Bacterial lipopeptides generally recognize TLR2, without requiring TLR6 to participate. (TLRs operate cooperatively to provide specific recognition of various triggers, and TLR2 plus TLR6 together recognize peptidoglycans, while TLR2 recognizes lipopeptides without TLR6.) These are sometimes classified as natural lipopeptides and synthetic lipopeptides. Synthetic lipopeptides tend to behave similarly, and are primarily recognized by TLR2.

Lipopeptides suitable for use as adjuvants include compounds of Formula I:

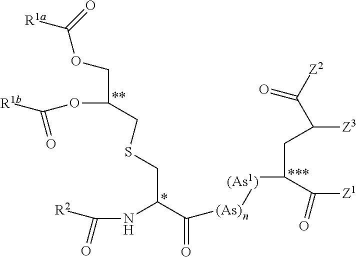

where the chiral center labeled * and the one labeled *** are both in the R configuration;

the chiral center labeled ** is either in the R or S configuration;

each R^{1a} and R^{1b} is independently an aliphatic or cycloaliphatic-aliphatic hydrocarbon group having 7-21 carbon atoms, optionally substituted by oxygen functions, or one of R^{1a} and R^{1b}, but not both, is H;

R² is an aliphatic or cycloaliphatic hydrocarbon group having 1-21 carbon atoms and optionally substituted by oxygen functions;

n is 0 or 1;

As represents either —O-Kw-CO— or —NH-Kw-CO—, where Kw is an aliphatic hydrocarbon group having 1-12 carbon atoms;

As¹ is a D- or L-alpha-amino acid;

Z¹ and Z² each independently represent —OH or the N-terminal radical of a D- or L-alpha amino acid of an amino-(lower alkane)-sulfonic acid or of a peptide having up to 6 amino acids selected from the D- and L-alpha aminocarboxylic acids and amino-lower alkyl-sulfonic acids; and

Z³ is H or —CO—Z⁴, where Z⁴ is —OH or the N-terminal radical of a D- or L-alpha amino acid of an amino-(lower alkane)-sulfonic acid or of a peptide having up to 6 amino acids selected from the D and L-alpha aminocarboxylic acids and amino-lower alkyl-sulfonic acids;

or an ester or amide formed from the carboxylic acid of such compounds. Suitable amides include —NH₂ and NH(lower alkyl), and suitable esters include C1-C4 alkyl esters. (lower alkyl or lower alkane, as used herein, refers to C₁-C₆ straight chain or branched alkyls).

Such compounds are described in more detail in U.S. Pat. No. 4,666,886. In one preferred embodiment, the lipopeptide is of the following formula:

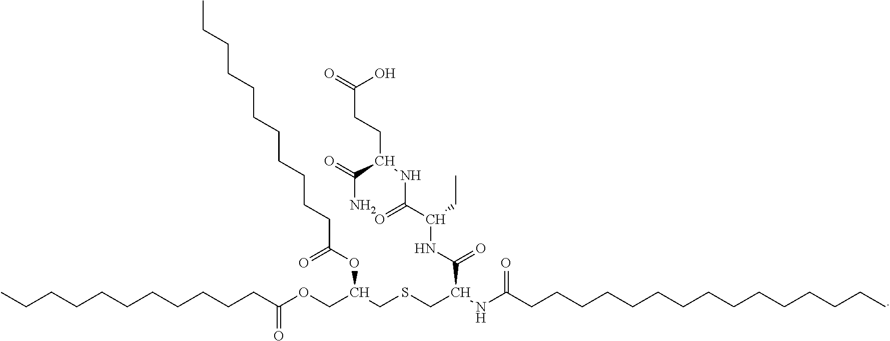

Another example of a lipopeptide species is called LP40, and is an agonist of TLR2. Akdis, et al., Eur. J. Immunology, 33: 2717-26 (2003).

These are related to a known class of lipopeptides from E. coli, referred to as murein lipoproteins. Certain partial degradation products of those proteins called murein lipopetides are described in Hantke, et al., Eur. J. Biochem., 34: 284-296 (1973). These comprise a peptide linked to N-acetyl muramic acid and are thus related to Muramyl peptides, which are described in Baschang, et al., Tetrahedron, 45(20): 6331-6360 (1989).

p. Benzonaphthyridines

Examples of benzonaphthyridine compounds suitable for use as adjuvants include compounds having the structure of Formula (I), and pharmaceutically acceptable salts, solvates, N-oxides, prodrugs and isomers thereof:

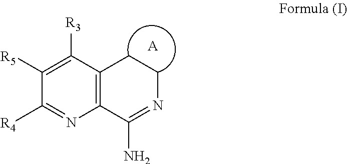

wherein:
R³ is H, halogen, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₁-C₆alkoxy, C₁-C₆haloalkoxy, C₃-C₈cycloalkyl, or C₃-C₈heterocycloalkyl groups of R³ are each optionally substituted with 1 to 3 substituents independently selected from halogen, —CN, —R⁷, —OR⁸, —C(O)R⁸, —OC(O)R⁸, —C(O)OR⁸, —N(R⁹)₂, —C(O)N(R⁹)₂, —S(O)₂R⁸, —S(O)₂N(R⁹)₂ and —NR⁹S(O)₂R⁸;
R⁴ and R⁵ are each independently selected from H, halogen, —C(O)OR⁷, —C(O)R⁷, —C(O)N(R¹¹R¹²), —N(R¹¹R¹²), —N(R⁹)₂, —NHN(R⁹)₂, —SR⁷, —(CH₂)ₙOR⁷, —(CH₂)ₙR⁷, -LR^{B}, -LR¹⁰, —OLR⁸, —OLR¹⁰, C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂, —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
or R³ and R⁴, or R⁴ and R⁵, when present on adjacent ring atoms, can optionally be linked together to form a 5-6 membered ring, wherein the 5-6 membered ring is optionally substituted with R⁷;
each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl groups of R⁷ are each optionally substituted with 1 to 3 R¹³ groups;
each R⁸ is independently selected from H, —CH(R¹⁰)₂, C₁-C₈alkyl, C₂-C₈alkene, C₂-C₈alkyne, C₁-C₆haloalkyl, C₁-C₆alkoxy, C₁-C₆heteroalkyl, C₃-C₈cycloalkyl, C₂-C₈heterocycloalkyl, C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆haloalkyl, C₁-C₆alkoxy, C₃-C₈cycloalkyl, C₂-C₈heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹⁰, —NR¹¹R¹², —N(R⁹)₂, —OR⁹, —OR¹⁰, —C(O)NR¹¹R¹², —C(O)NR¹¹OH, —S(O)₂R¹¹, —S(O)₂NR¹¹R¹², —NR¹¹S(O)₂R¹¹, —P(O)(OR¹¹)₂, and —OP(O)(OR¹¹)₂;
each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆ cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, —OC(O)R¹¹, —C(O)OR¹¹, —NR¹¹R¹², —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂, and —OP(O)(OR¹¹)₂;
each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen, —R⁸, —OR⁸, -LR⁹, -LOR⁹, —N(R⁹)₂, —NR⁹C(O)R⁸, —NR⁹CO₂R⁸, —CO₂R⁸, —C(O)R⁸ and —C(O)N(R⁹)₂;
R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR⁸, —C(O)R⁸, —OC(O)R⁸, —C(O)OR⁸, —N(R⁹)₂, —NR⁸C(O)R⁸, —NR⁸C(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
each R¹³ is independently selected from halogen, —CN, -LR⁹, -LOR⁹, —OLR⁹, —LR¹⁰, -LOR¹⁰, —OLR¹⁰, -LR^{B}, -LOR⁸, —OLR⁸, -LSR⁸, -LSR¹⁰, -LC(O)R⁸, —OLC(O)R⁸, -LC(O)OR⁸, -LC(O)R¹⁰, -LOC(O)OR⁸, -LC(O)NR⁹R¹¹, -LC(O)NR⁹R⁸, -LN(R⁹)₂, -LNR⁹R⁸, -LNR⁹R¹⁰, -L=NOH, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, —OLS(O)₂N(R⁹)₂, -LNR⁹S(O)₂R⁸, -LC(O)NR⁹LN(R⁹)₂, -LP(O)(OR⁸)₂, LOP(O)(OR⁸)₂, -LP(O)(OR¹⁰)₂ and —OLP(O)(OR¹⁰)₂;
Ring A is an aryl or a heteroaryl, wherein the aryl and heteroaryl groups of Ring A are optionally substituted with 1 to 3 R^{A} groups, wherein each R^{A} is independently selected from halogen, —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —CO₂R⁸, —(CH₂)ₙCO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₂R⁸, —C(═NH)—N(R⁹)₂, and —(CH₂)₆NHC(O)R⁸; or two adjacent R^{A} substituents on Ring A form a 5-6 membered ring that contains up to two heteroatoms as ring members;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8.

In certain embodiments of compounds of Formulas (I), ring A an aromatic ring, such as phenyl, pyridyl, or pyrimidinyl, which can be substituted with the same substituents with optionally substituted C₁-C₄ alkyl or C₁-C₄ alkoxy, and each of R³, R⁴, and R⁵ independently represent H, halo, or an optionally substituted C₁-C₄ alkyl or optionally substituted C₁-C₄ alkoxy group. In certain embodiments, R³ and R⁵ each represent H.

In these compounds, R⁴ is typically an optionally substituted C₁-C₄ alkyl, and in some embodiments, R⁴ is C₁-C₄ alkyl substituted with an optionally substituted phenyl ring or heteroaryl ring (e.g., pyridine, pyrimidine, indole, thiophene, furan, oxazole, isoxazole, benzoxazole, benzimidazole, and the like). In some of these embodiments, R⁵ is H. The optionally substituted phenyl or hereoaryl ring can have up to three substituents selected from Me, CN, CF₃, halo, OMe, NH₂, NHMe, NMe₂, and optionally substituted C₁-C₄ alkyl or C₁-C₄ alkoxy, wherein substituents for the optionally substituted C₁-C₄ alkyl or C₁-C₄ alkoxy groups in Formula (I) are selected from halo, —OH, —OMe, C₁-C₄ alkyl, C₁-C₄ alkoxy, COOH, —PO₃H₂, —OPO₃H₂, NH₂, NMe₂, C₃-C₆ cycloalkyl, aryl (preferably phenyl or substituted phenyl), C₅-C₆ heterocyclyl (e.g, piperidine, morpholine, thiomorpholine, pyrrolidine); and the pharmaceutically acceptable salts of these compounds.

Other examples of benzonaphthyridine compounds suitable for use as adjuvants include compounds of Formula (II):

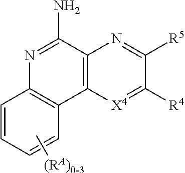

where each R^{A} is independently halo, CN, NH₂, NHMe, NMe₂, or optionally substituted C₁-C₄ alkyl or optionally substituted C₁-C₄ alkoxy; X⁴ is CH or N;

and R⁴ and R⁵ independently represent H or an optionally substituted alkyl or optionally substituted alkoxy group.

Preferably compounds of Formula (II) have 0-1 R^{A} substituents present.

In these compounds, R⁴ is typically an optionally substituted C₁-C₄ alkyl, and in some embodiments, R⁴ is C₁-C₄ alkyl substituted with an optionally substituted phenyl ring or heteroaryl ring (e.g., pyridine, pyrimidine, indole, thiophene, furan, oxazole, isoxazole, benzoxazole, benzimidazole, and the like). In some of these embodiments, R⁵ is H. The optionally substituted phenyl or hereoaryl ring can have up to three substituents selected from Me, CN, CF₃, halo, OMe, NH₂, NHMe, NMe₂, and optionally substituted C₁-C₄ alkyl or C₁-C₄ alkoxy, wherein substituents for the optionally substituted C₁-C₄ alkyl or C₁-C₄ alkoxy groups in Formula (X) are selected from halo, —OH, —OMe, C₁-C₄ alkyl, C₁-C₄ alkoxy, COOH, —PO₃H₂, —OPO₃H₂, NH₂, NMe₂, C₃-C₆ cycloalkyl, aryl (preferably phenyl or substituted phenyl), C₅-C₆ heterocyclyl (e.g, piperidine, morpholine, thiomorpholine, pyrrolidine); and the pharmaceutically acceptable salts of these compounds.

Additional examples of benzonaphthyridine compounds suitable for use as adjuvants include:

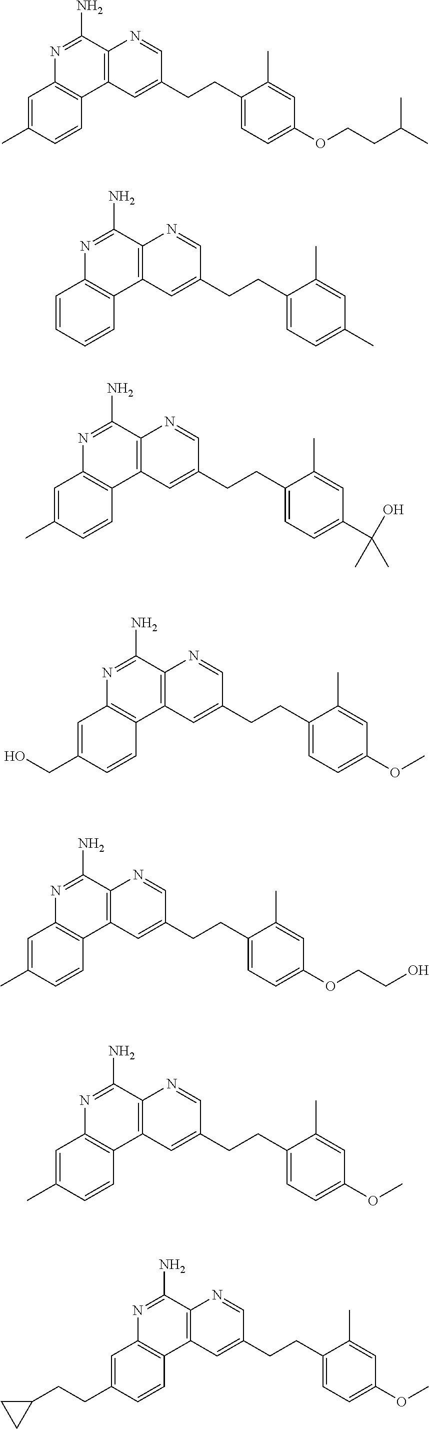

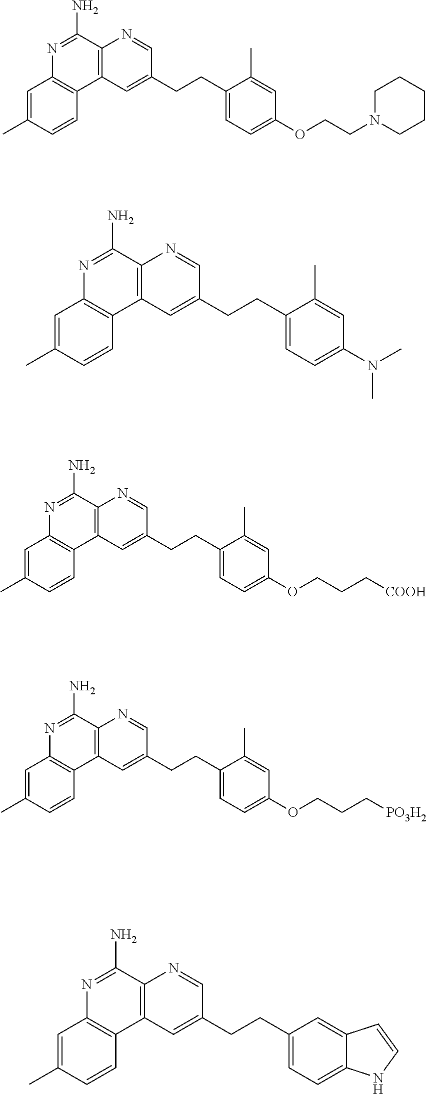

Other examples of benzonaphthyridine compounds suitable for use as adjuvants, as well as methods of formulating and manufacturing, include those described in International Application No. PCT/US2009/35563, which is incorporated herein by reference in its entirety.

The invention may also comprise combinations of aspects of one or more of the adjuvants identified above. For example, the following adjuvant compositions may be used in the invention:

(1) a saponin and an oil-in-water emulsion (WO 99/11241);
(2) a saponin (e.g., QS21)+a non-toxic LPS derivative (e.g. 3dMPL) (see WO 94/00153);
(3) a saponin (e.g., QS21)+a non-toxic LPS derivative (e.g. 3dMPL)+a cholesterol;
(4) a saponin (e.g., QS21)+3dMPL+IL-12 (optionally+a sterol) (WO 98/57659);
(5) combinations of 3dMPL with, for example, QS21 and/or oil-in-water emulsions (see EP 0 835 318; EP 0 735 898; and EP 0 761 231);
(6) SAF, containing 10% Squalane, 0.4% Tween 80, 5% pluronic-block polymer L121, and thr-MDP, either microfluidized into a submicron emulsion or vortexed to generate a larger particle size emulsion;
(7) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);
(8) one or more mineral salts (such as an aluminum salt)+a non-toxic derivative of LPS (such as 3dPML);
(9) one or more mineral salts (such as an aluminum salt)+an immunostimulatory oligonucleotide (such as a nucleotide sequence including a CpG motif).

2. Antigens

As noted above, one or more antigens may be provided in the immunogenic compositions provided herein. Antigens may be anchored to the lipid cores of the particle(s) formed by the amphipathic peptides and the lipids (e.g., by virtue of a lipophilic anchor that is covalently or non-covalently attached to the antigen) or they may be otherwise combined with the particle(s) (e.g., admixed with particles to which an immunological adjuvant has been anchored, etc.). Lipophilic peptide anchors will commonly be rich in hydrophobic amino acid residues such as residues of glycine, alanine, valine, leucine, isoleucine, methionine, phenylalanine, tryptophan and proline, among others.

In certain embodiments, antigens are selected that are at least partially lipophilic in native form (e.g., transmembrane proteins). Examples of such antigens include those that have membrane anchoring regions still present, or those that can be expressed with such regions in place.

Specific examples include influenza hemagglutinin (HA), Respiratory Syncytial Virus Antigen (RSV) F and G protein antigens, HIV envelope glycoprotein, Coronavirus S, Parainfluenza virus F, Measles F, Mumps F, Measles H, Human metapneumovirus F, Parainfluenza virus HN, Influenza NA, Hepatitis C virus E1 and E2, Flavivirus (including dengue virus, West Nile virus, Japanese encephalitis virus, yellow fever virus, tick borne encephalitis virus, etc.) M, prM, and E, Rabies virus G, Filovirus (Ebola and Marburg viruses) GP, herpes simplex virus gB and gD, and human cytomegalovirus gB, gH, gL and gO, among many others.

In some embodiments, an antigen is cleaved to render the antigen more hydrophobic or to ensure that hydrophobic portions of the protein are exposed. Such a species can cleaved in the presence of the particle formed by the amphipathic peptides and the lipids of the invention. For instance, in Example 13 below, an RSV F antigen mutation is created which is susceptible to trypsin cleavage, which results in a truncated antigen with the fusion peptide exposed. Without wishing to be bound by theory, it is believed that the exposure of the hydrophobic fusion peptide results in enhanced anchorage to the lipid cores of the particle(s) of the invention.

In some embodiments, a protein is synthesized in the presence of the particles formed by the amphipathic peptides and the lipids. For example, in Examples 15 and 16 below, M2e-TM (a model influenza protein) and bacteriorhodopisin are formed in the presence of the particles using a cell free protein expression kit. This may be advantageous, for example, where a hydrophobic or amphiphilic immunogenic protein is employed which may otherwise form insoluble aggregates. Without wishing to be bound by theory, it is believed that the protein is taken up by the particles as it is translated and expressed, thereby preventing the formation of insoluble aggregates.

Antigens for use with the immunogenic compostions herein further include, but are not limited to, one or more of the following antigens set forth below, or antigens derived from one or more of the pathogens set forth below.

### Bacterial Antigens

Bacterial antigens suitable for use with the immunogenic compositions herein include, but are not limited to, proteins, polysaccharides, lipopolysaccharides, and outer membrane vesicles which are isolated, purified or derived from a bacteria. In certain embodiments, the bacterial antigens include bacterial lysates and inactivated bacteria formulations. In certain embodiments, the bacterial antigens are produced by recombinant expression. In certain embodiments, the bacterial antigens include epitopes which are exposed on the surface of the bacteria during at least one stage of its life cycle. Bacterial antigens are preferably conserved across multiple serotypes. In certain embodiments, the bacterial antigens include antigens derived from one or more of the bacteria set forth below as well as the specific antigens examples identified below:
Neisseria meningitidis: Meningitidis antigens include, but are not limited to, proteins, saccharides (including a polysaccharide, oligosaccharide, lipooligosaccharide or lipopolysaccharide), or outer-membrane vesicles purified or derived from N. meningitides serogroup such as A, C, W135, Y, X and/or B. In certain embodiments meningitides protein antigens are be selected from adhesions, autotransporters, toxins, Fe acquisition proteins, and membrane associated proteins (preferably integral outer membrane protein).
Streptococcus pneumoniae: Streptococcus pneumoniae antigens include, but are not limited to, a saccharide (including a polysaccharide or an oligosaccharide) and/or protein from Streptococcus pneumoniae. The saccharide may be a polysaccharide having the size that arises during purification of the saccharide from bacteria, or it may be an oligosaccharide achieved by fragmentation of such a polysaccharide. In the 7-valent PREVNAR™ product, for instance, 6 of the saccharides are presented as intact polysaccharides while one (the 18C serotype) is presented as an oligosaccharide. In certain embodiments saccharide antigens are selected from one or more of the following pneumococcal serotypes 1, 2, 3, 4, 5, 6A, 6B, 7F, 8, 9N, 9V, 10A, 11A, 12F, 14, 15B, 17F, 18C, 19A, 19F, 20, 22F, 23F, and/or 33F. An immunogenic composition may include multiple serotypes e.g. 2, 3, 4, 5, 6, 7, 8, 9, 10, 11, 12, 13, 14, 15, 16, 17, 18, 19, 20, 21, 22, 23 or more serotypes. 7-valent, 9-valent, 10-valent, 11-valent and 13-valent conjugate combinations are already known in the art, as is a 23-valent unconjugated combination. For example, an 10-valent combination may include saccharide from serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F. etc. In certain embodiments, protein antigens may be selected from a protein identified in WO98/18931, WO98/18930, U.S. Pat. No. 6,699,703, U.S. Pat. No. 6,800,744, WO97/43303, WO97/37026, WO 02/079241, WO 02/34773, WO 00/06737, WO 00/06738, WO 00/58475, WO 2003/082183, WO 00/37105, WO 02/22167, WO 02/22168, WO 2003/104272, WO 02/08426, WO 01/12219, WO 99/53940, WO 01/81380, WO 2004/092209, WO 00/76540, WO 2007/116322, LeMieux et al., Infect. Imm. (2006) 74:2453-2456, Hoskins et al., J. Bacteriol. (2001) 183:5709-5717, Adamou et al., Infect. Immun (2001) 69(2):949-958, Briles et al., J. Infect. Dis. (2000) 182:1694-1701, Talkington et al., Microb. Pathog. (1996) 21(1):17-22, Bethe et al., FEMS Microbiol. Lett. (2001) 205(1):99-104, Brown et al., Infect. Immun. (2001) 69:6702-6706, Whalen et al., FEMS Immunol. Med. Microbiol. (2005) 43:73-80, Jomaa et al., Vaccine (2006) 24(24):5133-5139. In other embodiments, Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, SpIO1, Sp130, Sp125, Sp133, pneumococcal pilus subunits.
Streptococcus pyogenes (Group A Streptococcus): Group A Streptococcus antigens include, but are not limited to, a protein identified in WO 02/34771 or WO 2005/032582 (including GAS 40), fusions of fragments of GAS M proteins (including those described in WO 02/094851, and Dale, Vaccine (1999) 17:193-200, and Dale, Vaccine 14(10): 944-948), fibronectin binding protein (Sfb1), Streptococcal heme-associated protein (Shp), and Streptolysin S (SagA).
Moraxella catarrhalis: Moraxella antigens include, but are not limited to, antigens identified in WO 02/18595 and WO 99/58562, outer membrane protein antigens (HMW-OMP), C-antigen, and/or LPS.
Bordetella pertussis: Pertussis antigens include, but are not limited to, pertussis holotoxin (PT) and filamentous haemagglutinin (FHA) from B. pertussis, optionally also combination with pertactin and/or agglutinogens 2 and 3.
Burkholderia: Burkholderia antigens include, but are not limited to Burkholderia mallei, Burkholderia pseudomallei and Burkholderia cepacia.
Staphylococcus aureus: Staph aureus antigens include, but are not limited to, a polysaccharide and/or protein from S. aureus. S. aureus polysaccharides include, but are not limited to, type 5 and type 8 capsular polysaccharides (CP5 and CP8) optionally conjugated to nontoxic recombinant Pseudomonas aeruginosa exotoxin A, such as StaphVAX™, type 336 polysaccharides (336PS), polysaccharide intercellular adhesions (PIA, also known as PNAG). S. aureus proteins include, but are not limited to, antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin). In certain embodiments, S. aureus antigens may be selected from a protein identified in WO 02/094868, WO 2008/019162, WO 02/059148, WO 02/102829, WO 03/011899, WO 2005/079315, WO 02/077183, WO 99/27109, WO 01/70955, WO 00/12689, WO 00/12131, WO 2006/032475, WO 2006/032472, WO 2006/032500, WO 2007/113222, WO 2007/113223, WO 2007/113224. In other embodiments, S. aureus antigens may be selected from IsdA, IsdB, IsdC, SdrC, SdrD, SdrE, ClfA, ClfB, SasF, SasD, SasH (AdsA), Spa, EsaC, EsxA, EsxB, Emp, HlaH35L, CPS, CP8, PNAG, 336PS.
Staphylococcus epidermis: S. epidermidis antigens include, but are not limited to, slime-associated antigen (SAA).
Clostridium tetani (Tetanus): Tetanus antigens include, but are not limited to, tetanus toxoid (TT). In certain embodiments such antigens are used as a carrier protein in conjunction/conjugated with the immunogenic compositions provided herein.
Clostridium perfringens: Antigens include, but are not limited to, Epsilon toxin from Clostridium perfringen.
Clostridium botulinums (Botulism): Botulism antigens include, but are not limited to, those derived from C. botulinum.
Cornynebacterium diphtheriae (Diphtheria): Diphtheria antigens include, but are not limited to, diphtheria toxin, preferably detoxified, such as CRM₁₉₇. Additionally antigens capable of modulating, inhibiting or associated with ADP ribosylation are contemplated for combination/co-administration/conjugation with the immunogenic compositions provided herein. In certain embodiments, the diphtheria toxoids are used as carrier proteins.
Haemophilus influenzae B (Hib): Hib antigens include, but are not limited to, a Hib saccharide antigen.
Pseudomonas aeruginosa: Pseudomonas antigens include, but are not limited to, endotoxin A, Wzz protein, P. aeruginosa LPS, LPS isolated from PAO1 (O5 serotype), and/or Outer Membrane Proteins, including Outer Membrane Proteins F (OprF).
Legionella pneumophila. Bacterial antigens derived from Legionella pneumophila.
Coxiella burnetii. Bacterial antigens derived from Coxiella burnetii.
Brucella. Bacterial antigens derived from Brucella, including but not limited to, B. abortus, B. canis, B. melitensis, B. neotomae, B. ovis, B. suis and B. pinnipediae.
Francisella. Bacterial antigens derived from Francisella, including but not limited to, F. novicida, F. philomiragia and F. tularensis.
Streptococcus agalactiae (Group B Streptococcus): Group B Streptococcus antigens include, but are not limited to, a protein or saccharide antigen identified in WO 02/34771, WO 03/093306, WO 04/041157, or WO 2005/002619 (including proteins GBS 80, GBS 104, GBS 276 and GBS 322, and including saccharide antigens derived from serotypes Ia, Ib, Ia/c, II, III, IV, V, VI, VII and VIII).
Neiserria gonorrhoeae: Gonorrhoeae antigens include, but are not limited to, Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al, J Infectious Disease (2000) 182:848-855), also see, e.g., WO99/24578, WO99/36544, WO99/57280, WO02/079243).
Chlamydia trachomatis: Chlamydia trachomatis antigens include, but are not limited to, antigens derived from serotypes A, B, Ba and C (agents of trachoma, a cause of blindness), serotypes L1, L2 & L3 (associated with Lymphogranuloma venereum), and serotypes, D-K. In certain embodiments, chlamydia trachomas antigens include, but are not limited to, an antigen identified in WO 00/37494, WO 03/049762, WO 03/068811, or WO 05/002619, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).
Treponema pallidum (Syphilis): Syphilis antigens include, but are not limited to, TmpA antigen.
Haemophilus ducreyi (causing chancroid): Ducreyi antigens include, but are not limited to, outer membrane protein (DsrA).
Enterococcus faecalis or Enterococcus faecium: Antigens include, but are not limited to, a trisaccharide repeat or other Enterococcus derived antigens.
Helicobacter pylori: H pylori antigens include, but are not limited to, Cag, Vac, Nap, HopX, HopY and/or urease antigen.
Staphylococcus saprophyticus: Antigens include, but are not limited to, the 160 kDa hemagglutinin of S. saprophyticus antigen.
Yersinia enterocolitica Antigens include, but are not limited to, LPS.
E. coli: E. coli antigens may be derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC antigens include, but are not limited to, accessory colonization factor (orf3526), orf353, bacterial Ig-like domain (group 1) protein (orf405), orf1364, NodT-family outer-membrane-factor-lipoprotein efflux transporter (orf1767), gspK (orf3515), gspJ (orf3516), tonB-dependent siderophore receptor (orf3597), fimbrial protein (orf3613), upec-948, upec-1232, A chain precursor of the type-1 fimbrial protein (upec-1875), yap H homolog (upec-2820), and hemolysin A (recp-3768).
Bacillus anthracis (anthrax): B. anthracis antigens include, but are not limited to, A-components (lethal factor (LF) and edema factor (EF)), both of which can share a common B-component known as protective antigen (PA). In certain embodiments, B. anthracis antigens are optionally detoxified.
Yersinia pestis (plague): Plague antigens include, but are not limited to, F1 capsular antigen, LPS, Yersinia pestis V antigen.
Mycobacterium tuberculosis: Tuberculosis antigens include, but are not limited to, lipoproteins, LPS, BCG antigens, a fusion protein of antigen 85B (Ag85B), ESAT-6 optionally formulated in cationic lipid vesicles, Mycobacterium tuberculosis (Mtb) isocitrate dehydrogenase associated antigens, and MPT51 antigens.
Rickettsia: Antigens include, but are not limited to, outer membrane proteins, including the outer membrane protein A and/or B (OmpB), LPS, and surface protein antigen (SPA).
Listeria monocytogenes: Bacterial antigens include, but are not limited to, those derived from Listeria monocytogenes.
Chlamydia pneumoniae: Antigens include, but are not limited to, those identified in WO 02/02606.
Vibrio cholerae: Antigens include, but are not limited to, proteinase antigens, LPS, particularly lipopolysaccharides of Vibrio cholerae II, O1 Inaba O-specific polysaccharides, V. cholera O139, antigens of IEM108 vaccine and Zonula occludens toxin (Zot).
Salmonella typhi (typhoid fever): Antigens include, but are not limited to, capsular polysaccharides preferably conjugates (Vi, i.e. vax-TyVi).
Borrelia burgdorferi (Lyme disease): Antigens include, but are not limited to, lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein, VlsE Antigenic Variation Protein.
Porphyromonas gingivalis: Antigens include, but are not limited to, P. gingivalis outer membrane protein (OMP).
Klebsiella: Antigens include, but are not limited to, an OMP, including OMP A, or a polysaccharide optionally conjugated to tetanus toxoid.

Other bacterial antigens used in the immunogenic compositions provided herein include, but are not limited to, capsular antigens, polysaccharide antigens or protein antigens of any of the above. Other bacterial antigens used in the immunogenic compositions provided herein include, but are not limited to, an outer membrane vesicle (OMV) preparation. Additionally, other bacterial antigens used in the immunogenic compositions provided herein include, but are not limited to, live, attenuated, and/or purified versions of any of the aforementioned bacteria. In certain embodiments, the bacterial antigens used in the immunogenic compositions provided herein are derived from gram-negative, while in other embodiments they are derived from gram-positive bacteria. In certain embodiments, the bacterial antigens used in the immunogenic compositions provided herein are derived from aerobic bacteria, while in other embodiments they are derived from anaerobic bacteria.

In certain embodiments, any of the above bacterial-derived saccharides (polysaccharides, LPS, LOS or oligosaccharides) are conjugated to another agent or antigen, such as a carrier protein (for example CRM₁₉₇). In certain embodiments, such conjugations are direct conjugations effected by reductive amination of carbonyl moieties on the saccharide to amino groups on the protein. In other embodiments, the saccharides are conjugated through a linker, such as, with succinamide or other linkages provided in Bioconjugate Techniques, 1996 and CRC, Chemistry of Protein Conjugation and Cross-Linking, 1993.

In certain embodiments useful for the treatment or prevention of Neisseria infection and related diseases and disorders, recombinant proteins from N. meningitidis for use in the immunogenic compositions provided herein may be found in WO99/24578, WO99/36544, WO99/57280, WO00/22430, WO96/29412, WO01/64920, WO03/020756, WO2004/048404, and WO2004/032958. Such antigens may be used alone or in combinations. Where multiple purified proteins are combined then it is helpful to use a mixture of 10 or fewer (e.g. 9, 8, 7, 6, 5, 4, 3, 2) purified antigens.

A particularly useful combination of antigens for use in the immunogenic compositions provided herein is disclosed in Giuliani et al. (2006) Proc Nail Acad Sci USA 103(29):10834-9 and WO2004/032958, and so an immunogenic composition may include 1, 2, 3, 4 or 5 of: (1) a ‘NadA’ protein (aka GNA1994 and NMB1994); (2) a ‘fHBP’ protein (aka ‘741’, LP2086, GNA1870, and NMB1870); (3) a ‘936’ protein (aka GNA2091 and NMB2091); (4) a ‘953’ protein (aka GNA1030 and NMB1030); and (5) a ‘287’ protein (aka GNA2132 and NMB2132). Other possible antigen combinations may comprise a transferrin binding protein (e.g. TbpA and/or TbpB) and an Hsf antigen. Other possible purified antigens for use in the compositions provided herein include proteins comprising one of the following amino acid sequences: SEQ ID NO:650 from WO99/24578; SEQ ID NO:878 from WO99/24578; SEQ ID NO:884 from WO99/24578; SEQ ID NO:4 from WO99/36544; SEQ ID NO:598 from WO99/57280; SEQ ID NO:818 from WO99/57280; SEQ ID NO:864 from WO99/57280; SEQ ID NO:866 from WO99/57280; SEQ ID NO:1196 from WO99/57280; SEQ ID NO:1272 from WO99/57280; SEQ ID NO:1274 from WO99/57280; SEQ ID NO:1640 from WO99/57280; SEQ ID NO:1788 from WO99/57280; SEQ ID NO:2288 from WO99/57280; SEQ ID NO:2466 from WO99/57280; SEQ ID NO:2554 from WO99/57280; SEQ ID NO:2576 from WO99/57280; SEQ ID NO:2606 from WO99/57280; SEQ ID NO:2608 from WO99/57280; SEQ ID NO:2616 from WO99/57280; SEQ ID NO:2668 from WO99/57280; SEQ ID NO:2780 from WO99/57280; SEQ ID NO:2932 from WO99/57280; SEQ ID NO:2958 from WO99/57280; SEQ ID NO:2970 from WO99/57280; SEQ ID NO:2988 from WO99/57280 (each of the forgoing amino acid sequences is hereby incorporated by reference from the cited document), or a polypeptide comprising an amino acid sequence which: (a) has 50% or more identity (e.g., 60%, 70%, 80%, 90%, 95%, 99% or more) to said sequences; and/or (b) comprises a fragment of at least n consecutive amino acids from said sequences, wherein n is 7 or more (e.g., 8, 10, 12, 14, 16, 18, 20, 25, 30, 35, 40, 50, 60, 70, 80, 90, 100, 150, 200, 250 or more). Preferred fragments for (b) comprise an epitope from the relevant sequence. More than one (e.g., 2, 3, 4, 5, 6) of these polypeptides may be included in the immunogenic compositions.

The fHBP antigen falls into three distinct variants (WO2004/048404). An N. meningitidis serogroup vaccine based upon the immunogenic compositions disclosed herein utilizing one of the compounds disclosed herein may include a single fHBP variant, but is will usefully include an fHBP from each of two or all three variants. Thus the composition may include a combination of two or three different purified fHBPs, selected from: (a) a first protein, comprising an amino acid sequence having at least a % sequence identity to SEQ ID NO: 9 and/or comprising an amino acid sequence consisting of a fragment of at least x contiguous amino acids from SEQ ID NO: 9; (b) a second protein, comprising an amino acid sequence having at least b % sequence identity to SEQ ID NO: 10 and/or comprising an amino acid sequence consisting of a fragment of at least y contiguous amino acids from SEQ ID NO: 10; and/or (c) a third protein, comprising an amino acid sequence having at least c % sequence identity to SEQ ID NO: 11 and/or comprising an amino acid sequence consisting of a fragment of at least z contiguous amino acids from SEQ ID NO: 11.

SEQ ID NO: 9
VAADIGAGLADALTAPLDHKDKGLQSLTLDQSVRKNEKLKLAAQGAEKTY
GNGDSLNTGKLKNDKVSRFDFIRQIEVDGQLITLESGEFQVYKQSHSALT
AFQTEQIQDSEHSGKMVAKRQFRIGDIAGEHTSFDKLPEGGRATYRGTAF
GSDDAGGKLTYTIDFAAKQGNGKIEHLKSPELNVDLAAADIKPDGKRHAV
ISGSVLYNQAEKGSYSLGIFGGKAQEVAGSAEVKTVNGIRHIGLAAKQ
SEQ ID NO: 10
VAADIGAGLADALTAPLDHKDKSLQSLTLDQSVRKNEKLKLAAQGAEKTY
GNGDSLNTGKLKNDKVSRFDFIRQIEVDGQLITLESGEFQIYKQDHSAVV
ALQIEKINNPDKIDSLINQRSFLVSGLGGEHTAFNQLPDGKAEYHGKAFS
SDDAGGKLTYTIDFAAKQGHGKIEHLKTPEQNVELAAAELKADEKSHAVI
LGDTRYGSEEKGTYHLALFGDRAQEIAGSATVKIGEKVHEIGIAGKQ
SEQ ID NO: 11
VAADIGTGLADALTAPLDHKDKGLKSLTLEDSIPQNGTLTLSAQGAEKTF
KAGDKDNSLNTGKLKNDKISRFDFVQKIEVDGQTITLASGEFQIYKQNHS
AVVALQIEKINNPDKTDSLINQRSFLVSGLGGEHTAFNQLPGGKAEYHGK
AFSSDDPNGRLHYSIDFTKKQGYGRIEHLKTLEQNVELAAAELKADEKSH
AVILGDTRYGSEEKGTYHLALFGDRAQEIAGSATVKIGEKVHEIGIAGKQ.

The value of a is at least 85, e.g., 86, 87, 88, 89, 90, 91, 92, 93, 94, 95, 96, 97, 98, 99, 99.5, or more. The value of b is at least 85, e.g., 86, 87, 88, 89, 90, 91, 92, 93, 94, 95, 96, 97, 98, 99, 99.5, or more. The value of c is at least 85, e.g., 86, 87, 88, 89, 90, 91, 92, 93, 94, 95, 96, 97, 98, 99, 99.5, or more. The values of a, b and c are not intrinsically related to each other.

The value of x is at least 7, e.g., 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7, e.g., 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7, e.g., 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z are not intrinsically related to each other.

In some embodiments, the immunogenic compositions as disclosed herein will include fHBP protein(s) that are lipidated, e.g., at a N-terminal cysteine. In other embodiments they will not be lapidated.

### Bacterial Vesicle Antigens

The immunogenic compositions as disclosed herein may include outer membrane vesicles. Such outer membrane vesicles may be obtained from a wide array of pathogenic bacteria and used as antigenic components of the immunogenic compositions as disclosed herein. Vesicles for use as antigenic components of such immunogenic compositions include any proteoliposomic vesicle obtained by disrupting a bacterial outer membrane to form vesicles therefrom that include protein components of the outer membrane. Thus the term includes OMVs (sometimes referred to as ‘blebs’), microvesicles (MVs, see, e.g., WO02/09643) and ‘native OMVs’ (‘NOMVs’ see, e.g., Katial et al. (2002) Infect. Immun. 70:702-707) Immunogenic compositions as disclosed herein that include vesicles from one or more pathogenic bacteria can be used in the treatment or prevention of infection by such pathogenic bacteria and related diseases and disorders.

MVs and NOMVs are naturally-occurring membrane vesicles that form spontaneously during bacterial growth and are released into culture medium. MVs can be obtained by culturing bacteria such as Neisseria in broth culture medium, separating whole cells from the smaller MVs in the broth culture medium (e.g., by filtration or by low-speed centrifugation to pellet only the cells and not the smaller vesicles), and then collecting the MVs from the cell-depleted medium (e.g., by filtration, by differential precipitation or aggregation of MVs, by high-speed centrifugation to pellet the MVs). Strains for use in production of MVs can generally be selected on the basis of the amount of MVs produced in culture (see, e.g., U.S. Pat. No. 6,180,111 and WO01/34642 describing Neisseria with high MV production).

OMVs are prepared artificially from bacteria, and may be prepared using detergent treatment (e.g., with deoxycholate), or by non detergent means (see, e.g., WO04/019977). Methods for obtaining suitable OMV preparations are well known in the art. Techniques for forming OMVs include treating bacteria with a bile acid salt detergent (e.g., salts of lithocholic acid, chenodeoxycholic acid, ursodeoxycholic acid, deoxycholic acid, cholic acid, ursocholic acid, etc., with sodium deoxycholate (EP0011243 and Fredriksen et al. (1991) NIPH Ann. 14(2):67-80) being preferred for treating Neisseria) at a pH sufficiently high not to precipitate the detergent (see, e.g., WO01/91788). Other techniques may be performed substantially in the absence of detergent (see, e.g., WO04/019977) using techniques such as sonication, homogenisation, microfluidisation, cavitation, osmotic shock, grinding, French press, blending, etc. Methods using no or low detergent can retain useful antigens such as NspA in Neisserial OMVs. Thus a method may use an OMV extraction buffer with about 0.5% deoxycholate or lower, e.g., about 0.2%, about 0.1%, <0.05% or zero.

A useful process for OMV preparation is described in WO05/004908 and involves ultrafiltration on crude OMVs, rather than instead of high speed centrifugation. The process may involve a step of ultracentrifugation after the ultrafiltration takes place.

Vesicles can be prepared from any pathogenic strain such as Neisseria minigtidis for use with the invention. Vessicles from Neisserial meningitidis serogroup B may be of any serotype (e.g., 1, 2a, 2b, 4, 14, 15, 16, etc.), any serosubtype, and any immunotype (e.g., L1; L2; L3; L3,3,7; L10; etc.). The meningococci may be from any suitable lineage, including hyperinvasive and hypervirulent lineages, e.g., any of the following seven hypervirulent lineages: subgroup I; subgroup III; subgroup IV 1; ET 5 complex; ET 37 complex; A4 cluster; lineage 3. These lineages have been defined by multilocus enzyme electrophoresis (MLEE), but multilocus sequence typing (MLST) has also been used to classify meningococci, e.g., the ET 37 complex is the ST 11 complex by MLST, the ET 5 complex is ST-32 (ET-5), lineage 3 is ST 41/44, etc. Vesicles can be prepared from strains having one of the following subtypes: P1.2; P1.2,5; P1.4; P1.5; P1.5,2; P1.5,c; P1.5c, 10; P1.7,16; P1.7,16b; P1.7h, 4; P1.9; P1.15; P1.9,15; P1.12,13; P1.13; P1.14; P1.21,16; P1.22,14.

Vesicles included in the immunogenic compositions disclosed herein may be prepared from wild type pathogenic strains such as N. meningitidis strains or from mutant strains. By way of example, WO98/56901 discloses preparations of vesicles obtained from N. meningitidis with a modified fur gene. WO02/09746 teaches that nspA expression should be up regulated with concomitant porA and cps knockout. Further knockout mutants of N. meningitidis for OMV production are disclosed in WO02/0974, WO02/062378, and WO04/014417. WO06/081259 discloses vesicles in which fHBP is upregulated. Claassen et al. (1996) 14(10):1001-8, disclose the construction of vesicles from strains modified to express six different PorA subtypes. Mutant Neisseria with low endotoxin levels, achieved by knockout of enzymes involved in LPS biosynthesis, may also be used (see, e.g., WO99/10497 and Steeghs et al. (2001) i20:6937-6945). These or others mutants can all be used with the invention.

Thus N. meningitidis serogroup B strains included in the immunogenic compositions disclosed herein may in some embodiments express more than one PorA subtype. Six valent and nine valent PorA strains have previously been constructed. The strain may express 2, 3, 4, 5, 6, 7, 8 or 9 of PorA subtypes: P1.7,16; P1.5-1, 2-2; P1,19,15-1; P1.5-2,10; P1.12 1,13; P1.7-2,4; P1.22,14; P1.7-1,1 and/or P1.18-1,3,6. In other embodiments a strain may have been down regulated for PorA expression, e.g., in which the amount of PorA has been reduced by at least 20% (e.g., >30%, >40%, >50%, >60%, >70%, >80%, >90%, >95%, etc.), or even knocked out, relative to wild type levels (e.g., relative to strain H44/76, as disclosed in WO03/105890).

In some embodiments N. meningitidis serogroup B strains may over express (relative to the corresponding wild-type strain) certain proteins. For instance, strains may over express NspA, protein 287 (WO01/52885—also referred to as NMB2132 and GNA2132), one or more fHBP (WO06/081259 and U.S. Pat. Pub. 2008/0248065—also referred to as protein 741, NMB1870 and GNA1870), TbpA and/or TbpB (WO00/25811), Cu,Zn-superoxide dismutase (WO00/25811), etc.

In some embodiments N. meningitidis serogroup B strains may include one or more of the knockout and/or over expression mutations. Preferred genes for down regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB (WO01/09350); (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB (WO02/09746); (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB (WO02/062378); and (d) CtrA, CtrB, CtrD, FrpB, OpA, OpC, PilC, PorB, SiaD, SynA, SynB, and/or SynC (WO04/014417).

Where a mutant strain is used, in some embodiments it may have one or more, or all, of the following characteristics: (i) down regulated or knocked-out LgtB and/or GalE to truncate the meningococcal LOS; (ii) up regulated TbpA; (iii) up regulated Hsf; (iv) up regulated Omp85; (v) up regulated LbpA; (vi) up regulated NspA; (vii) knocked-out PorA; (viii) down regulated or knocked-out FrpB; (ix) down regulated or knocked-out Opa; (x) down regulated or knocked-out Opc; (xii) deleted cps gene complex. A truncated LOS can be one that does not include a sialyl-lacto-N-neotetraose epitope, e.g., it might be a galactose-deficient LOS. The LOS may have no a chain.

If LOS is present in a vesicle then it is possible to treat the vesicle so as to link its LOS and protein components (“intra-bleb” conjugation (WO04/014417)).

The immunogenic compositions as disclosed herein may include mixtures of vesicles from different strains. By way of example, WO03/105890 discloses vaccine comprising multivalent meningococcal vesicle compositions, comprising a first vesicle derived from a meningococcal strain with a serosubtype prevalent in a country of use, and a second vesicle derived from a strain that need not have a serosubtype prevent in a country of use. WO06/024946 discloses useful combinations of different vesicles. A combination of vesicles from strains in each of the L2 and L3 immunotypes may be used in some embodiments.

Vesicle-based antigens can be prepared from N. meningitidis serogroups other than serogroup B (e.g., WO01/91788 discloses a process for serogroup A). The immunogenic compositions disclosed herein accordingly can include vesicles prepared serogroups other than B (e.g. A, C, W135 and/or Y) and from bacterial pathogens other than Neisseria.

### Viral Antigens

Viral antigens suitable for use in the immunogenic compositions provided herein include, but are not limited to, inactivated (or killed) virus, attenuated virus, split virus formulations, purified subunit formulations, viral proteins which may be isolated, purified or derived from a virus, and Virus Like Particles (VLPs). In certain embodiments, viral antigens are derived from viruses propagated on cell culture or other substrate. In other embodiments, viral antigens are expressed recombinantly. In certain embodiments, viral antigens preferably include epitopes which are exposed on the surface of the virus during at least one stage of its life cycle. Viral antigens are preferably conserved across multiple serotypes or isolates. Viral antigens suitable for use in the immunogenic compositions provided herein include, but are not limited to, antigens derived from one or more of the viruses set forth below as well as the specific antigens examples identified below.
Orthomyxovirus: Viral antigens include, but are not limited to, those derived from an Orthomyxovirus, such as Influenza A, B and C. In certain embodiments, orthomyxovirus antigens are selected from one or more of the viral proteins, including hemagglutinin (HA), neuraminidase (NA), nucleoprotein (NP), matrix protein (M1), membrane protein (M2), one or more of the transcriptase components (PB1, PB2 and PA). In certain embodiments the viral antigen include HA and NA. In certain embodiments, the influenza antigens are derived from interpandemic (annual) flu strains, while in other embodiments, the influenza antigens are derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).
Paramyxoviridae viruses: Viral antigens include, but are not limited to, those derived from Paramyxoviridae viruses, such as Pneumoviruses (RSV), Paramyxoviruses (PIV), Metapneumovirus and Morbilliviruses (Measles).
Pneumovirus: Viral antigens include, but are not limited to, those derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. In certain embodiments, pneumovirus antigens are selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. In other embodiments, pneumovirus antigens include F, G and M. In certain embodiments, pneumovirus antigens are also formulated in or derived from chimeric viruses, such as, by way of example only, chimeric RSV/PIV viruses comprising components of both RSV and PIV.
Paramyxovirus: Viral antigens include, but are not limited to, those derived from a Paramyxovirus, such as Parainfluenza virus types 1-4 (PIV), Mumps, Sendai viruses, Simian virus 5, Bovine parainfluenza virus, Nipahvirus, Henipavirus and Newcastle disease virus. In certain embodiments, the Paramyxovirus is PIV or Mumps. In certain embodiments, paramyxovirus antigens are selected from one or more of the following proteins: Hemagglutinin-Neuraminidase (HN), Fusion proteins F1 and F2, Nucleoprotein (NP), Phosphoprotein (P), Large protein (L), and Matrix protein (M). In other embodiments, paramyxovirus proteins include HN, F1 and F2. In certain embodiments, paramyxovirus antigens are also formulated in or derived from chimeric viruses, such as, by way of example only, chimeric RSV/PIV viruses comprising components of both RSV and PIV. Commercially available mumps vaccines include live attenuated mumps virus, in either a monovalent form or in combination with measles and rubella vaccines (MMR). In other embodiments, the Paramyxovirus is Nipahvirus or Henipavirus and the antigens are selected from one or more of the following proteins: Fusion (F) protein, Glycoprotein (G) protein, Matrix (M) protein, Nucleocapsid (N) protein, Large (L) protein and Phosphoprotein (P).

Poxyiridae: Viral antigens include, but are not limited to, those derived from Orthopoxvirus such as Variola vera, including but not limited to, Variola major and Variola minor.
Metapneumovirus: Viral antigens include, but are not limited to, Metapneumovirus, such as human metapneumovirus (hMPV) and avian metapneumoviruses (aMPV). In certain embodiments, metapneumovirus antigens are selected from one or more of the following proteins, including surface proteins Fusion (F), Glycoprotein (G) and Small Hydrophobic protein (SH), matrix proteins M and M2, nucleocapsid proteins N, P and L. In other embodiments, metapneumovirus antigens include F, G and M. In certain embodiments, metapneumovirus antigens are also formulated in or derived from chimeric viruses.
Morbillivirus: Viral antigens include, but are not limited to, those derived from a Morbillivirus, such as Measles. In certain embodiments, morbillivirus antigens are selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).
Picornavirus: Viral antigens include, but are not limited to, those derived from Picornaviruses, such as Enteroviruses, Rhinoviruses, Heparnavirus, Parechovirus, Cardioviruses and Aphthoviruses. In certain embodiments, the antigens are derived from Enteroviruses, while in other embodiments the enterovirus is Poliovirus. In still other embodiments, the antigens are derived from Rhinoviruses. In certain embodiments, the antigens are formulated into virus-like particles (VLPs).
Enterovirus: Viral antigens include, but are not limited to, those derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. In certain embodiments, the antigens are derived from Enteroviruses, while in other embodiments the enterovirus is Poliovirus. In certain embodiments, the enterovirus antigens are selected from one or more of the following Capsid proteins VP0, VP1, VP2, VP3 and VP4. Commercially available polio vaccines include Inactivated Polio Vaccine (IPV) and Oral poliovirus vaccine (OPV). In certain embodiments, the antigens are formulated into virus-like particles.
Bunyavirus: Viral antigens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.
Rhinovirus: Viral antigens include, but are not limited to, those derived from rhinovirus. In certain embodiments, the rhinovirus antigens are selected from one or more of the following Capsid proteins: VP0, VP1, VP2, VP2 and VP4. In certain embodiments, the antigens are formulated into virus-like particles (VLPs).
Heparnavirus: Viral antigens include, but are not limited to, those derived from a Heparnavirus, such as, by way of example only, Hepatitis A virus (HAV). Commercially available HAV vaccines include inactivated HAV vaccine.
Togavirus: Viral antigens include, but are not limited to, those derived from a Togavirus, such as a Rubivirus, an Alphavirus, or an Arterivirus. In certain embodiments, the antigens are derived from Rubivirus, such as by way of example only, Rubella virus. In certain embodiments, the togavirus antigens are selected from E1, E2, E3, C, NSP-1, NSPO-2, NSP-3 or NSP-4. In certain embodiments, the togavirus antigens are selected from E1, E2 or E3. Commercially available Rubella vaccines include a live cold-adapted virus, typically in combination with mumps and measles vaccines (MMR).
Flavivirus: Viral antigens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus. In certain embodiments, the flavivirus antigens are selected from PrM, M, C, E, NS-1, NS-2a, NS2b, NS3, NS4a, NS4b, and NS5. In certain embodiments, the flavivirus antigens are selected from PrM, M and E. Commercially available TBE vaccine includes inactivated virus vaccines. In certain embodiments, the antigens are formulated into virus-like particles (VLPs).
Pestivirus: Viral antigens include, but are not limited to, those derived from a Pestivirus, such as Bovine viral diarrhea (BVDV), Classical swine fever (CSFV) or Border disease (BDV).
Hepadnavirus: Viral antigens include, but are not limited to, those derived from a Hepadnavirus, such as Hepatitis B virus. In certain embodiments, the hepadnavirus antigens are selected from surface antigens (L, M and S), core antigens (HBc, HBe). Commercially available HBV vaccines include subunit vaccines comprising the surface antigen S protein.
Hepatitis C virus: Viral antigens include, but are not limited to, those derived from a Hepatitis C virus (HCV). In certain embodiments, the HCV antigens are selected from one or more of E1, E2, E1/E2, NS345 polyprotein, NS 345-core polyprotein, core, and/or peptides from the nonstructural regions. In certain embodiments, the Hepatitis C virus antigens include one or more of the following: HCV E1 and or E2 proteins, E1/E2 heterodimer complexes, core proteins and non-structural proteins, or fragments of these antigens, wherein the non-structural proteins can optionally be modified to remove enzymatic activity but retain immunogenicity. In certain embodiments, the antigens are formulated into virus-like particles (VLPs).
Rhabdovirus: Viral antigens include, but are not limited to, those derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells.
Caliciviridae; Viral antigens include, but are not limited to, those derived from Calciviridae, such as Norwalk virus, and Norwalk-like Viruses, such as Hawaii Virus and Snow Mountain Virus. In certain embodiments, the antigens are formulated into virus-like particles (VLPs).
Coronavirus: Viral antigens include, but are not limited to, those derived from a Coronavirus, SARS, Human respiratory coronavirus, Avian infectious bronchitis (IBV), Mouse hepatitis virus (MHV), and Porcine transmissible gastroenteritis virus (TGEV). In certain embodiments, the coronavirus antigens are selected from spike (S), envelope (E), matrix (M), nucleocapsid (N), and Hemagglutinin-esterase glycoprotein (HE). In certain embodiments, the coronavirus antigen is derived from a SARS virus. In certain embodiments, the coronavirus is derived from a SARS viral antigen as described in WO 04/92360.
Retrovirus: Viral antigens include, but are not limited to, those derived from a Retrovirus, such as an Oncovirus, a Lentivirus or a Spumavirus. In certain embodiments, the oncovirus antigens are derived from HTLV-1, HTLV-2 or HTLV-5. In certain embodiments, the lentivirus antigens are derived from HIV-1 or HIV-2. In certain embodiments, the antigens are derived from HIV-1 subtypes (or clades), including, but not limited to, HIV-1 subtypes (or clades) A, B, C, D, F, G, H, J. K, O. In other embodiments, the antigens are derived from HIV-1 circulating recombinant forms (CRFs), including, but not limited to, A/B, A/E, A/G, A/G/I, etc. In certain embodiments, the retrovirus antigens are selected from gag, pol, env, tax, tat, rex, rev, nef, vif, vpu, and vpr. In certain embodiments, the HIV antigens are selected from gag (p24gag and p55gag), env (gp160 and gp41), pol, tat, nef, rev vpu, miniproteins, (preferably p55 gag and gp140v delete). In certain embodiments, the HIV antigens are derived from one or more of the following strains: HIV_{IIIb}, HIV_{SF2}, HIV_{LAV}, HIV_{LAI}, HIV_{MN}, HIV-1_{CM235}, HIV-1_{US4}, HIV-1_{SF162}, HIV-1_{TV1}, HIV-1_{MJ4}. In certain embodiments, the antigens are derived from endogenous human retroviruses, including, but not limited to, HERV-K (“old” HERV-K and “new” HERV-K).
Reovirus: Viral antigens include, but are not limited to, those derived from a Reovirus, such as an Orthoreovirus, a Rotavirus, an Orbivirus, or a Coltivirus. In certain embodiments, the reovirus antigens are selected from structural proteins λ1, λ2, λ3, μ1, μ2, σ1, σ2, or σ3, or nonstructural proteins σNS, μNS, or σ1s. In certain embodiments, the reovirus antigens are derived from a Rotavirus. In certain embodiments, the rotavirus antigens are selected from VP1, VP2, VP3, VP4 (or the cleaved product VP5 and VP8), NSP1, VP6, NSP3, NSP2, VP7, NSP4, or NSP5. In certain embodiments, the rotavirus antigens include VP4 (or the cleaved product VP5 and VP8), and VP7.
Parvovirus: Viral antigens include, but are not limited to, those derived from a Bocavirus and Parvovirus, such as Parvovirus B19. In certain embodiments, the Parvovirus antigens are selected from VP-1, VP-2, VP-3, NS-1 and NS-2. In certain embodiments, the Parvovirus antigen is capsid protein VP1 or VP-2. In certain embodiments, the antigens are formulated into virus-like particles (VLPs).
Delta hepatitis virus (HD V): Viral antigens include, but are not limited to, those derived from HDV, particularly δ-antigen from HDV.
Hepatitis E virus (HEV): Viral antigens include, but are not limited to, those derived from HEV.
Hepatitis G virus (HGV): Viral antigens include, but are not limited to, those derived from HGV.
Human Herpesvirus Viral antigens include, but are not limited to, those derived from a Human Herpesvirus, such as, by way of example only, Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). In certain embodiments, the Human Herpesvirus antigens are selected from immediate early proteins (α), early proteins (β), and late proteins (γ). In certain embodiments, the HSV antigens are derived from HSV-1 or HSV-2 strains. In certain embodiments, the HSV antigens are selected from glycoproteins gB, gC, gD and gH, fusion protein (gB), or immune escape proteins (gC, gE, or gI). In certain embodiments, the VZV antigens are selected from core, nucleocapsid, tegument, or envelope proteins. A live attenuated VZV vaccine is commercially available. In certain embodiments, the EBV antigens are selected from early antigen (EA) proteins, viral capsid antigen (VCA), and glycoproteins of the membrane antigen (MA). In certain embodiments, the CMV antigens are selected from capsid proteins, envelope glycoproteins (such as gB and gH), and tegument proteins. In other embodiments, CMV antigens may be selected from one or more of the following proteins: pp65, IE1, gB, gD, gH, gL, gM, gN, gO, UL128, UL129, gUL130, UL150, UL131, UL33, UL78, US27, US28, RL5A, RL6, RL10, RL11, RL12, RL13, UL1, UL2, UL4, UL5, UL6, UL7, UL8, UL9, UL10, UL11, UL14, UL15A, UL16, UL17, UL18, UL22A, UL38, UL40, UL41A, UL42, UL116, UL119, UL120, UL121, UL124, UL132, UL147A, UL148, UL142, UL144, UL141, UL140, UL135, UL136, UL138, UL139, UL133, UL135, UL148A, UL148B, UL148C, UL148D, US2, US3, US6, US7, US8, US9, US10, US11, US12, US13, US14, US15, US16, US17, US18, US19, US20, US21, US29, US30 and US34A. CMV antigens may also be fusions of one or more CMV proteins, such as, by way of example only, pp65/IE1 (Reap et al., Vaccine (2007) 25:7441-7449). In certain embodiments, the antigens are formulated into virus-like particles (VLPs).
Papovaviruses: Antigens include, but are not limited to, those derived from Papovaviruses, such as Papillomaviruses and Polyomaviruses. In certain embodiments, the Papillomaviruses include HPV serotypes 1, 2, 4, 5, 6, 8, 11, 13, 16, 18, 31, 33, 35, 39, 41, 42, 47, 51, 57, 58, 63 and 65. In certain embodiments, the HPV antigens are derived from serotypes 6, 11, 16 or 18. In certain embodiments, the HPV antigens are selected from capsid proteins (L1) and (L2), or E1-E7, or fusions thereof. In certain embodiments, the HPV antigens are formulated into virus-like particles (VLPs). In certain embodiments, the Polyomyavirus viruses include BK virus and JK virus. In certain embodiments, the Polyomavirus antigens are selected from VP1, VP2 or VP3.
Adenovirus: Antigens include those derived from Adenovirus. In certain embodiments, the Adenovirus antigens are derived from Adenovirus serotype 36 (Ad-36). In certain embodiments, the antigen is derived from a protein or peptide sequence encoding an Ad-36 coat protein or fragment thereof (WO 2007/120362).
Arenavirus: Viral antigens include, but are not limited to, those derived from Arenaviruses.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the immunogenic compositions provided herein.

### Fungal Antigens

Fungal antigens for use in the immunogenic compositions provided herein include, but are not limited to, those derived from one or more of the fungi set forth below.

Fungal antigens are derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; and
Fungal pathogens are derived from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In certain embodiments, the process for producing a fungal antigen includes a method wherein a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

### Protazoan Antigens/Pathogens

Protazoan antigens/pathogens for use in the immunogenic compositions provided herein include, but are not limited to, those derived from one or more of the following protozoa: Entamoeba histolytica, Giardia lambli, Cryptosporidium parvum, Cyclospora cayatanensis and Toxoplasma.

### Plant Antigens/Pathogens

Plant antigens/pathogens for use in the immunogenic compositions provided herein include, but are not limited to, those derived from Ricinus communis.

### STD Antigens

In certain embodiments, the immunogenic compositions provided herein include one or more antigens derived from a sexually transmitted disease (STD). In certain embodiments, such antigens provide for prophylactis for STD's such as chlamydia, genital herpes, hepatitis (such as HCV), genital warts, gonorrhea, syphilis and/or chancroid. In other embodiments, such antigens provide for therapy for STD's such as chlamydia, genital herpes, hepatitis (such as HCV), genital warts, gonorrhea, syphilis and/or chancroid. Such antigens are derived from one or more viral or bacterial STD's. In certain embodiments, the viral STD antigens are derived from HIV, herpes simplex virus (HSV-1 and HSV-2), human papillomavirus (HPV), and hepatitis (HCV). In certain embodiments, the bacterial STD antigens are derived from Neiserria gonorrhoeae, Chlamydia trachomatis, Treponema pallidum, Haemophilus ducreyi, E. coli, and Streptococcus agalactiae. Examples of specific antigens derived from these pathogens are described above.

### Respiratory Antigens

In certain embodiments, the immunogenic compositions provided herein include one or more antigens derived from a pathogen which causes respiratory disease. By way of example only, such respiratory antigens are derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). In certain embodiments, the respiratory antigens are derived from a bacteria which causes respiratory disease, such as, by way of example only, Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

### Pediatric Vaccine Antigen

In certain embodiments, the immunogenic compositions provided herein include one or more antigens suitable for use in pediatric subjects. Pediatric subjects are typically less than about 3 years old, or less than about 2 years old, or less than about 1 years old. Pediatric antigens are administered multiple times over the course of 6 months, 1, 2 or 3 years. Pediatric antigens are derived from a virus which may target pediatric populations and/or a virus from which pediatric populations are susceptible to infection. Pediatric viral antigens include, but are not limited to, antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitides, Streptococcus pyogenes (Group A Streptococcus), Moraxella catarrhalis, Bordetella pertussis, Staphylococcus aureus, Clostridium tetani (Tetanus), Cornynebacterium diphtheriae (Diphtheria), Haemophilus influenzae B (Hib), Pseudomonas aeruginosa, Streptococcus agalactiae (Group B Streptococcus), and E. coli. Examples of specific antigens derived from these pathogens are described above.

### Antigens Suitable for Use in Elderly or Immunocompromised Individuals

In certain embodiments, the immunogenic compositions provided herein include one or more antigens suitable for use in elderly or immunocompromised individuals. Such individuals may need to be vaccinated more frequently, with higher doses or with adjuvanted formulations to improve their immune response to the targeted antigens. Antigens which are targeted for use in Elderly or Immunocompromised individuals include antigens derived from one or more of the following pathogens: Neisseria meningitides, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Cornynebacterium diphtheriae (Diphtheria), Haemophilus influenzae B (Hib), Pseudomonas aeruginosa, Legionella pneumophila, Streptococcus agalactiae (Group B Streptococcus), Enterococcus faecalis, Helicobacter pylori, Chlamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

### Antigens Suitable for Use in Adolescent Vaccines

In certain embodiments, the immunogenic compositions provided herein include one or more antigens suitable for use in adolescent subjects. Adolescents are in need of a boost of a previously administered pediatric antigen. Pediatric antigens which are suitable for use in adolescents are described above. In addition, adolescents are targeted to receive antigens derived from an STD pathogen in order to ensure protective or therapeutic immunity before the beginning of sexual activity. STD antigens which are suitable for use in adolescents are described above.

### Tumor Antigens

In certain embodiments, a tumor antigen or cancer antigen is used in conjunction with the immunogenic compositions provided herein. In certain embodiments, the tumor antigens is a peptide-containing tumor antigens, such as a polypeptide tumor antigen or glycoprotein tumor antigens. In certain embodiments, the tumor antigen is a saccharide-containing tumor antigen, such as a glycolipid tumor antigen or a ganglioside tumor antigen. In certain embodiments, the tumor antigen is a polynucleotide-containing tumor antigen that expresses a polypeptide-containing tumor antigen, for instance, an RNA vector construct or a DNA vector construct, such as plasmid DNA.

Tumor antigens appropriate for the use in conjunction with the immunogenic compositions provided herein encompass a wide variety of molecules, such as (a) polypeptide-containing tumor antigens, including polypeptides (which can range, for example, from 8-20 amino acids in length, although lengths outside this range are also common), lipopolypeptides and glycoproteins, (b) saccharide-containing tumor antigens, including poly-saccharides, mucins, gangliosides, glycolipids and glycoproteins, and (c) polynucleotides that express antigenic polypeptides.

In certain embodiments, the tumor antigens are, for example, (a) full length molecules associated with cancer cells, (b) homologs and modified forms of the same, including molecules with deleted, added and/or substituted portions, and (c) fragments of the same. In certain embodiments, the tumor antigens are provided in recombinant form. In certain embodiments, the tumor antigens include, for example, class I-restricted antigens recognized by CD8+ lymphocytes or class II-restricted antigens recognized by CD4+ lymphocytes.

In certain embodiments, the tumor antigens include, but are not limited to, (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g., prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins are coupled to a carrier protein (e.g., MUC-1 are coupled to KLH); (ii) lipopolypeptides (e.g., MUC-1 linked to a lipid moiety); (iii) polysaccharides (e.g., Globo H synthetic hexasaccharide), which are coupled to a carrier proteins (e.g., to KLH), (iv) gangliosides such as GM2, GM12, GD2, GD3 (associated with, e.g., brain, lung cancer, melanoma), which also are coupled to carrier proteins (e.g., KLH).

In certain embodiments, the tumor antigens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

Polynucleotide-containing antigens used in conjunction with the immunogenic compositions provided herein include polynucleotides that encode polypeptide cancer antigens such as those listed above. In certain embodiments, the polynucleotide-containing antigens include, but are not limited to, DNA or RNA vector constructs, such as plasmid vectors (e.g., pCMV), which are capable of expressing polypeptide cancer antigens in vivo.

In certain embodiments, the tumor antigens are derived from mutated or altered cellular components. After alteration, the cellular components no longer perform their regulatory functions, and hence the cell may experience uncontrolled growth. Representative examples of altered cellular components include, but are not limited to ras, p53, Rb, altered protein encoded by the Wilms' tumor gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor.

Bacterial and viral antigens, may be used in conjunction with the compositions of the present invention for the treatment of cancer. In particular, carrier proteins, such as CRM₁₉₇, tetanus toxoid, or Salmonella typhimurium antigen may be used in conjunction/conjugation with compounds of the present invention for treatment of cancer. The cancer antigen combination therapies will show increased efficacy and bioavailability as compared with existing therapies.

Additional information on cancer or tumor antigens can be found, for example, in Moingeon (2001) Vaccine 19:1305-1326; Rosenberg (2001) Nature 411:380-384; Dermine et al. (2002) Brit. Med. Bull. 62:149-162; Espinoza-Delgado (2002) The Oncologist 7(suppl 3):20-33; Davis et al. (2003) J. Leukocyte Biol. 23:3-29; Van den Eynde et al. (1995) Curr. Opin. Immunol. 7:674-681; Rosenberg (1997) Immunol. Today 18:175-182; Offring a et al. (2000) Curr. Opin. Immunol. 2:576-582; Rosenberg (1999) Immunity 10:281-287; Sahin et al. (1997) Curr. Opin. Immunol. 9:709-716; Old et al. (1998) J. Exp. Med. 187:1163-1167; Chaux et al. (1999) J. Exp. Med. 189:767-778; Gold et al. (1965) J. Exp. Med. 122:467-468; Livingston et al. (1997) Cancer Immunol. Immunother. 45:1-6; Livingston et al. (1997) Cancer Immunol. Immunother. 45:10-19; Taylor-Papadimitriou (1997) Immunol. Today 18:105-107; Zhao et al. (1995) J. Exp. Med. 182:67-74; Theobald et al. (1995) Proc. Natl. Acad. Sci. USA 92:11993-11997; Gaudernack (1996) Immunotechnology 2:3-9; WO 91/02062; U.S. Pat. No. 6,015,567; WO 01/08636; WO 96/30514; U.S. Pat. No. 5,846,538; and U.S. Pat. No. 5,869,445.

### C. Targeting Ligands

According to one embodiment, the particle(s) formed by the amphipathic peptides and the lipids are capable of binding a target such as, for example, a receptor or a cell surface structure such as a cell marker. For example, the amphipathic peptides described above which are capable of mimicking properties of apolipoprotein A1 may be able to interact with the SRB-1 receptor and thus are suitable for a targeted delivery to cells carrying the SRB-1 receptor.

However, in order to be able to provide a targeted delivery of the composition to a target of choice (e.g. a body compartment, an organ, a cell type or a tumor), it is advantageous that the composition comprises a targeting ligand. A respective targeting ligand allows a targeted delivery of the composition to the target of choice, e.g. to a specific body compartment, organ, tissue or tumor. Furthermore, the targeting ligand may enable a target specific uptake into a cell of choice (e.g., an antigen presenting cell such as a dendritic cell, monocyte, macrophage, etc.). Various strategies can be used in order to provide a targeted delivery, such as for example targeting of the folate and asialoglycoprotein receptors, glucosaminoglycans and various receptors and markers expressed on tumor cells through strategies including but not limited to using binding molecules such as antibodies and antibody fragments specific for the respective target, anticalines, aptamers, small molecules, natural and non-natural carbohydrates, peptides and polypeptides as targeting ligands. Also lymphoid tissue may be targeted.

Several approaches may be employed by which a targeting ligand may be associated with the particle(s) formed by the amphipathic peptides and the lipids.

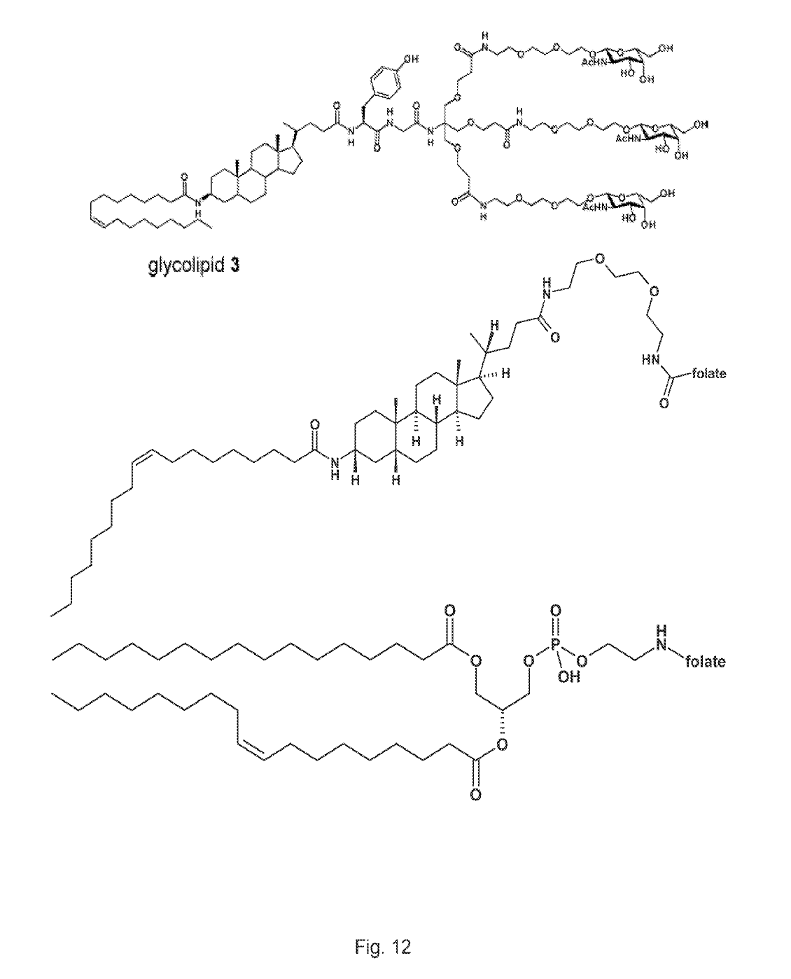

According to one embodiment, the targeting ligand comprises a lipophilic anchor. The targeting ligand is thus anchored via the respective lipophilic anchor to the particle as the lipophilic anchor inserts into the lipid core. The lipophilic anchor can be directly linked to the targeting ligand or by use of an appropriate linker structure. FIG. 12 shows certain lipidated targeting motifs useful for particle targeting.

According to a further embodiment, the targeting ligand is linked to the immunogenic species. This can be done by direct attachment/coupling or by use of appropriate linker groups. Also non-covalent associations are within the scope of the present application.

According to a further embodiment, the targeting ligand is attached or associated with at least one of the amphipathic peptides. This can be done for example by non-covalent or covalent attachment. Again, an appropriate linker group can be used.

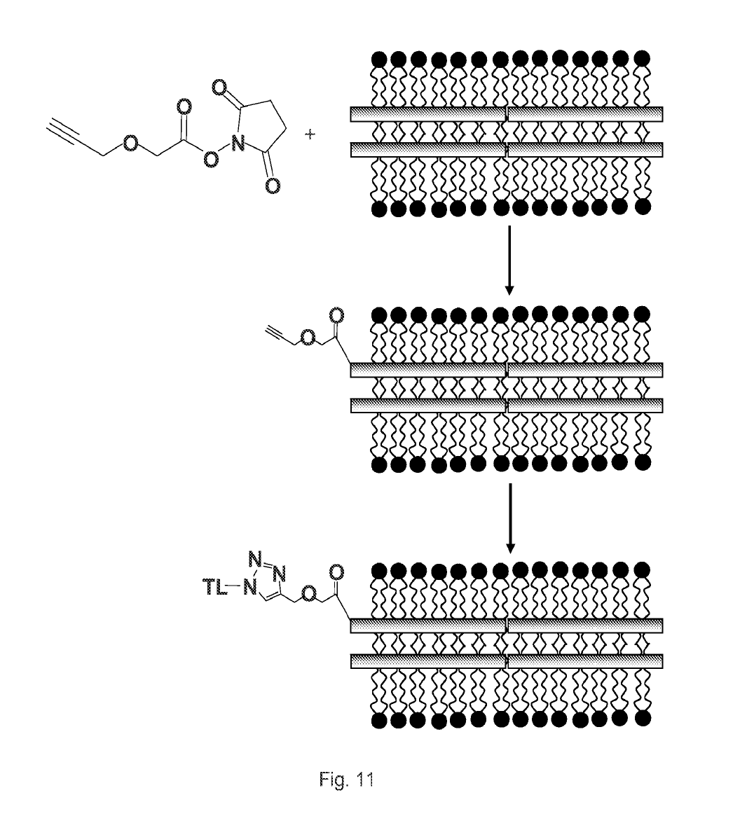

For example, FIG. 11 schematically shows an embodiment for functionalizing the amphipathic peptides of the invention. An amphipathic peptide is shown, wherein the lysine side chains are available and thus accessible for chemical modification. The lysine side chains are modified with an alkyne and thus provide an anchoring site for attaching a targeting ligand TL, in this case a targeting ligand with an azide functional group, which leads to the formation of the 1,2,3-triazole shown (e.g., via azide-alkyne Huisgen cycloaddition, which is a 1,3-dipolar cycloaddition between an azide and a terminal or internal alkyne to give a 1,2,3-triazole).

Also combinations of the above-described approaches are possible and within the scope of the present invention.

### D. Lipophilic Anchors

The lipophilic anchors that can be used to associate the targeting ligand, the capturing agent and/or the immunogenic species with the particle(s) formed by the amphipathic peptides and the lipids as described above, can be, for example, selected from the group consisting of (a) cholesterol, (b) hydrophobic fatty acids and (c) bile acid derivatives, among others.

Respective groups have been shown to be useful to achieve anchoring to the lipids of the particles according to the present invention. It has been shown that lipid anchors such as cholesterol are particularly useful for tightly anchoring the immunogenic species, the targeting ligand and/or the capturing agent to the particles. When a hydrophobic fatty acid is used, it is in particular useful if the lipophilic anchor is strongly hydrophobic and has, for example, at least one long alkyl and/or alkenyl chain having, for example, at least 18 carbon atoms. It is also possible to use bile acid derivatives comprising a hydrophobic group. For example, stearoyl, docosanyl and lithocholeic-oleoyl radicals are suitable lipophilic anchors.

According to one embodiment, the lipophilic anchor is attached to the targeting ligand, the capturing agent and/or the immunogenic species via a cleavable linker which comprises e.g. a disulfide bridge. This embodiment is in particular useful for anchoring the immunogenic species. Preferably, linkers are used which are acid cleavable. It is assumed that the immunogenic species associated via the lipophilic anchor to the particles is contained in the endosomes upon entering the target cell. The use of an acid-cleavable linker has the advantage that the linker is cleaved upon entering/processing in the endosome, thereby releasing the immunogenic species. This simplifies the release of the immunogenic species from the carrier particles. This embodiment also enables the use of a lipophilic anchor which binds particularly tightly to the lipids of the particles. A tight anchorage prevents undesired/unintentional detachment of the immunogenic species from the particles.

Examples of acid-labile and biodegradable linkers include those that contain a chemical group such as acetals, ketals, orthoesters, imines, hydrazones, oximes, esters, N-alkoxybezylimidazoles, enol ethers, enol esters, enamides, carbonates, maleamates, and others known to those skilled in the art. Another example is linkers containing peptide sequences known to be substrates for proteases.

### E. Compositions and Formulations

Also provided with the present invention is a composition, comprising particles of amphipathic peptides and lipids for use as a carrier for at least one immunogenic species. Respective particles are in particular useful for delivering at least one immunogenic species to a vertebrate subject, in particular a human. Delivery is preferably systemic or local. The details of the respective particles, including the nature of the amphipathic peptides, the lipids and the potential use of targeting ligands and anchoring moieties, is described in detail above and also applies to the composition according to the present application which can be used for transporting and delivering an immunogenic species. For delivery/transport, the particles comprising the amphipathic peptides and the lipids are mixed with the at least one immunogenic species in order to obtain a composition also comprising the immunogenic species to be delivered.

Also provided according to the present invention is a pharmaceutical composition which comprises a composition as is outlined above (e.g., a composition comprising particles of amphipathic peptides and lipids, which may either be loaded with an immunogenic species or not loaded) and one or more of a wide variety of supplemental components. The pharmaceutical composition may thus comprise one or more pharmaceutically acceptable excipients as supplemental components. For example, liquid vehicles such as water, saline, glycerol, polyethylene glycol, ethanol, etc. may be used. Other excipients, such as wetting or emulsifying agents, tonicity adjusting agents, biological buffering substances, and the like, may be present. A biological buffer can be virtually any species which is/are pharmacologically acceptable and which provide the formulation with the desired pH, i.e., a pH in the physiological range. Examples of buffered systems include phosphate buffered saline, Tris buffered saline, Hank's buffered saline, and the like. Depending on the final dosage form, other excipients known in the art can also be introduced, including binders, disintegrants, fillers (diluents), lubricants, glidants (flow enhancers), compression aids, sweeteners, flavors, preservatives, suspensing/dispersing agents, film formers/coatings, and so forth.

In certain embodiments, pharmaceutical compositions in accordance with the present invention are lyophilized.

In certain embodiments, pharmaceutical compositions in accordance with the present invention comprise at least one surfactant, at least one cryoprotective agent, or both. Examples of cryoprotective agents include polyols, carbohydrates and combinations thereof, among others. Examples of surfactants include non-ionic surfactants, cationic surfactants, anionic surfactants, and zwitterionic surfactants, among others. Surfactants and/or cryoprotective agents may be added, for example, to allow the lyophilized compositions to be resuspended without an unacceptable increase in particle size (e.g., without significant undesired aggregation).

Also provided are methods for producing compositions according to the present application, comprising amphipathic peptides, lipids and at least one immunogenic species.

For example, a stock solution of the amphipathic peptide in a suitable solvent such as methanol may be prepared. A stock solution of the lipid may also be prepared in a suitable solvent such as methanol. Typical weight ratios of peptide to lipid range from 1:0.5 to 1:5, more typically, 1:1 to 1:2, among other values. As is outlined above, the lipids form particles with the peptides which are believed to mimic lipoprotein structures. For mixing the lipids with the peptides, the mixture can be vortexed in order to thoroughly mix the lipids with the peptides. Where the peptides and/or the lipids are comprised in alcohol such as methanol, the alcohol should be evaporated after mixing the components. A film formed by the peptides and lipids is dried and is afterwards hydrated with a suitable liquid (e.g., saline or a buffered solution such as phosphate buffered saline, among others) in order to form particles comprising the amphipathic peptides and lipids. After hydration, the peptide concentration will beneficially range from 2-4 mg/ml, among other values. Lipid concentrations will typically range from 1 to 20 mg/ml, which amount is typically dictated by the peptide concentration and the desired weight ratio of peptide to lipid.

In some embodiments, a solution of an immunogenic species (e.g., in a solvent like that used for the above lipid and amphipathic peptide stock solutions, for instance, methanol or a solvent that is miscible with methanol such as methylene chloride) is mixed with the lipid and amphipathic peptide stock solutions, dried, and rehydrated to form loaded particles comprising the amphipathic peptides and lipids in accordance with the invention. See, for example, Examples 10 and 13 below.

In some embodiments, an immunogenic species is synthesized or modified (e.g., to render it more hydrophobic or to expose a hydrophobic portion of the immunogenic species) in the presence of unloaded particles comprising the amphipathic peptides and lipids in accordance with the invention. See, for example, Examples 12, 14 and 15 below.

In other embodiments, unloaded particles comprising amphipathic peptides and lipids in accordance with the invention are contacted with the at least one immunogenic species in order to allow the association of the final particles carrying the immunogenic species. For example, a solution of the at least one hydrophobic or amphiphilic immunogenic species may be added to unloaded particles up to a point wherein the solution begins to become cloudy (indicating that the particles are no longer taking up the immunogenic species).

If the immunogenic species comprises, for example, a lipophilic anchor, the respective anchor is according to one embodiment attached to the immunogenic species before the respectively modified compound is contacted with the particles. Attachment of the anchor can be accomplished for example by chemical modification as described above. For certain applications it is advantageous to use a cleavable linker as described above. Upon mixing the at least one immunogenic species carrying a lipophilic anchor with the particles comprising the amphipathic peptides and the lipids, the lipophilic anchor of the at least one immunogenic species inserts into the lipid core of the particle (e.g., by a self-assembly process), thereby associating the at least one immunogenic species with the particles.

In another embodiment, the immunogenic species is non-covalently associated with a lipophilic anchor prior to exposure to the particles comprising the amphipathic peptides and the lipids. For example, the immunogenic species can be non-covalently associated with a capturing agent which comprises a hydrophilic head for binding the immunogenic species and a lipophilic anchor for insertion into the lipid core of the particles.

Conversely, a lipophilic anchor may be provided within the particles. For instance, the particles can be formed which comprise a capturing agent which comprises a lipophilic anchor that is inserted into the lipid core of the particle and a hydrophilic head which is subsequently available for capture of the immunogenic species (or the particles can be exposed to such a capturing agent after particle formation and prior to exposure to the immunogenic species). For example, the preceding capturing agent may be a cationic lipid which binds/captures negatively charged immunogenic species (e.g., DNA, RNA, etc.) upon exposure to the same. Analogous to the techniques described above for immunogenic species, such capturing agents may be present at the time of particle formation or may be introduced to previously formed particles.

In certain embodiments, the cationic lipid (e.g., cationic amphiphile) may be selected from the following, among others: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC) and cetyl trimethylammonium chloride (CTAC), primary amines, secondary amines, tertiary amines, including but not limited to N,N′,N′-polyoxyethylene (10)-N-tallow-1,3-diaminopropane, other quaternary amine salts, including but not limited to dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2(2-methyl-4-(1,1,3,3tetramethylbutyl)-phenoxy]-ethoxy)ethyl]-benzenemetha-naminium chloride (DEBDA), dialkyldimethylammonium salts, -[1-(2,3-dioleyloxy)-propyl]-N,N,N,trimethylammonium chloride, 1,2-diacyl-3-(trimethylammonio) propane (acyl group=dimyristoyl, dipalmitoyl, distearoyl dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (Cl₂Me₆; C₁₂Bu₆), dialkylglycetylphosphorylcholine, lysolecithin, L-a dioleoyl phosphatidylethanolamine), cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (Cl₂GluPhCnN⁺), ditetradecyl glutamate ester with pendant amino group (Cl₄GluCnN), cationic derivatives of cholesterol, including but not limited to cholesteryl-3β-oxysuccinamidoethylenetrimethylammonium salt, cholesteryl-3β-cholesteryl-3β-carboxyamidoethylenetrimethylammonium salt, cholesteryl-3β- and 3.gamma.-[N—(N′,N′-dimethylaminoetanecarbomoyl]cholesterol) (DC-Chol).

Other cationic lipids for use in the present invention include the compounds described in U.S. Patent Publications 2008/0085870 (Apr. 10, 2008) and 2008/0057080 (Mar. 6, 2008).

Conversely, the capturing agent may be an anionic lipid which binds/captures positively charged immunogenic species upon exposure to the same. In certain embodiments, the anionic lipid (e.g., anionic amphiphile) may be selected from the following, among others: phosphatidyl serine chenodeoxycholic acid sodium salt, dehydrocholic acid sodium salt, deoxycholic acid, docusate sodium salt, glycocholic acid sodium salt, glycolithocholic acid 3-sulfate disodium salt, N-lauroylsarcosine sodium salt, lithium dodecyl sulfate, 1-octanesulfonic acid sodium salt, sodium 1-decanesulfonate, sodium 1-dodecanesulfonate, sodium choleate, sodium deoxycholate, sodium dodecyl sulfate, taurochenodeoxycholic acid sodium salt, taurolithocholic acid 3-sulfate disodium salt, 1,2-dimyristoyl-sn-glycero-3-phosphate sodium salt, 1-palmitoyl-2-oleoyl-sn-glycero-3-phosphate sodium salt, 1,2-diphytanoyl-sn-glycero-3-phosphate sodium salt, dodecanoic acid sodium salt and octadecanoic acid sodium salt.

As seen from the above, in various embodiments of the invention, one or more additional species may be added subsequent to particle formation. For example, capturing agents, pharmaceuticals such as immunogenic species (e.g., antigens, immunological adjuvants, etc., with or without an associated lipophilic anchor, capturing agent, etc.), agents for adjusting tonicity and/or pH, surfactants, cryoprotective agents, and so forth, may be added subsequent to particle formation. Frequently, these additional species are added to the particles as an aqueous solution or dispersion. The resulting admixture may be lyophilized in some embodiments as previously noted.

Compositions in accordance with some embodiments of the invention can be sterile filtered (e.g., using a 200 micron filter) at any time before or after particle formation, for example, after particle formation but before the addition of any additional species, after particle formation and after the addition of any additional species, and so forth.

It has been observed that the use of charged capturing agents (e.g., cationic lipids, etc.) for particle loading with oppositely charged species (e.g., polynucleotides such as RNA, DNA, etc.) can lead to particle aggregation, for example, with controllable aggregate sizes (i.e., aggregate widths) ranging from 50 nm or less to 100 nm to 250 nm to 500 nm to 1000 nm to 2500 nm to 5000 nm to 1000 nm or more. Without wishing to be bound by theory, it is believed that this effect is due to electrostatic attraction between the charged capturing agent (which is anchored to the particles) and the oppositely charged species. In such embodiments, the charge on the outer surface of each aggregate can be modified, for example, to match the sign of the immunogenic species (where an excess of the immunogenic species is employed relative to the capturing agent) or to match the sign of the capturing agent (e.g., where an excess of the capturing agent is employed relative to the immunogenic species). Aggregate size may be modified by varying a range of parameters, for example, by varying salt concentrations within the solutions to be mixed, by varying the concentrations of the species within the solutions to be mixed, and by varying the conditions under which the solutions are mixed (e.g., rapid mixing vs. slow mixing), among other parameters.

### F. Administration

As previously indicated, compositions in accordance with the invention can be administered for the treatment of various diseases and disorders (e.g., pathogenic infections, tumors, etc.). As used herein, “treatment” refers to any of the following: (i) the prevention of a pathogen or disorder in question (e.g. cancer or a pathogenic infection, as in a traditional vaccine), (ii) the reduction or elimination of symptoms associated with a pathogen or disorder in question, and (iii) the substantial or complete elimination of a pathogen or disorder in question. Treatment may thus be effected prophylactically (prior to arrival of the pathogen or disorder in question) or therapeutically (following arrival of the same).

Compositions in accordance with the invention are typically administered to vertebrate subjects in one or more doses in pharmaceutically effective amounts. An “effective amount” of a composition in accordance with the present invention refers to a sufficient amount of the composition to treat a disease or disorder of interest. The exact amount required will vary from subject to subject, depending, for example, on the species, age, and general condition of the subject; the severity of the condition being treated; in the case of an immunological response, the capacity of the subject's immune system to synthesize antibodies, for example, and the degree of protection desired; and the mode of administration; among other factors. An appropriate effective amount in any individual case may be determined by one of ordinary skill in the art. Thus, an effective amount will typically fall in a relatively broad range that can be determined through routine trials.

Compositions in accordance with the invention can be administered parenterally, e.g., by injection (which may be needleless). The compositions can be injected subcutaneously, intradermally, intramuscularly, intravenously, intraarterially, or intraperitoneally, for example. Other modes of administration include nasal, mucosal, intraoccular, rectal, vaginal, oral and pulmonary administration, and transdermal or transcutaneous applications.

In some embodiments, the compositions of the present invention can be used for site-specific targeted delivery. For example, intravenous administration of the compositions can be used for targeting the lung, liver, spleen, blood circulation, or bone marrow.

Treatment may be conducted according to a single dose schedule or a multiple dose schedule. A multiple dose schedule is one in which a primary course of administration may be given, for example, with 1-10 separate doses, followed by other doses given at subsequent time intervals, chosen to maintain and/or reinforce the therapeutic response, for example at 1-4 months for a second dose, and if needed, a subsequent dose(s) after several months. The dosage regimen will also be determined, at least in part, by the need of the subject and the judgment of the practitioner.

Furthermore, if prevention of disease is desired, the compositions are generally administered prior to the arrival of the primary occurrence of the infection or disorder of interest. If other forms of treatment are desired, e.g., the reduction or elimination of symptoms or recurrences, the compositions are generally administered subsequent to the arrival of the primary occurrence of the infection or disorder of interest.

The following Examples are intended to further illustrate the invention and are not to be construed as being limitations thereon.

### EXAMPLES

### Example 1

### Materials Used

POPC is from Chemi (Basalmo, Italy), DOPC, DMPC and DPPC are from Avanti Polar Lipids (Alabaster, Ala.), Peptides, SEQ ID NO: 1, SEQ ID NO: 3, SEQ ID NO: 4 and SEQ ID NO: 5, are from American peptide company Inc. (Sunnyvale, Calif.), methanol HPLC grade is from Acros (Pittsburgh, Pa.), DiI is from Invitrogen (Carlsbad, Calif.), and Human HDL, LDL and VLDL are from Millipore Corp. (Billerica, Mass.).

### Example 2

### Preparation of the Particles

Stock solutions of peptide and lipid are made in methanol at a concentration of 10 mg/ml. Necessary aliquots of the stocks are transferred to glass vials to obtain peptide to lipid molar ratios ranging from 1:0.877 to 1:7 (weight ratios of 4:1 to 1:2). The mixture is vortexed and methanol is evaporated on a rotovap to obtain a clear lipid-peptide film. The particles are obtained by hydrating the film with sterile filtered normal saline at a peptide concentration of 2 mg/ml.

### Example 3

### Measurement of the Particle Size by Dynamic Light Scattering

The particles formed by hydration are characterized for particle size by dynamic light scattering on a Malvern Zetasizer Nano-ZS (Malvern Instruments, Milford Mass.) at a back scattering angle of 173°. Undiluted particles are used for measurements.

The peptide of SEQ ID NO: 1 is used to form particles at peptide to lipid molar ratios of 1:1.75, 1:3 and 1:7 and the size is characterized by dynamic light scattering, number average reported in nm followed by polydispersity index in parenthesis: (a) 5.80 nm (0.1), 15.80 nm (0.15) and 19.92 nm (0.27) respectively with lipid POPC, (b) 8.28 nm (0.4), 11.26 nm (0.16) and 1.72×10⁴ nm (1.0) respectively with lipid DOPC, (c) 10.04 nm (1.0), 17.34 nm (0.76) and 46.14 (0.67) respectively with lipid DPPC, and (d) 5.24 nm (0.15), 5.51 nm (0.16) and 7.12 nm (0.05) respectively with lipid DMPC.

Other amphipathic peptides (SEQ ID NOs: 2, 3 and 4) are also evaluated for particle formation with lipid POPC. The particles are formed at peptide to lipid molar ratios of 1:1.75, 1:3 and 1:7, and characterized by dynamic light scattering for particle size, number average reported in nm followed by polydispersity index in parenthesis: (a) 5.21 nm (0.7), 5.70 nm (0.68) and 27.82 nm (0.63) respectively with peptide SEQ ID NO: 2, (b) 4.67 nm (0.1), 8.18 nm (0.13) and 5.95×10⁴ nm (1.0) respectively with peptide SEQ ID NO: 3, and (c) 5.24 nm (0.21), 6.26 nm (0.23) and 8.74 nm (0.57) respectively with peptide SEQ ID NO: 4.

### Example 4

### Size Exclusion Chromatography

The particles are sized on an Akta explorer 900 (Amersham Biosciences) using superpose-6 column (GE Health Care Life Sciences). The particles are eluted with 50 mM Sodium phosphate with 150 mM sodium chloride at a flow rate of 0.5 ml/min for about 40 ml volume. 0.5 ml fractions of the eluent are collected into a 96-well plate (with 8 rows from A to H and 12 columns 1 to 12) in a row fashion starting from A₁ to A₁₂ followed by row B to H. Data is collected at 215 nm, 254 nm and 280 nm. A mixture of low and high molecular weight gel filtration markers of known stokes diameter are run under similar conditions. The size of the particles is determined by comparing the elution volumes of the samples with that of the standards.

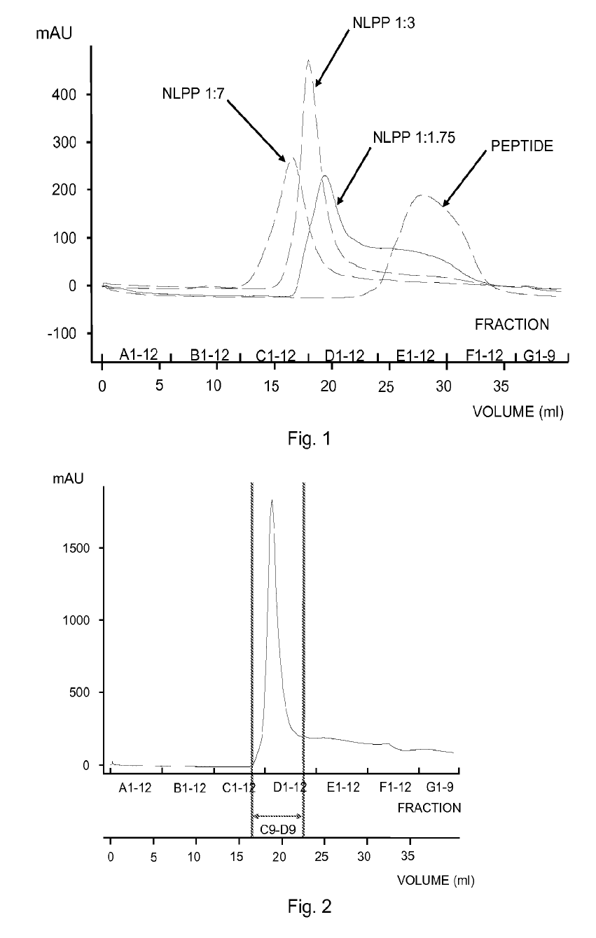

The particles from peptide SEQ ID NO: 1 and lipid POPC at peptide to lipid molar ratios of 1:1.75, 1:3 and 1:7 are prepared and are characterized by size exclusion chromatography (see FIG. 1). Elution peak fraction (elution volume in ml) and Stokes diameter (in nm) of the particles are as follows: (a) at a molar ratio of 1:1.75 the particles are eluted at D₃ (19.39 ml) and had a stokes diameter of 2.92 nm, (b) at a molar ratio of 1:3 the particles are eluted at C₁₋₂-D₁ (18.14 ml) and had a stokes diameter of 4.70 nm, and (c) at a molar ratio of 1:7 the particles are eluted at C₉ (16.39 ml) and had a stokes diameter of 7.20 nm.

### Example 5

### Concentration by Tangential Flow Filtration (TFF)

Following size exclusion the particles are concentrated using MicroKros hollow fibers (Spectrum Labs). A 50 KD cut off Microkros module is used for this purpose. The luerlok sample ports are connected through a peristalitic pump for continuous flow of the sample through the system. The designated luerlok is connected to the filtrate or waste which is collected. All the connections are made with tubing of smallest diameter in order to reduce the void volumes of the whole system. The whole concentration process is stopped when the volume of the sample are equal to or lower than the void volume and are indicated by the introduction of air bubbles into the system. The MicroKros filter is pre-wetted with normal saline before use.

Particles made from peptide Seq ID No.1 and lipid POPC at a molar ratio of 1:1.75 are used. About 200 μl of the particles at a peptide concentration of 8 mg/ml are injected on to a Superose column to perform size exclusion (see FIG. 2). The peak fractions C₉-D₉ are combined to give 6.5 ml and are concentrated to 2 ml by TFF. The pooled fractions are characterized by dynamic light scattering for particle size, number average reported in nm followed by polydispersity index in parenthesis 5.6 nm (0.242), after concentration these parameters for the particles are found to be at 6.69 nm (0.424) and comparable to these parameters for the unprocessed particles (before size exclusion chromatography) at 5.80 nm (0.1). These data demonstrate that the particles and specifically their size do not change when they are concentrated. The particles are thus remarkably stable and do not form substantial amounts of aggregates or other artificial products under the preceding conditions. It is also shown that the particles can be sterile filtered and still remain stable.

### Example 6

### SEC Fraction Analysis for Peptide and Lipid Content

The peptide content of the pooled/concentrated fractions is analyzed by UV absorbance at 215 nm. The lipid content is estimated using Phospholipid C reagent (Wako Diagnostics, Japan), a colorimetric enzymatic assay for determination of phospholipids. The absorbance of the chromogen is measured at 600 nm.

About 200 μl of particles made from peptide SEQ ID NO: 1 and lipid POPC at a molar ratio of 1:1.75 are injected on to a superpose column at a peptide concentration of 8 mg/ml (see FIG. 2). Following size exclusion the peak fractions C₉-D₉ are combined and are concentrated by TFF. The pooled fractions after size exclusion are estimated to have 1.27 mg of the peptide and 0.35 mg of lipid. After TFF the retentate contains 0.92 mg of peptide and 0.35 mg of lipid, and 0.04 mg of peptide is found in the filtrate waste.

### Example 7

### Characterization of Particles by NMR

Nuclear overhouser effect spectroscopy (NOESY), 2-D NMR is used to study the peptide-lipid interactions and particles formed are evaluated by 1-D NMR. The peptide-lipid films are prepared as described and hydrated using 5 mM potassium phosphate (KH₂PO₄) buffer made in 90% v/v H₂O and 10% v/v D₂O at pH 6.23, 37° C. The particles formed from peptide Seq ID No.1 and lipid POPC (molar ratio 1:1.75) at a concentration of 2 mg/ml are used to collect data on Bruker-Biospin NMR at 600 MHz.

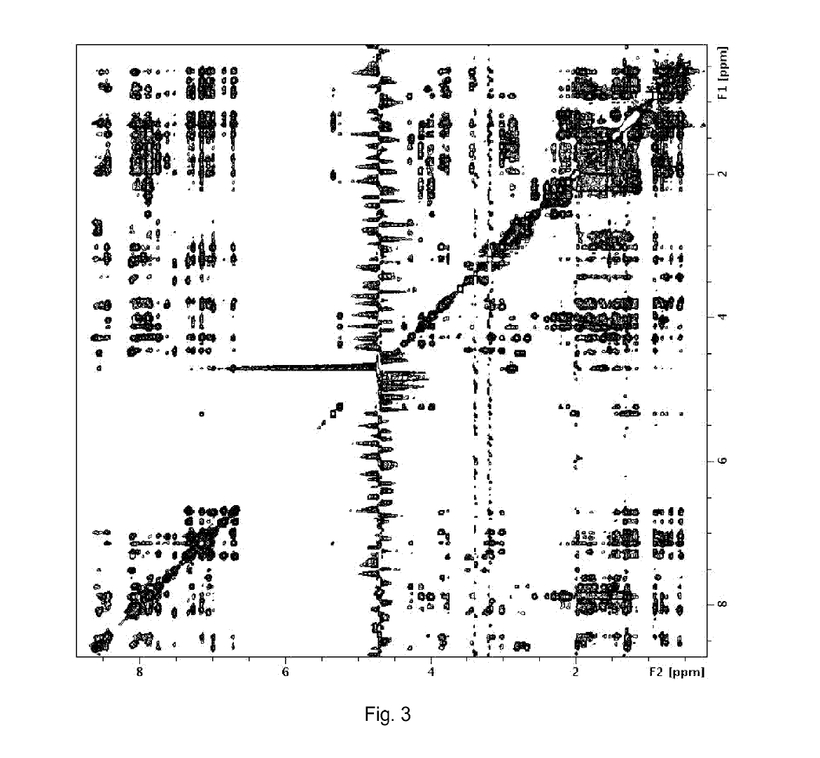

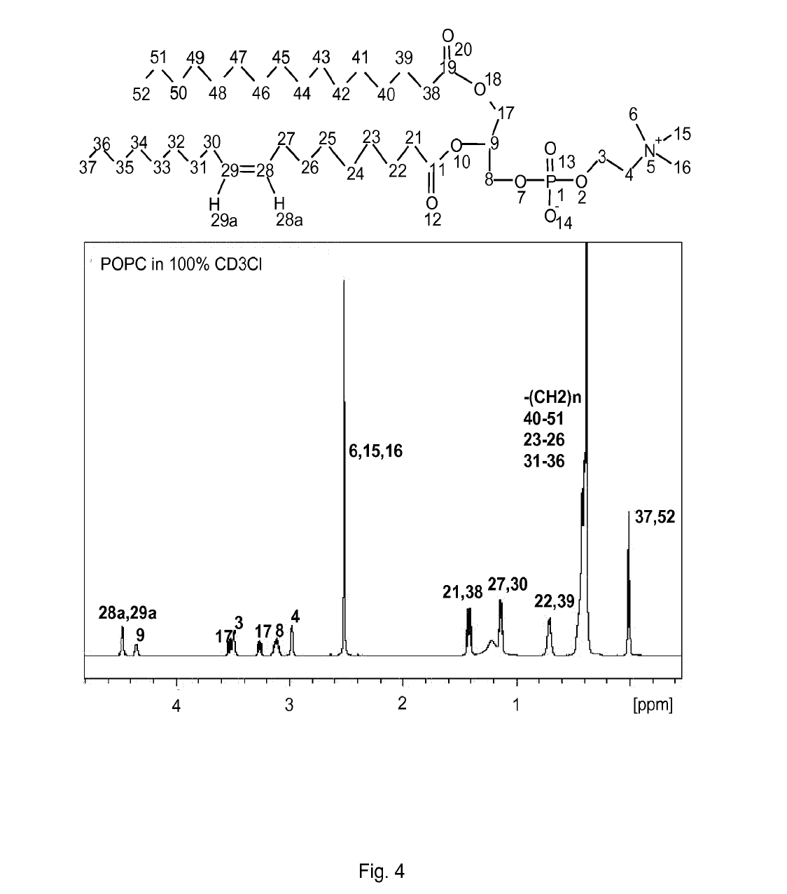

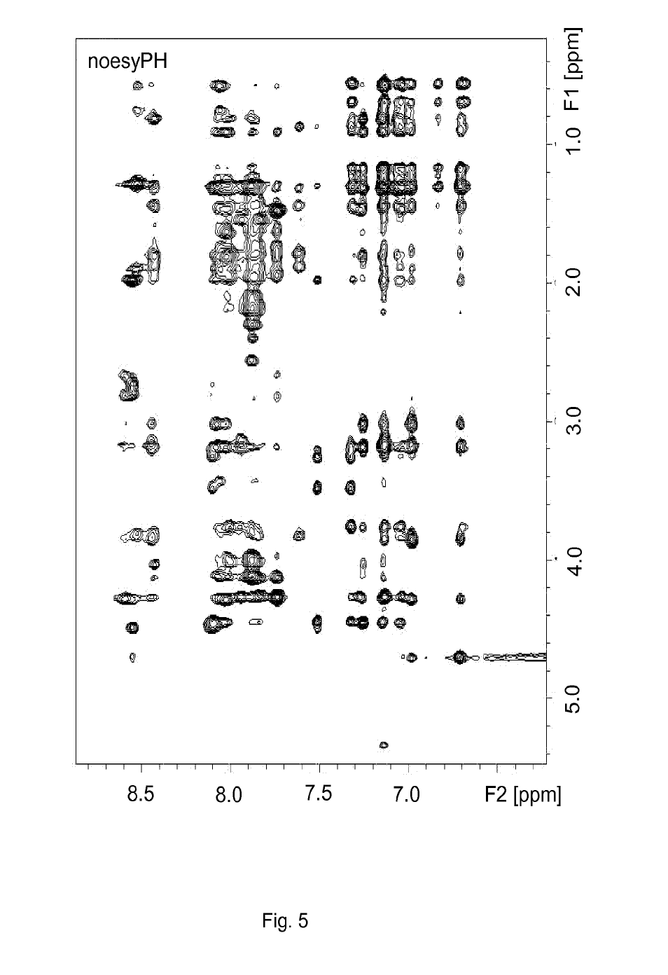

NOESY uses dipolar interaction of spins to correlate protons, this correlation depends on the distance between protons and a NOE signal is observed only when the distance is less than 5 \acute{Å}. The spectra of particles has NH—NH NOE signals which indicate the interactions of α-proton to α-proton and confirm the helical structure of the peptide (see FIG. 3). In the 2-D NMR of particles, the x-axis dimension from 6-9 ppm shows protons from aromatic ring and backbone of the peptide (N—H), and the Y-axis dimension from 0-5 ppm shows signal from protons of lipid and side chains of the peptide. The proton assignment of aromatic amino acids tyrosine (Y—6.99, 7.22 ppm), and phenylalanine (F—7.31 ppm), and lipid the double bond linked protons at 4.5 ppm resolved from the rest are known from 1-D NMR (see FIG. 4). The NOE signals at the intersection of 6.99, 7.22 and 7.31 ppm (on x-dimension) with 4.5 ppm (on Y-dimension) indicate the interactions between the protons of aromatic amino acids with double bond linked protons of the lipid (see FIG. 5).

### Example 8

### In Vitro Stability of Particles by Size Exclusion

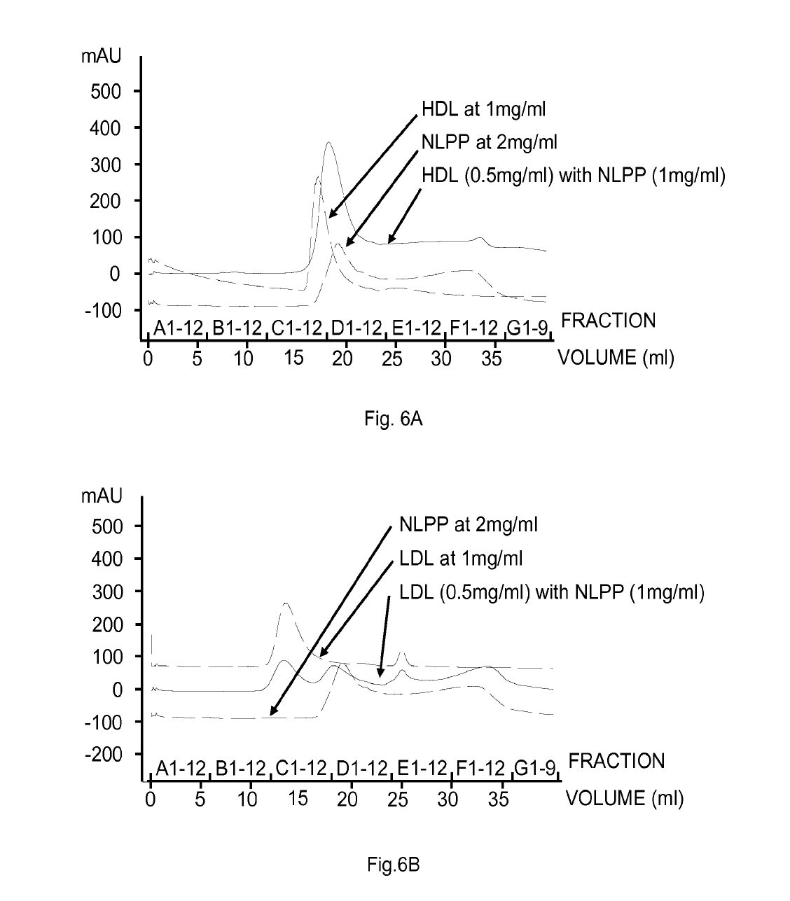

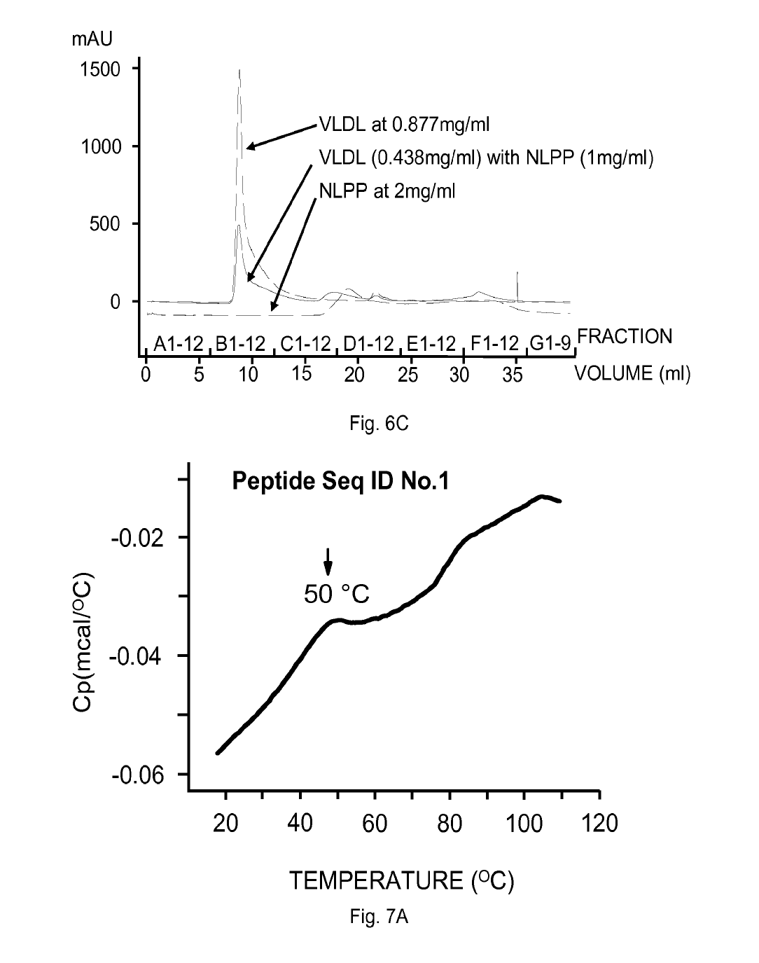

In order to study the stability of particles, the particles are co-incubated in presence of human lipoproteins (HDL, LDL and VLDL) and are characterized by size exclusion chromatography. The particles with peptide to lipid molar ratio of 1:1.75 are used. The particles with a final peptide concentration of 1 mg/ml are incubated with individual lipoproteins at 0.5 mg/ml, and injected on to the size exclusion column. The particles are found to co-elute along with HDL but are seen to exist as a distinct peak when injected with LDL and VLDL. In both cases, a slight shift in the particle peak is observed (see FIGS. 6A-6C).

### Example 9

### In Vitro Stability of Particles by Differential Scanning Calorimetry

Differential scanning calorimetry is used to study the unfolding events associated with the peptide and particles. This technique is used to measure the amount of heat required to increase the temperature of the sample and reference, resulting in peaks at phase transition temperatures at which more heat is required by the samples to be maintained at the same temperature as the reference. In case of proteins the melting temperatures are determined at which half of the protein exists in an unfolded state.

The peptide of SEQ ID NO: 1 is used to form particles at peptide to lipid (POPC) molar ratios of 1:1.75, 1:3 and 1:7, the particles with peptide at concentration of 1.11 mg/ml and lipid at 0.55 mg/ml are used. The peptide alone and lipid alone are used as controls and the samples are scanned from 20° C. to 130° C.

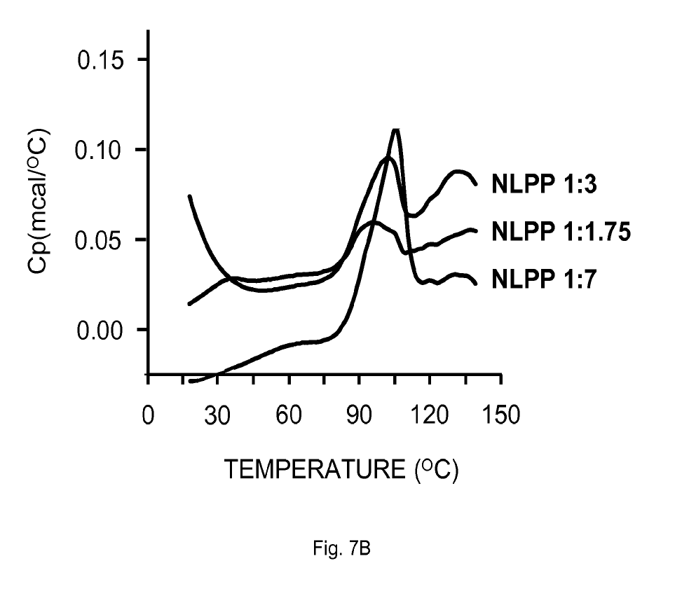

FIGS. 7A-7B show differential scanning calorimetry of peptide and particles. The plots show melting curves of (a) peptide SEQ ID NO: 1 at 1.11 mg/ml and (b) particles made from peptide SEQ ID NO: 1 and lipid POPC at peptide to lipid molar ratios of 1:1.75, 1:3, and 1:7 at peptide concentrations of 1.11 mg/ml in particles. All samples of peptide and particles were made in normal saline. The DSC curves obtained show a phase transition of peptide alone at 50° C. and, for particles with peptide to lipid molar ratio at 1:1.75 a phase a transition at 105° C. is observed, for particles with peptide to lipid molar ratio at 1:3 and 1:7 a phase transition of 93° C. is observed. Thus, these figures show that the particles have a rather high melting point of more than 90° C. This stability is desirable for an industrial large scale production and for the handling of the particles.

### Example 10

### Incorporation of Lipopeptide into NLPP

Lipids (POPC, DOPC, DMPC and DPPC) were obtained from Sigma (Sigma-Aldrich, Italy) and methanol HPLC grade was obtained from Sigma (Sigma-Aldrich, Italy). The peptide corresponds to SEQ ID NO: 1. The lipopeptide palmitoyl-Cys(2[R],3-dilauroyloxy-propyl)-Abu-D-Glu-NH₂ was synthesized and provided as the carboxylic acid (waxy solid):

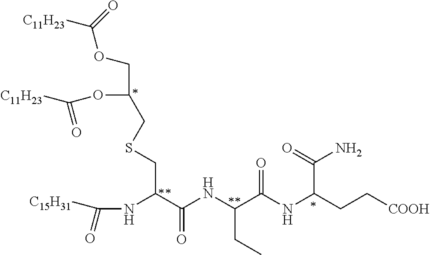

where the chiral centers labeled * are in the R configuration, and ones labeled ** are in the S configuration. This lipopeptide is referred to herein as Lipopeptide 1 (Lipo 1) and its synthesis is described in Example 16 of U.S. Pat. No. 4,666,886 to Baschang et al. which is incorporated herein by reference.

Stock solutions of peptide and lipid were made in methanol at a concentration of 10 mg/ml. Lipopeptide stock was made in methanol at a concentration of 3 mg/mL. Necessary aliquots of the stocks were transferred to glass vials to obtain the desired peptide:lipid:lipopeptide weight ratio. The mixture was vortexed and methanol was evaporated on a rotovap to obtain a clear lipid-peptide film. Particles were obtained by hydrating the film with sterile filtered normal saline added to achieve a lipopeptide concentration of 1 mg/ml, corresponding to ca. 10 mM.

The particles formed by hydration were characterized for particle size by dynamic light scattering on Malvern zetasizer Nano-ZS (Malvern Instruments, Milford Mass.) at a back scattering angle of 173°. Undiluted particles were used for measurements. Number average size is reported in nm, followed by polydispersity index in parenthesis, for the following: (a) peptide:lipid:lipopeptide weight ratio 1:0:1 size 16.9 nm (0.9) with lipid POPC; (b) peptide:lipid:lipopeptide weight ratio 1:0.5:1 size 11.40 nm (0.6) with lipid POPC, (c) peptide:lipid:lipopeptide weight ratio 1:0.75:1 size 63.8 nm (1) with lipid POPC; (d) peptide:lipid:lipopeptide weight ratio 1:0.5:0.5 size 89 nm (0.6) with lipid DMPC; (e) peptide:lipid:lipopeptide weight ratio 1:0.5:0.5 size 600 nm (0.2) with lipid DOPC; and (f) peptide:lipid:lipopeptide weight ratio 1:0.5:0.5 size 557 nm (0.4) with lipid DPPC.

### Example 11

### In Vitro Activity on TLR2 Expressing Cells

HEK293 cells stably transfected with a reporter vector in which the luciferase gene is under the control of an NFkB dependent promoter (HEK293-NF-κBLuc cells) were obtained as follows: cDNA for the Firefly luciferase open reading frame (ORF) was amplified by PCR and subcloned in the pNFkB reporter vector (Cell and Molecular Technologies Inc.) to obtain the pNFkB-luc reporter vector. HEK293 cells were co-transfected with pNFkB-luc reporter vector and the pTK-puro expression vector and cultured in the presence of the selection antibiotic puromycin (5 ug/ml). Individual resistant clones were selected, expanded and tested for luciferase expression/activity upon stimulation with a positive stimulus. Clone LP58 was selected for further studies.

These cells (clone LP58) were transfected using Lipofectamine 2000 following manufacturer's instructions (Invitrogen, Carlsbad, Calif.) with pcDNA3.1-Hydro-FLAG-hTLR2 plasmid encoding for human TLR2 containing a FLAG epitope at the NH₂ terminus and a hygromycin resistance gene for selection. Transfected cells were cultured in the presence of selection antibiotic hydromycin (250 μg/ml) and individual resistant clones were selected, expanded and tested for expression of luciferase upon stimulation with the TLR2 specific agonist PAM₃CSK₄ (Invitrogen, Carlsbad, Calif.). Clone 6 was selected for further studies. HEK293-FLAG-TLR2-NF-κB-Luc cells

Cells (clone 6) were cultured in Dulbecco's Modified Eagle Medium (DMEM) containing 4500 mg/l glucose, supplemented with 10% heat-inactivated FCS (HyClone), 100 U/ml penicillin, 100 μg/ml streptomycin, 2 mM glutamine, 5 μg/ml puromycin and 250 μg/ml hygromycin. For the luciferase assay, HEK293 transfectants were seeded into microclear 96-well plates in 90 μl of complete medium (25×10³ cells/well) in the absence of selection antibiotics. After overnight incubation, cells were stimulated in duplicates with the different stimuli (10 μl/well) for 6 hours. Then, medium was discarded and cells were lysed with 20 μl Passive Lysis Buffer (Promega) for 20 min at room temperature. Luciferase levels were measured by addition of 100 μl/well Luciferase Assay Substrate (Promega) using the LMax II³⁸⁴ microplate reader (Molecular Devices). Raw light units (RLU) from each sample (average of 2) were divided by the RLU of the control sample (PBS) and expressed as Fold increase (FI). PAM₃CSK₄ dissolved in PBS was used as positive control for activation of TLR2 transfected cells. PAM₃CSK₄ is (S)-[2,3-Bis(palmitoyloxy)-(2-RS)-propyl]-N-palmitoyl-(R)-Cys-(S)-Ser-(S)-Lys₄-OH. The form used is the trihydrochloride form, which is available form Invivogen. It is a synthetic tripalmitoylated lipopeptide that mimics the acylated amino terminus of bacterial lipoproteins, and is a selective agonist of human and mouse TLR2. See J. Metzger, et al.; Int. J. Pept. Protein Res. 37, 46 (1991) and A. O. Aliprantis et al., Science 285(5428): 736-739 (1999).

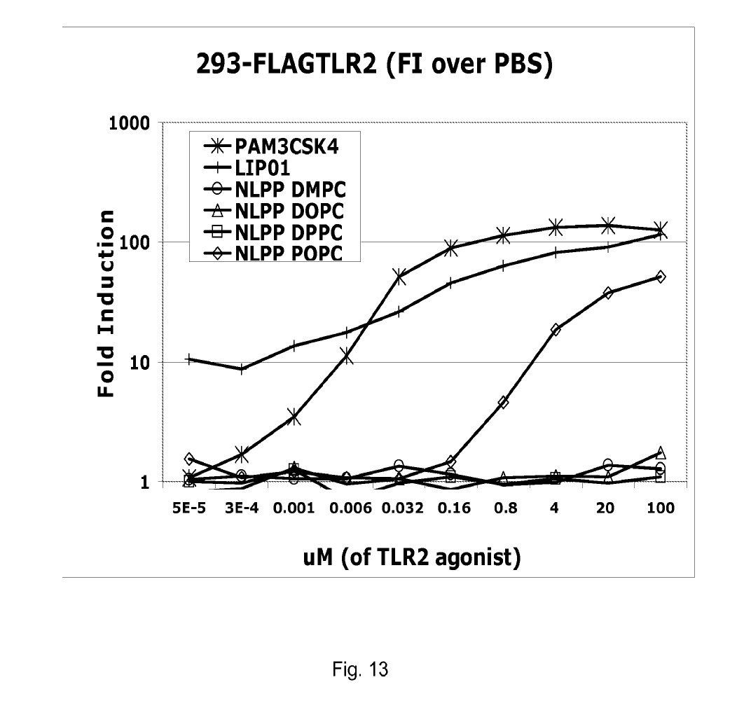

The different types of empty NLPPs (without the lipopeptide) were tested for their ability to stimulate TLR2 transfectants and compared to stimulation by PAM₃CSK₄ and sonicated lipopeptide as shown in FIG. 13. Stimulation could be observed only with High concentration of NLPP containing POPC.

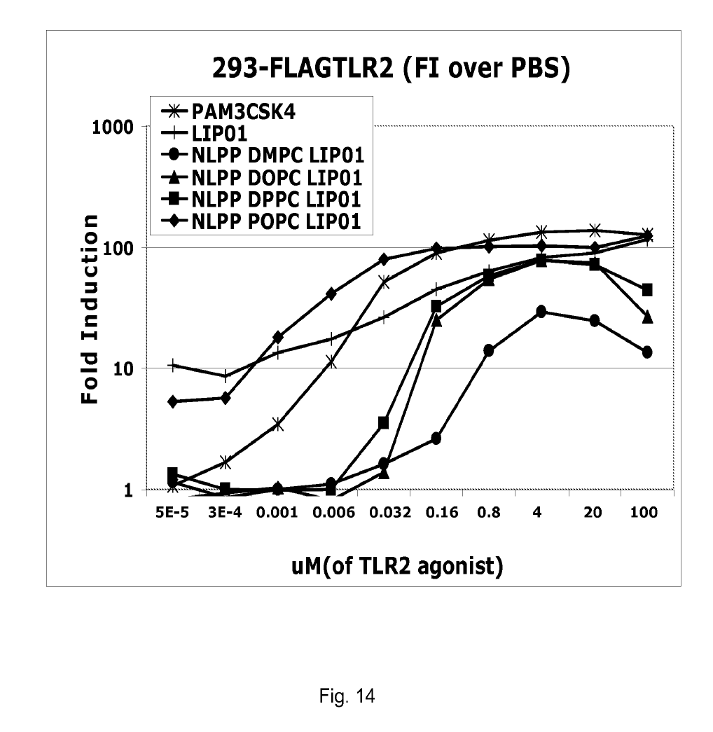

Then the response of TLR2 cells to different NLPPs containing the lipopeptide was evaluated. See FIG. 14. A dose dependent response was observed for all forms of NLPP containing the lipopeptide. However, the best response was induced by NLPP containing POPC, which showed higher activity compared to both sonicated lipopeptide and the TLR2 benchmark activator PAM₃CSK₄

### Example 12

### In Vitro Activity on Human Peripheral Blood Mononuclear Cells (PMBC) and Mouse Splenocytes

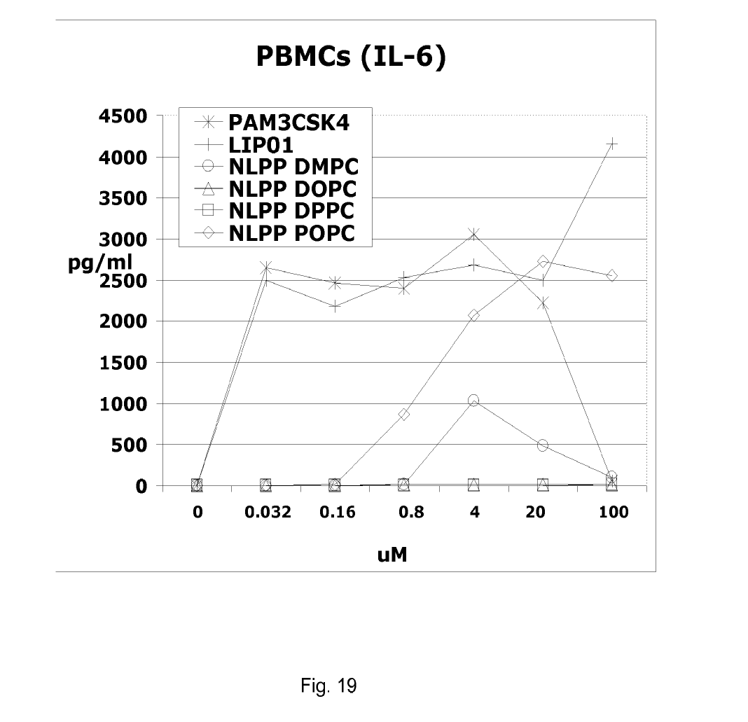

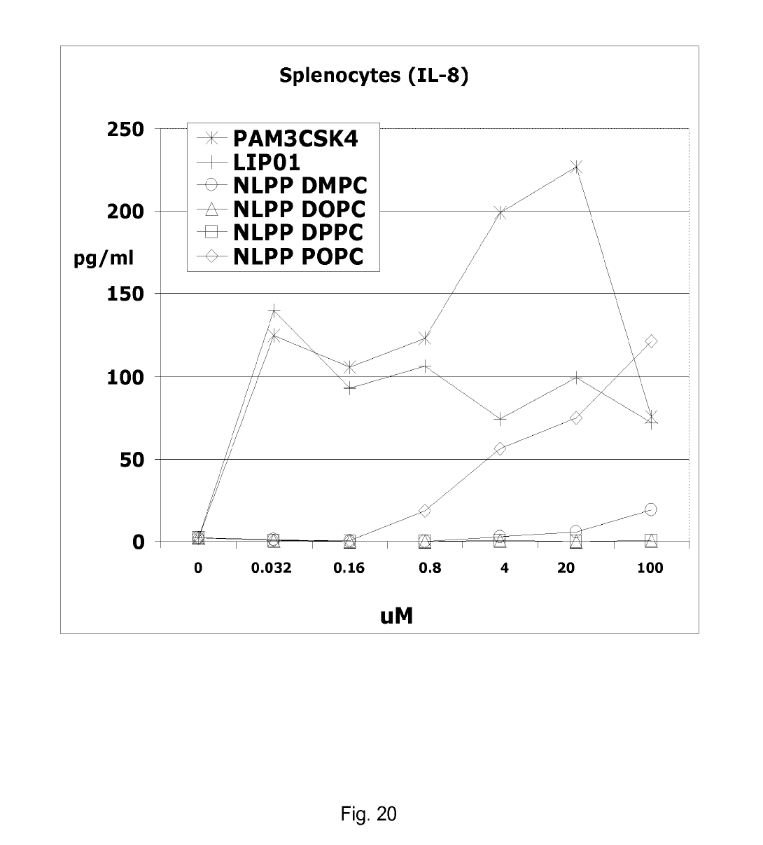

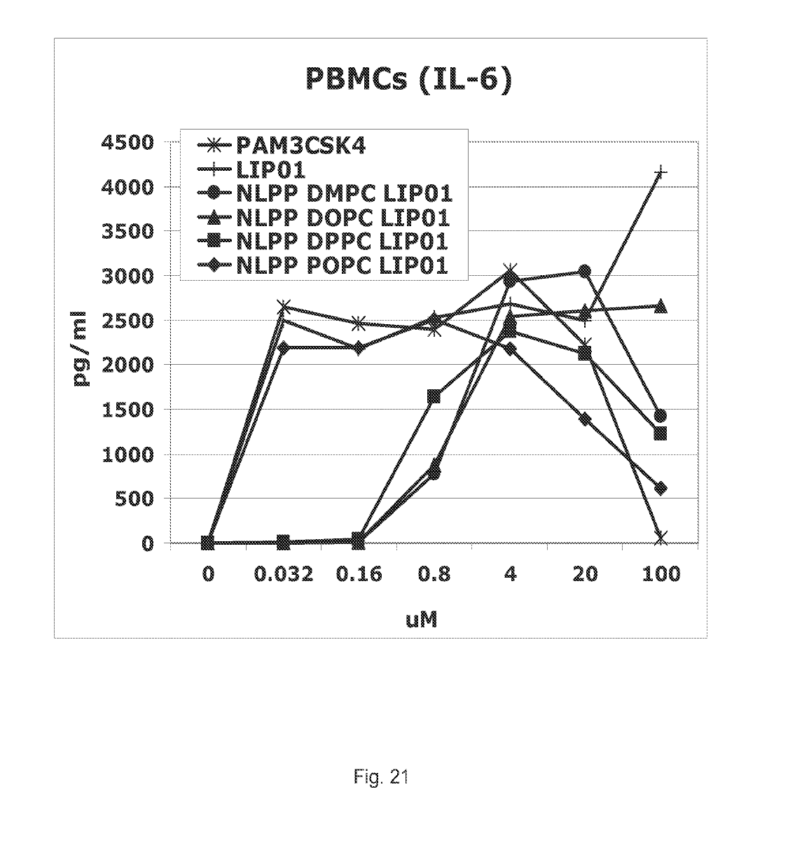

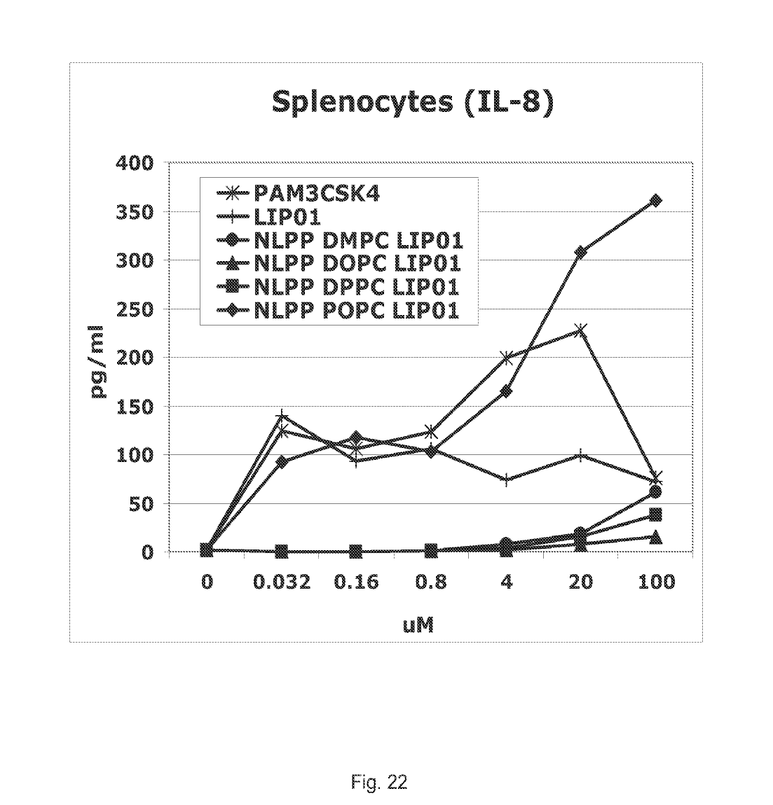

Complete medium for human PBMC is as follows: RMPI1640 medium supplemented with 10% heat-inactivated FCS (HyClone), 100 U/ml penicillin, 100 μg/ml streptomycin, 2 mM glutamine. Complete medium for mouse splenocytes is as follows: RMPI1640 medium supplemented with 2.5% heat-inactivated FCS (HyClone), 100 U/ml penicillin, 100 μg/ml streptomycin, 2 mM glutamine, with the addition of 2-mercaptoethanol 50 uM. Human PBMC were purified from blood of healthy donors using Ficoll gradient. Mouse splenocytes were purified from spleen of Balb/c mice as follow: spleens were smashed and cells filtered through a cell strainer; cells were washed once with complete medium, resuspended in the LCK buffer (NH₄Cl 155 mM, KHCO₃ 1 mM, EDTA-2Na 0.1 mM, pH7.4) for 2 minutes to lyse red blood cells, washed and resuspended in complete medium. Both type of primary cells were seeded into 96-well flat bottom plates (1×10⁵/well) in 180 μl medium and stimulated in duplicates (20 μl/well). After 20 hours, supernatants were collected and a multiplex measurement of secreted cytokines (IL-1β, IL6, IL-8, IL-10, IL-12p70, IFNγ, TNFα) was performed using a Mesoscale kit following manufacturer's instructions. PAM₃CSK₄ dissolved in PBS was used as positive control for activation of the primary human and mouse cells. The different type of empty NLPPs (without Lipo 1) were tested for their ability to stimulate human PBMC (FIG. 19), and mouse splenocytes (FIG. 20), and compared it to stimulation by PAM₃CSK₄ and sonicated Lipo 1. Stimulation could be observed only with high concentration of NLPP containing POPC in both PBMC (IL-6 production shown in FIG. 19) and mouse splenocytes (IL-8 production shown in FIG. 20). Then the response to different NLPPs containing Lipo 1 was evaluated on human PBMC (FIG. 21) or mouse splenocytes (FIG. 22). A dose dependent response was observed in IL-6 production by human PBMC for all forms of NLPP containing the lipopeptide Lipo 1, although the best response was induced by NLPP-POPC (FIG. 21). NLPP-POPC containing the lipopeptide Lipo 1 was also able to induce IL-8 release from mouse splenocytes while no effect on these cells was observed for all the other type of NLPPs containing the lipopeptide Lipo 1 (FIG. 22).

### Example 13

### Incorporation of RSV F into NLPP

1:4 ratio peptide:DMPC NLPP were prepared as described above, i.e., 1:4 peptide:DMPC weight ratio, hydrated in PBS to a concentration of 2 mg/mL peptide, equivalent to 10 mg/mL of bulk particles (see Example 4), and the sample diluted to either 500 micrograms/ml or 200 micrograms/ml in water. The diluted samples were loaded onto glow-discharged carbon coated grids (EM Science), the grids were washed with water, and stained with 0.75% urinal formate stain (EM Science). Images were recorded on a JOEL JEM-1200EX electron microscope with a voltage of 80 kV and magnification of approximately 30,000×.

EM analysis of NLPP containing 1:4 Peptide:DMCP ratio was conducted. NLPP loaded at 500 mcgs/ml showed NLPPs stacking in a linear fashion. When the NLPP sample was diluted to 200 mcgs/ml, the disks were more dispersed and of approximately 15-25 nm in size.

Transmembrane region-deleted, 6-HIS tag fused RSV F protein with furin cleavage site mutated (Delp23 Furdel) was expressed in HiFive insect cells in Express Media (Invitrogen) and purified using HiTrap chelating column and Superdex P200 16/60 column (GE Healthcare). More particularly, the HIS-tagged RSV F construct, lacking transmembrane domain and harboring mutations to its native furin cleavage sites (Delp23 Furdel) were cloned into pFastBac plasmids for baculovirus formation (Invitrogen).

RSV F Delp23 Furdel with a hexa histidine tag has the following sequence:

(SEQ ID NO: 8)
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATNNRARQ-----------------------
QQQRFLGFLLGVGSAIASGVAVSKVLHLEGEVNKIKSALLSTNKAVVSLS
NGVSVLTSKVLDLKNYIDKQLLPIVNKQSCSISNIETVIEFQQKNNRLLE
ITREFSVNAGVTTPVSTYMLTNSELLSLINDMPITNDQKKLMSNNVQIVR
QQSYSIMSIIKEEVLAYVVQLPLYGVIDTPCWKLHTSPLCTTNTKEGSNI
CLTRTDRGWYCDNAGSVSFFPQAETCKVQSNRVFCDTMNSLTLPSEVNLC
NVDIFNPKYDCKIMTSKTDVSSSVITSLGAIVSCYGKTKCTASNKNRGII
KTFSNGCDYVSNKGVDTVSVGNTLYYVNKQEGKSLYVKGEPIINFYDPLV
FPSDEFDASISQVNEKINQSLAFIRKSDELLHNVNAGKSTTNGGSAGSGH
HHHHH
(the symbol “-” indicates that the amino acid at
this position is deleted).

The baculovirus stock was amplified to high titer using Sf9 cells (Invitrogen). Protein was expressed in HiFive cells (Invitrogen) approximately 10 mls of passage number 3 baculovirus stock were added to every liter of HiFive cells at 2×10⁶/ml. Expression was allowed to go for ˜72 hours. Cells were harvested, after taking an aliquot of cell/media suspension for SDS-PAGE analysis, by pelleting the cells from the media by centrifuging the cells at 3000 r.p.m. for ˜30 mins Copper (II) sulfate was added to the media to a final concentration of 500 micromolar and 1 liter of media with copper was added to ˜15 mls of chelating IMAC resin (BioRad Profanity). Protein-bound resin was separated from flow-through using a gravity column. Resin was washed with at least 10× resin volume of equilibration buffer (25 mM Tris pH 7.5, 300 mM NaCl) and protein was eluted with at least 10× resin volume of elution buffer (25 mM Tris pH 7.5, 300 mM NaCl, 250 mM Imidazole). Eluted sample was spiked with EDTA-free complete protease inhibitor (Pierce) and EDTA to a final concentration of 1 mM. Elution solution was dialyzed at least twice at 4° C. against 16× volume equilibration buffer. Eluted sample was loaded onto one or two HiTrap Chelating columns preloaded with Ni++ (a single 5 ml column was typically sufficient for 10 liters of expression) and protein was eluted off using fast protein liquid chromatography (FPLC) capable of delivering a gradient of elution buffer with the following gradient profile (2 ml/min flow rate): (a) 0 to 5% Elution buffer over 60 mls, (b) 5 to 40% Elution buffer over 120 mls and (c) 40 to 100% Elution buffer over 60 mls. Fractions containing RSV F protein were identified using SDS-PAGE analysis using coomassie and/or western staining (typically, RSV F elutes off ˜170 mls into the gradient). The material was concentrated to approximately 0.5-1 mg/ml and EDTA added to 1 mM final concentration. Using an FPLC, collecting 1 ml fractions, with a 16/60 Superdex P200 column (GE Healthcare) with equilibration buffer as the mobile phase, the RSV F material (retention volume approximately 75 mls) was resolved from the insect protein contaminates (retention volume approximately 60 mls). Fractions containing highly pure RSV F Delp23 Furdel material were identified using SDS-PAGE with Coomassie staining and relevant fractions were pooled and concentrated to a final protein concentration of approximately 1 mg/ml.

The Delp23 Furdel mutations have arginine residues remaining in the furin cleavage site which are susceptible to trypsin cleavage. The result is the engineered F0 species is converted to the native viral F1/F2 species with the fusion peptide exposed. EM analysis has confirmed this cleavage causes the RSV F postfusion constructs to form rosettes by virtue of their fusion peptides as has been observed for related fusion proteins.

Lyophilized Trypsin from Bovine Plasma (Sigma) was suspended and diluted to a 0.1 mg/ml concentration in 25 mM Tris pH 7.5, 300 mM NaCl. A solution of 1 mg/ml RSV F Delp23 Furdel was treated with equal volume 0.1 mg/ml trypsin solution (ratio 0.1:1 trypsin:RSV F) for 1 hour at 37 C. EM analysis of RSV F Delp23 Furdel construct before and after trypsin cleavage shows a change from a primarily crutch-shaped trimer into rosettes, as expected due to exposure of the fusion peptide.

To produce RSV F protein incorporated into NLPPs, the above reaction is repeated, but the preformed NLPP sample is added to the to RSV F Delp23 Furdel protein at a mass ratio of 0.1:1 NLPP:RSV F. Cleaved RSV F samples were diluted to approximately 50 micrograms/ml in dilution buffer and loaded onto glow-discharged carbon-coated grids and stained with urinal formate as was done with NLPP samples (above).

When RSV F Delp23 Furdel was trypsin digested in the presence of preformed NLPPs, some RSV F molecules incorporate, by virtue of their newly exposed fusion peptide, into the lipid face of the NLPP. Various species of “rosettes” were observed including (a) species that appear circular, as those observed for RSV F rosettes cleaved in the absence of NLPP and (b) species that appear as several crutch-shaped molecules, consistent with RSV F, associated with the two faces of an elongated disc. Measurement of a crutch associated with a disk was 148 angstroms, consistent with the predicted length of the RSV F postfusion trimer. Measurement of the center disk in the RSV F rosette was 16.9 nm, consistent within the range of observed sizes of the NLPP disk alone.

### Example 14

### Preparation of Particles with Small Molecule Immune Potentiator (SMIP) Compounds

Stock solutions of peptide (SEQ ID NO: 1, American Peptide Company, Sunnyvale, Calif.); 1,2-dimyristoyl-sn-glycero-3-phosphocholine lipid (DMPC, Genzyme Pharmaceuticals, Cambridge, Mass.), 1-Palmitoyl-2-oleoyl-sn-glycero-3-phosphocholine (POPC, Avanti Polar Lipids, Alabaster, Ala.), and 1,2-dioleoyl-sn-glycero-3-phosphocholine (DOPC, Avanti Polar Lipids) were prepared in methanol at concentrations of 10 mg/mL for peptide and 20 mg/mL for lipids. The peptide and lipid stock solutions were combined in glass scintillation vials at: peptide:DMPC weight ratios in the range of 1:0.5 to 1:5; peptide:POPC weight ratios of 1:0.5, 1:1, and 1:2; and peptide:DOPC weight ratios of 1:0.5, 1:1, and 1:2. Thin films of lipid and peptide were cast by solvent rotary evaporation, and subsequently hydrated in a volume of phosphate buffered saline to reach a final peptide concentration of 2-4 mg/mL.

Particle sizes were determined by dynamic light scattering using a Malvern Nano-ZS instrument (Malvern Instruments, Milford Mass.). The z-average diameter, as determined by scattering intensity, and polydispersity (in parenthesis) for the particles at peptide:DMPC weight ratios of 1:0.5, 1:1, 1:1.5, 1:2, 1:3, and 1:4 were: 7.556 nm (0.251), 7.953 nm (0.098), 8.839 nm (0.067), 10.15 nm (0.089), 13.84 nm (0.142), and 33.23 nm (0.177), respectively.

Particle sizes were determined by size exclusion chromatography using two different methods: (1) the Akta Explorer 900 with Superose-6 10/300 GL chromatography column (GE Healthcare, Uppsala, Sweden) and (2) the e2695 Separations Module (Waters Corporation, Milford, Mass.) with Bio-Sil 250 SEC HPLC column (Bio-Rad, Hercules, Calif.). For both these configurations, UV absorbance was detected at 280 nm.

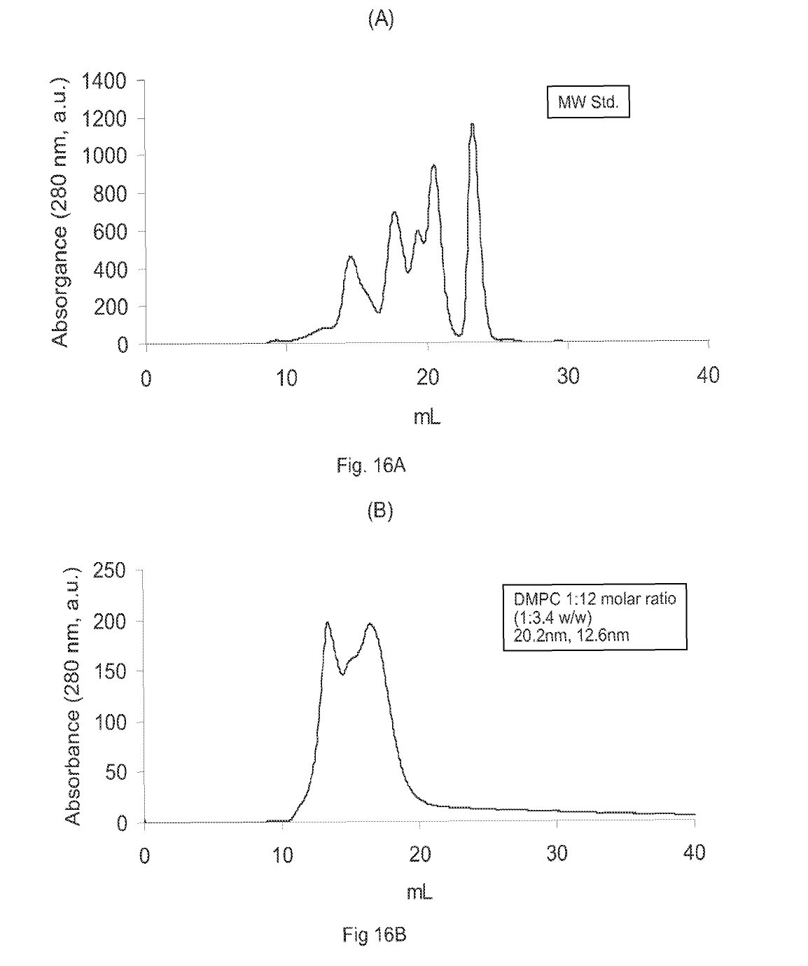

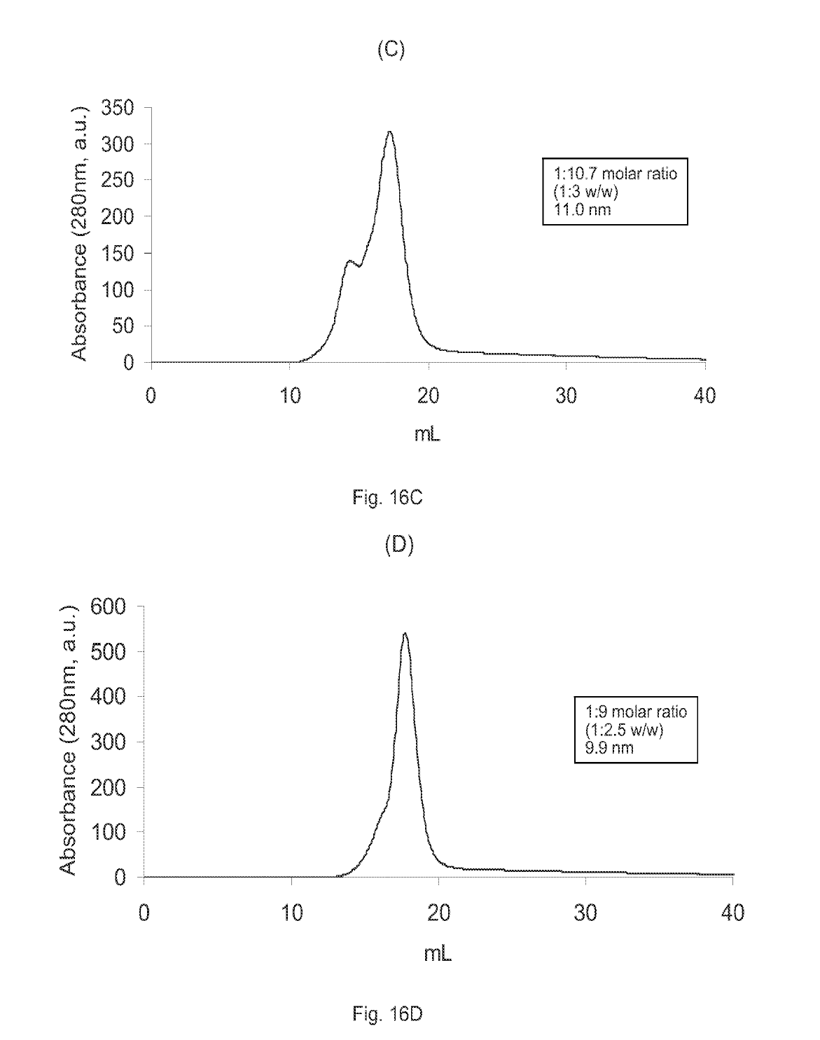

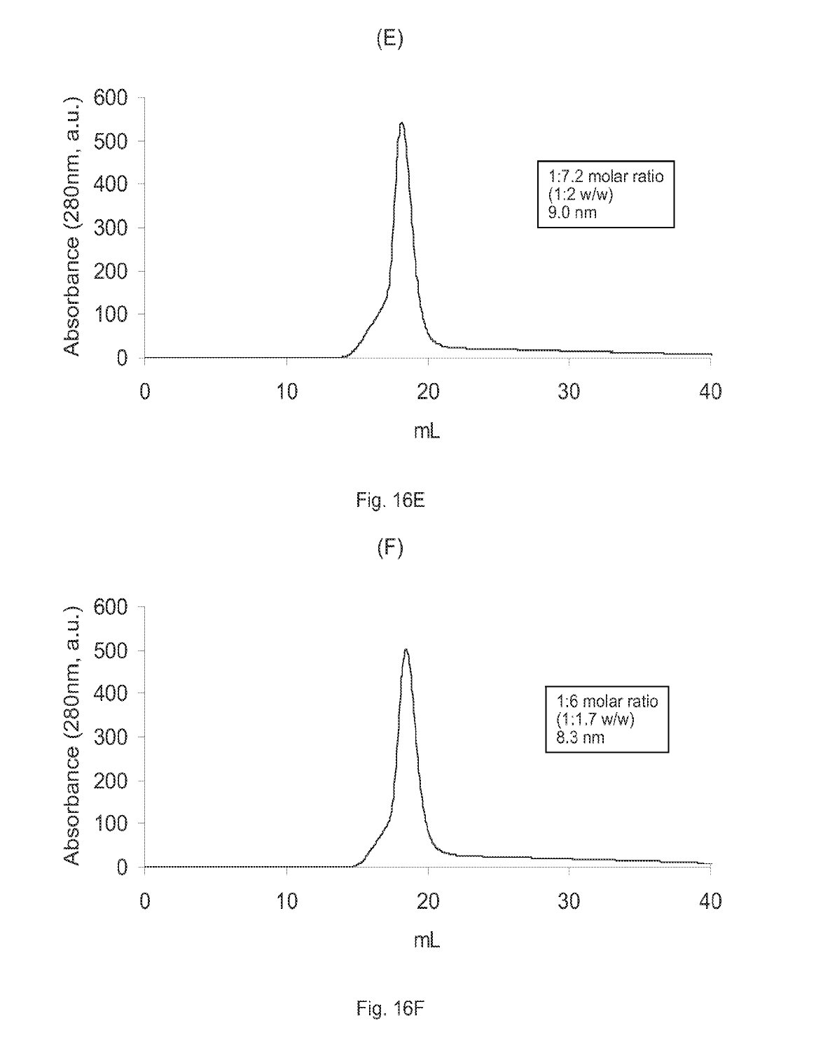

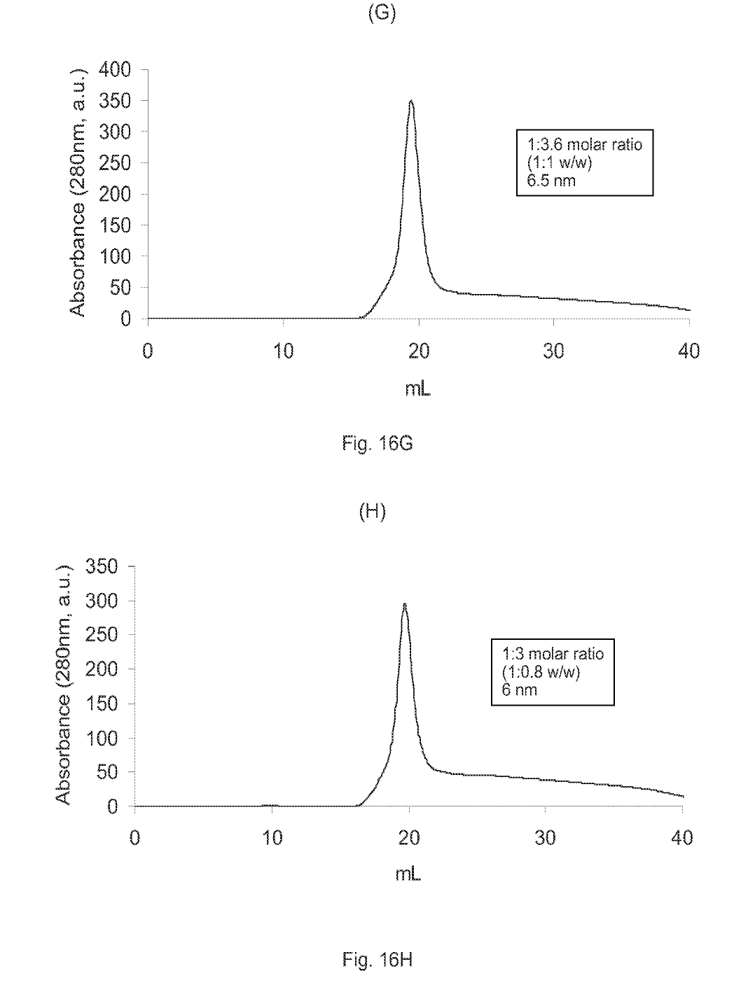

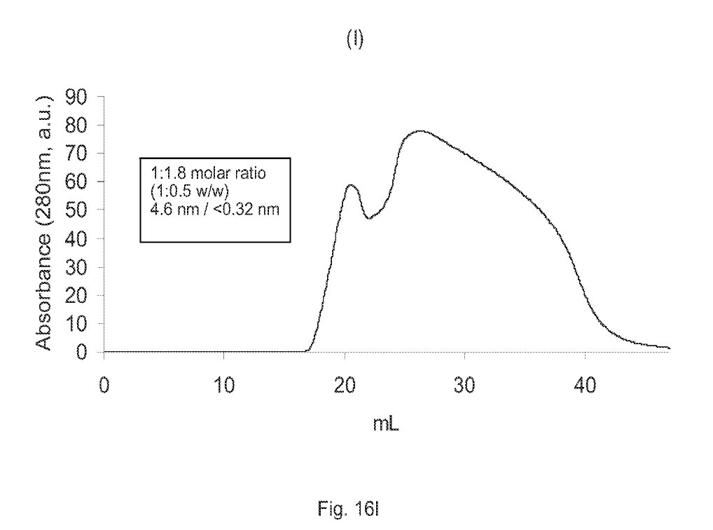

For the Akta Explorer chromatography method, particles were eluted with mobile phase (50 mM sodium phosphate and 150 mM sodium chloride) at a flow rate of 0.5 ml/min. Fractions were collected into 96-well deep block plates in increments of 2 mL. Protein standards of known molecular weight and Stokes diameter (Gel Filtration Standards, Bio-Rad) were run under these same conditions, and particle sizes were determined by comparing the retention times of the samples with those of the standard proteins. NLPP particles were formed at peptide:DMPC weight ratios of 1:3.4, 1:3, 1:2.5, 1:2, 1:1.7, 1:1, and 1:0.8, and hydrated in phosphate buffered saline to a concentration of 4 mg/mL peptide. The chromatogram for the gel filtration protein standards is also included for reference. FIG. 16 illustrates size exclusion chromatograms the NLPP particles, using the e2695 Separations Module method. Stokes diameters for particles prepared at peptide:DMPC weight ratios of 1:3.4, 1:3, 1:2.5, 1:2, 1:1.7, 1:1, and 1:0.8 were determined to be 20.2 nm, 11.0 nm, 9.9 nm, 9.0 nm, 8.3 nm, 6.5 nm, and 6.0 nm, respectively.

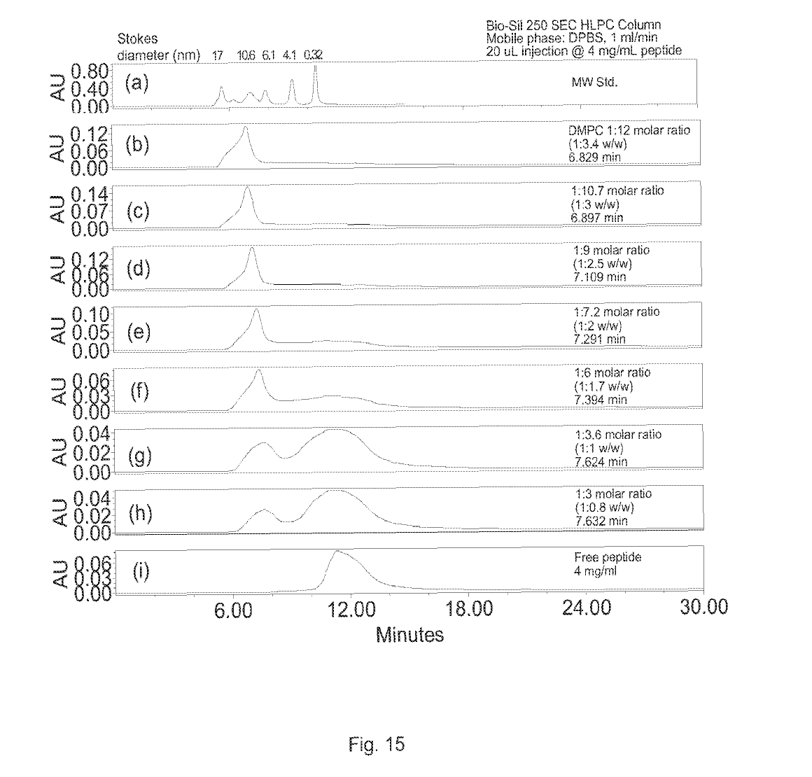

For the e2695 Separations Module method, particles were eluted with phosphate buffered saline at a flow rate of 1 ml/min. Protein standards of known molecular weight were run under these same conditions, and particle sizes were estimated by comparing the retention times of the samples with those of the standard proteins. FIG. 15 shows size exclusion chromatograms of NLPP particles, using the e2695 Separations Module method. NLPP particles were formed at peptide:DMPC weight ratios of 1:3.4, 1:3, 1:2.5, 1:2, 1:1.7, 1:1, and 1:0.8, and hydrated in phosphate buffered saline to a concentration of 4 mg/mL peptide. The chromatograms for the free peptide and the gel filtration protein standards are also included for reference. Particles prepared at peptide:DMPC weight ratios of 1:3.4, 1:3, 1:2.5, 1:2, 1:1.7, 1:1, and 1:0.8 were eluted from the column at 6.829 min, 6.897 min, 7.109 min, 7.291 min, 7.394 min, 7.624 min, and 7.632 min, respectively.

For particles containing SMIP, a stock solution of the SMIP compound was prepared in methylene chloride at a concentration of 5 ug/mL and an appropriate volume of the stock solution was combined in glass dram vials with the lipid-peptide methanol solutions described above (peptide:DMPC weight ratio of 2.5:1). The model SMIP used in these experiments was 2-(4-(isopentyloxy)-2-methylphenethyl)-8-methylbenzo[f][1,7]naphthyridin-5-amine.

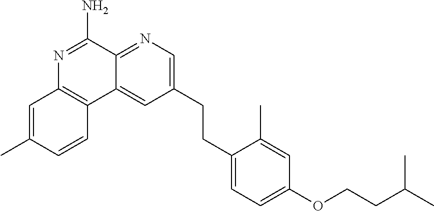

Thin films comprising lipid, peptide and SMIP were cast by solvent rotary evaporation. The films were hydrated in 10 mL sterile phosphate buffered saline to achieve final peptide and SMIP concentrations of 4 mg/mL and 250 ug/mL, respectively. The resulting particle suspension was then transferred to the upper reservoir of the Amicon Ultra 15 (10,000 MWCO; Millipore, Billerica, Mass.) centrifugal filter device for concentration. The device was centrifuged at 2,000 g for 15 minutes, the retentate and filtrate were recovered and subsequently analyzed for SMIP concentration.

SMIP concentrations in the fractions were measured by ultra-performance liquid chromatography (HPLC) using an Acquity HPLC BEH C8 column (2.1×100 mm; Waters Corporation Milford, Mass.). The mobile phase was a 0-100% water-acetonitrile gradient, and detection was by ultraviolet absorbance at 325 nm. Standards of known SMIP concentrations were run using the same method.

The SMIP concentration in the retentate volume was 1.217 mg/ml, and no detectable levels of SMIP were observed to be present in the filtrate; phospholipids in the retentate were determined to be 1.217 mg/mL.

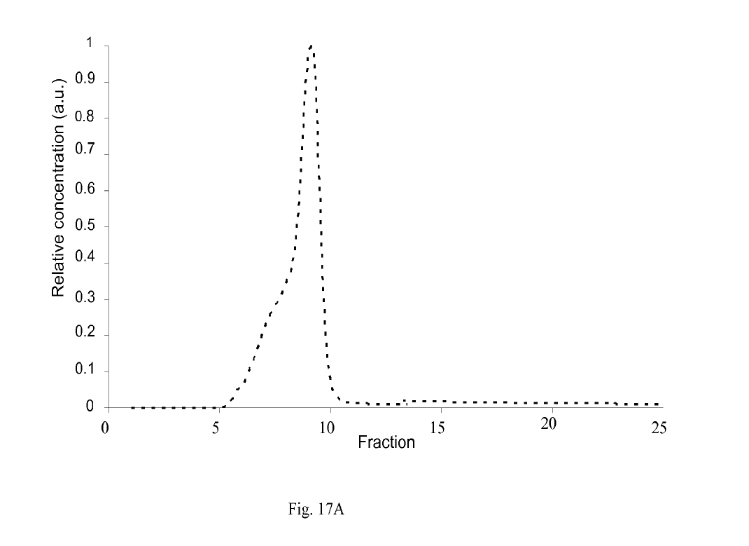

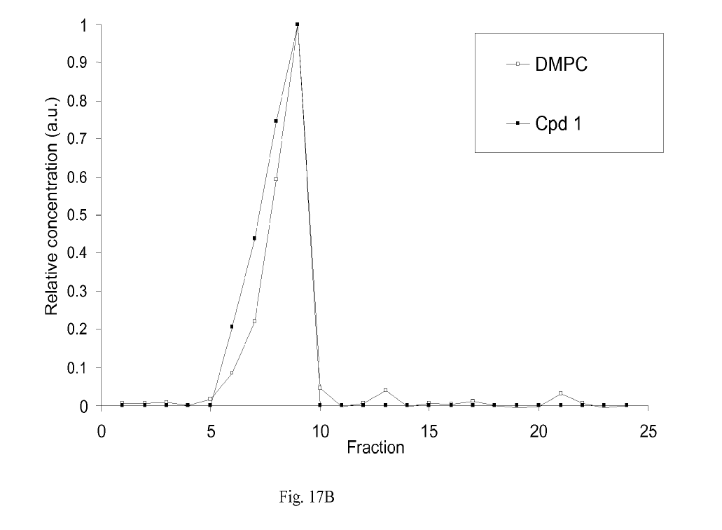

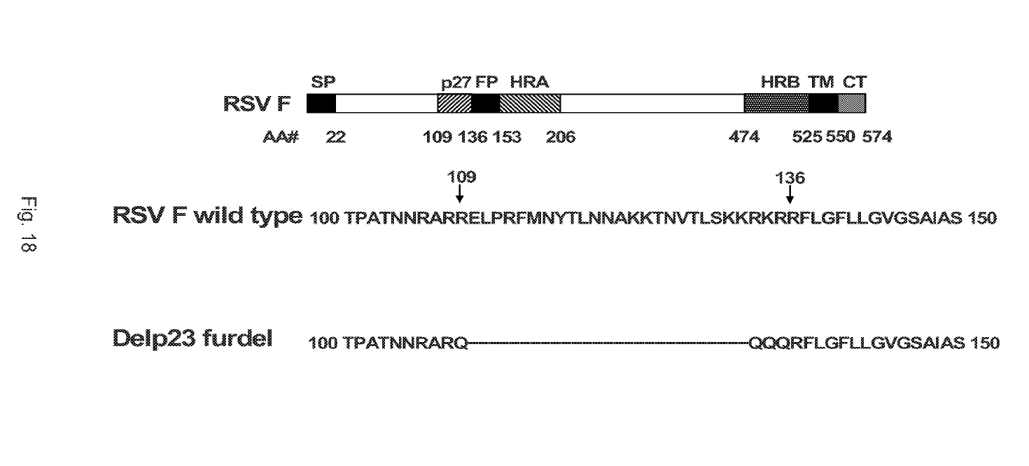

Size exclusion chromatography fractions were collected (using the Akta 900 Explorer method described above), and subsequently analyzed for lipid and SMIP content. The peak fractions were those numbered 5-9. FIG. 17 shows the following: (a) Size exclusion chromatogram for NLPP particles at a lipid:DMPC ratio of 1:2.5 and containing the SMIP at a concentration of 1.2 mg/mL. The chromatogram peak was collected in fractions 5-9. (b) Size exclusion chromatography fraction analysis for SMIP and phospholipid content. The peak concentrations for both phospholipids and SMIP appear in the same fractions, indicating the co-elution of the peptide, lipid, and SMIP.

Phospholipids were quantitated using a colorimetric Phospholipids C Assay reagent kit (Wake Diagnostics, Japan) and used according to the manufacturer's instructions without further modification. The phospholipids concentrations in sequential order of fractions 5-9 were: 0.02, 0.1, 0.28, 0.76, and 1.3 mg/mL. No phospholipids (<0.001 mg/mL) were detected in any fractions outside this range. SMIP concentrations were determined using the ultra-performance liquid chromatography method described above. The SMIP concentrations in sequential order of fractions 6-9 were: 3.9, 8.3, 14.1, and 18.9 ug/mL, respectively. No detectable level of SMIP was found in fraction 5, nor in any other fractions.

Similar procedures were employed to form (a) particles loaded with 2-(2,4-dimethylphenethyl)benzo[f][1,7]naphthyridin-5-amine, using particles formed using DMPC (3:1 w:w DMPC:peptide) with 4 mg/ml peptide concentration and SMIP incorporation up to 68 ug/ml and (b) particles loaded with imiquimod, using particles formed using DMPC (3:1 w:w DMPC:peptide) with 4 mg/ml peptide concentration and SMIP incorporation up to 60 ug/ml. For particles loaded with resiquimod, the extent of incorporation was difficult to determine.

2-(4-(Isopentyloxy)-2-methylphenethyl)-8-methylbenzo[f][1,7]naphthyridin-5-amine, also referred to herein as Compound 1 (Cpd 1), was prepared as follows:

Step 1: tert-butyl 2-bromo-5-methylphenylcarbamate

To a solution of 2-bromo-5-methylaniline (1.0 eq.) in tetrahydrofuran (0.2 M) at 0° C. under N₂ atmosphere was added dropwise 1M NaHMDS (2.5 eq.). The reaction was stirred for 15 minutes at 0° C., and a solution of di-tert-butyl dicarbonate in tetrahydrofuran was added. The reaction was warmed to room temperature overnight. The solvent was evaporated, and the resulting residue was quenched with 0.1N HCl aqueous solution. The aqueous suspension was extracted twice with ethyl acetate. The combined organic layers were washed with brine, dried over anhydrous MgSO₄, and concentrated en vacuo. The crude material was purified by flash chromatography on a COMBIFLASH® system (ISCO) using 0-5% ethyl acetate in hexane to give product as light yellow oil.

Step 2: tert-butyl 5-methyl-2-(4,4,5,5-tetramethyl-1,3,2-dioxaborolan-2-yl)phenylcarbamate

Tert-butyl 2-bromo-5-methylphenylcarbamate (1.0 eq.), 4,4,4′,4′,5,5,5′,5′-octamethyl-2,2′-bi(1,3,2-dioxaborolane) (1.5 eq.), dichloro[1,1′-bis(diphenylphosphino)ferrocene]palladium (II) (5%), and sodium acetate (4.5 eq.) were mixed in dioxane (0.2 M) under N₂ atmosphere. The reaction was heated to 100° C. and stirred overnight. The resulting suspension was cooled to ambient temperature, diluted with ether, filtered through celite, and the filtrate was concentrated en vacuo. The crude material was purified by flash chromatography on a COMBIFLASH® system (ISCO) using 0-8% ether in hexane to give tert-butyl 5-methyl-2-(4,4,5,5-tetramethyl-1,3,2-dioxaborolan-2-yl)phenylcarbamate.

Step 3: 3-chloro-5((4-methoxy-2-methylphenyl)ethynyl)picolinonitrile

To a round bottom flask capped with septa was added 1-ethynyl-4-methoxy-2-methylbenzene (1.1 eq), 3,5-dichloropicolinonitrile (1 eq.), triethylamine (5 eq.), and anhydrous DMF (0.2 M). Vacuumed and nitrogen flushed for three times. CuI (0.05 eq.) and bis(triphenylphosphine)dichloro-palladium(II) (0.05 eq) were added. The septum was replaced with a refluxing condenser and the flask was heated at 60° C. overnight under nitrogen atmosphere. Upon completion of the reaction as monitored by TLC, the content of the flask was loaded onto a large silica gel column pretreated with hexanes. Flash chromatography (silica gel, hexanes:EtOAc (1:4%)) afforded the product 5#2-methyl-4-methoxyphenyl)ethynyl)-3-chloropicolinonitrile.

Step 4: 2-((4-methoxy-2-methylphenyl)ethynyl)-8-methylbenzo[f][1,7]naphthyridin-5-amine

To a round bottom flask with refluxing condenser were added 5-((2-methyl-4-methoxyphenyl)ethynyl)-3-chloropicolinonitrile (1 eq.), tert-butyl 2-(4,4,5,5-tetramethyl-1,3,2-dioxaborolan-2-yl)phenylcarbamate (1.25 eq.), K₃PO₄ (2 eq.), tris(dibenzylideneacetone)dipalladium(0) (0.05 eq.), and 2-Dicyclohexylphosphino-2′,6′-dimethoxybiphenyl (0.1 eq.). n-Butanol and water (5:2, 0.2 M) were added, and the content were degassed (vacuum followed by nitrogen flush) for three times. The reaction mixture was stirred vigorously under nitrogen at 100° C. overnight in an oil bath. The contents were cooled down and were taken up in 200 mL of water followed by extraction with methylene chloride. Combined organic layers were dried (Na₂SO₄) and concentrated. Flash chromatography (silica gel, 0-50% EtOAc in CH₂Cl₂) afforded the product, 2((4-Methoxy-2-methylphenyl)ethynyl)-8-methylbenzo[f][1,7]naphthyridin-5-amine

Step 5: 2-(4-methoxy-2-methylphenethyl)-8-methylbenzo[f][1,7]naphthyridin-5-amine

To a round bottom flask was added 2#4-methoxy-2-methylphenyl)ethynyl)-8-methylbenzo[f][1,7]naphthyridin-5-amine (1 eq.) with a stirring bar. Ethanol and methylene chloride (1:2, 0.2 M) were added, followed by palladium in carbon (activated powder, wet, 10% on carbon, 0.1 eq.). The contents were vacuumed followed by hydrogen flush for three times. The reaction mixture was stirred vigorously under hydrogen balloon at room temperature overnight. Afterwards the reaction mixture was filtered through a celite pad, and the celite pad was washed subsequently with methylene chloride and EtOAc until the filtrate had no UV absorption. Combined organic washes were concentrated. Flash chromatography (silica gel, 0-50% EtOAc in CH₂Cl₂) afforded the product, 2-(4-Methoxy-2-methylphenethyl)-8-methylbenzo[f][1,7]naphthyridin-5-amine, as a yellow solid.

Step 6: 4-(2-(5-amino-8-methylbenzo[f][1,7]naphthyridin-2-yl)ethyl)-3-methylphenol

To a stirred solution of 2-(4-methoxy-2-methylphenethyl)-8-methylbenzo[f][1,7]naphthyridin-5-amine in methylene chloride (0.2 M) in an ice-water bath was added 1 N solution of BBr₃ (2 eq) in CH₂Cl₂ in a drop-wise fashion. In 30 minutes the reaction was quenched with methanol and was concentrated en vaccuo to obtain a crude residue. The crude material was purified by flash chromatography on a COMBIFLASH® system (ISCO) using 0-20% methanol in dichloromethane to give 4-(2-(5-amino-8-methylbenzo[f][1,7]naphthyridin-2-yl)ethyl)-3-methylphenol as a white solid.

Step 7: 2-(4-(isopentyloxy)-2-methylphenethyl)-8-methylbenzo[f][1,7]naphthyridin-5-amine

To a solution of 4-(2-(5-amino-8-methylbenzo[f][1,7]naphthyridin-2-yl)ethyl)-3-methylphenol (1.0 equiv.) in dimethylformamide (0.10 M) was added anhydrous potassium carbonate (1.5 equiv.) followed by 1-bromo-3-methylbutane (1.2 equiv.). The resulting mixture was allowed to stir for 18 hours at 100° C. After cooling to ambient temperature, the mixture was diluted with ethyl acetate and water. The biphasic layers were separated and the aqueous layer was washed twice with ethyl acetate. The combined organic layers were dried over anhydrous Na₂SO₄ and the volatiles were removed in vacuo. The resulting residue was purified by a COMBIFLASH® system (ISCO) using 0-60% ethyl acetate in hexanes to provide 2-(4-(isopentyloxy)-2-methylphenethyl)-8-methylbenzo[f][1,7]naphthyridin-5-amine ¹H NMR (Acetone-d₆): δ 8.75 (s, 1H), 8.72 (s, 1H), 8.29 (d, 1H), 7.43 (s, 1H), 7.17 (D, 1H), 7.10 (d, 1H), 6.76 (d, 1H), 6.68 (d, 1H), 6.56 (br, 2H), 4.00 (t, 2H), 3.17 (t, 2H), 3.07 (t, 2H), 2.48 (s, 3H), 1.76-1.91 (m, 1H), 1.60-1.71 (m, 2H), 0.96 (s, 6H). LRMS [M+H]=414.2.

2-(2,4-dimethylphenethyl)benzo[f][1,7]naphthyridin-5-amine was prepared as follows:

Step 1: ((2,4-dimethylphenyl)ethynyl)triethylsilane

To a scintillation vial was added 1-iodo-2,4-dimethylbenzene (commercially available) (1.1 eq.), triethyl(ethynyl)silane (1 eq.), triethylamine (5 eq.), and anhydrous DMF (0.2 M). Vacuumed and nitrogen flushed for three times. CuI (0.1 eq.) and bis(triphenylphosphine)dichloro-palladium(II) (0.1 eq) were added. The vial was sealed and heated at 60° C. overnight. Upon completion of the reaction as monitored by TLC, the content of the vial was loaded onto a silica gel column pretreated with hexanes. Column was washed with hexanes and diethylether until all eluents containing product were collected. Carefully distill off hexanes and ether using rotary evaporator with minim heating afforded product ((2,4-dimethylphenyl)ethynyl)triethylsilane, which was carried directly on to the next step.

Step 2: 1-ethynyl-2,4-dimethylbenzene

To a stirred solution of ((2,4-dimethylphenyl)ethynyl)triethylsilane (from the previous step) in THF (0.2 M) cooled at 0° C. was treated with a solution (0.5 eq.) of tetrabutylammonium fluoride in a dropwise fashion. The reaction mixture turned black and was continued to stir for 30 minutes before warming up to rt. TLC showed full conversion. The reaction was quenched with water and was extracted with diethylether. Combined organic layers were dried over anhydrous Na₂SO₄ and concentrated using rotary evaporator with minim heating. Chromatography (silica gel, diethylether) afforded the product 1-ethynyl-2,4-dimethylbenzene.

Step 3: 3-chloro-5((2,4-dimethylphenyl)ethynyl)picolinonitrile

To a round bottom flask capped with septa was added 1-ethynyl-2,4-dimethylbenzene (from the previous step) (1.1 eq), 3,5-dichloropicolinonitrile (1 eq.), triethylamine (5 eq.), and anhydrous DMF (0.2 M). Vacuumed and nitrogen flushed for three times. CuI (0.05 eq.) and bis(triphenylphosphine)dichloro-palladium(II) (0.05 eq) were added. The septum was replaced with a refluxing condenser and the flask was heated at 60° C. overnight under nitrogen atmosphere. Upon completion of the reaction as monitored by TLC, the content of the flask was loaded onto a large silica gel column pretreated with hexanes. Flash chromatography (silica gel, hexanes:EtOAc (1:4%)) afforded the product 3-chloro-5-((2,4-dimethylphenyl)ethynyl)picolinonitrile.

Step 4: 2-((2,4-dimethylphenyl)ethynyl)benzo[f][1,7]naphthyridin-5-amine

To a round bottom flask with refluxing condenser were added 3-chloro-5-((2,4-dimethylphenyl)ethynyl)-picolinonitrile (from the previous step) (1 eq.), tert-butyl 2-(4,4,5,5-tetramethyl-1,3,2-dioxaborolan-2-yl)phenylcarbamate (1.25 eq.), K₃PO₄ (2 eq.), tris(dibenzylideneacetone)dipalladium(0) (0.05 eq.), and 2-dicyclohexylphosphino-2′,6′-dimethoxybiphenyl (0.1 eq.). n-Butanol and water (5:2, 0.2 M) were added, and the content were degassed (vacuum followed by nitrogen flush) for three times. The reaction mixture was stirred vigorously under nitrogen at 100° C. overnight in an oil bath. The content was cooled down and were taken up in 200 mL of water followed by extraction with methylene chloride. Combined organic layers were dried (Na₂SO₄) and concentrated. Flash chromatography (silica gel, 0-50% EtOAc in CH₂Cl₂) afforded the product 2-((2,4-dimethylphenyl)ethynyl)benzo[f][1,7]naphthyridin-5-amine

Step 5: 2-(2,4-dimethylphenethyl)benzo[f]-[1,7]naphthyridin-5-amine

To a round bottom flask was added 2-((2,4-dimethylphenyl)ethynyl)benzo[f][1,7]naphthyridin-5-amine (from the previous step) (1 eq.) with a stirring bar. Ethanol and methylene chloride (1:2, 0.2 M) were added, followed by palladium in carbon (activated powder, wet, 10% on carbon, 0.1 eq.). The content was vacuumed followed by hydrogen flush for three times. The reaction mixture was stirred vigorously under hydrogen balloon at room temperature overnight. Afterwards the reaction mixture was filtered through a celite pad, and the celite pad was washed subsequently with methylene chloride and EtOAc until the filtrate had no UV absorption. Combined organic washes were concentrated. Flash chromatography (silica gel, 0-50% EtOAc in CH₂Cl₂) afforded the product 2-(2,4-Dimethylphenethyl)benzo[f][1,7]naphthyridin-5-amine ¹H NMR (CDCl₃): δ 8.60 (d, 1H), 8.33 (d, 1H), 8.14 (d, 1H), 7.67 (d, 1H), 7.54 (t, 1H), 7.31 (t, 1H), 6.96-6.86 (m, 3H), 6.29 (bs, 2H), 3.04-3.10 (dd, 2H), 2.97-2.91 (dd, 2H), 2.24 (s, 3H), 2.20 (s, 3H). LRMS [M+H]=328.2.

### Example 15

### In Situ Incorporation of Influenza M2e-TM (M2 Ectodomain Transmembrane Protein) in Particles Using Cell-Free Protein Synthesis: SDS-PAGE Characterization

The in-situ incorporation of the model influenza protein M2e-TM within the lipid bilayer of the particles was performed by using the particles in conjunction with the S30 Protein Expression kit (Promega, Madison, Wis.). This reagent kit contains the S30 Premix Plus and T7 S30 Extract reagents, and these components of the expression kit were used without further modification. To perform the protein synthesis reaction, the following components were combined in a 1.7 mL eppendorf tube: 1 ug of plasmid DNA encoding the influenza M2e-TM; 10 uL of various particle formulations (e.g., at a concentration of 4 mg/ml peptide); 20 uL of S30 Premix Plus reagent; 18 uL of T7 S30 Extract, and an optional addition of nuclease-free water, if necessary, to reach a total 50 uL reaction volume. The samples were incubated at 37° C. with vigorous shaking for 2 hours.

The samples were retrieved and placed on ice for 10 minutes. A 20 uL aliquot of the total reaction mix was transferred to a clean eppendorf microcentrifuge tube and reserved for denaturing page electrophoresis, and the remaining mixture was placed in a microcentrifuge at 16,000 g for 10 minutes to separate and recover the soluble portion of the protein synthesis reaction.

To process the samples for electrophoresis, 5 uL of each sample were precipitated in acetone and resuspended in a final volume of 20 uL of LDS Sample buffer (Invitrogen, Carlsbad, Calif.). These treated samples were heated at 75° C. for 10 minutes, and 10 uL of each of the treated samples were loaded on a NUPAGE 12% Bis-Tris electrophoresis gel (Invitrogen). Samples were electrophoresed at 175 volts for 1 hour. Gels were treated with the Colloidal Blue Staining Kit (Invitrogen) for protein band visualization.

SDS-PAGE analysis of Influenza M2E-TM protein expression in cell-free synthesis reactions in combination with NLPP was based on the following: Lane (1) denotes the negative control reaction, where no plasmid DNA for M2E-TM was included in the reaction mix, and shows background levels of proteins endogenous to the protein expression kit. Lanes 2-7 correspond to cell-free synthesis reactions in the presence of the following NLPP particle formulations, respectively: (2) 1:1 peptide:DOPC; (3) 1:1.5 peptide:DOPC; (4) 1:0.5 peptide:DMPC; (5) 1:1 peptide:DMPC; (6) 1:1.6 peptide:DMPC; (7) 1:2 peptide:DMPC. The expected molecular weight of the influenza M2E-TM protein is approximately 9 kD. Protein bands corresponding to the M2E-TM protein of interest appeared in lane numbers 2-7 in the total reaction lanes, suggesting that NLPP particles do not inhibit protein synthesis under these conditions. In additional gel lanes corresponding to the soluble protein fractions, the expected M2E-TM protein band was not apparent, indicating that any protein expression may be below the detection limit of the protein visualization technique.

### Example 16

### In Situ Incorporation of Bacteriorhodopisin in Particles Using Cell-Free Protein Synthesis: Western Blot Characterization

The in-situ incorporation of bacteriorhodopisin within the lipid bilayer of the particles was performed by including NLPP in cell-free synthesis reactions using the MembraneMax Protein Expression kit (Invitrogen). Components of the expression kit were used without further modification. To perform the protein synthesis reaction, the following components were combined in a 1.7 mL eppendorf tube: 20 uL of slyD-extract, 20 uL of IVPS reaction buffer; 1.25 uL of 50 mM amino acids mix; 1 uL of 75 mM methionine; 1 uL of T7 enzyme, 4.75 uL of NLPP particle suspension (when indicated); 2 uL of MembraneMax reagent (when indicated); and an optional addition of nuclease-free water, if necessary, to reach a total 50 uL reaction volume. The samples were incubated at 37° C. with vigorous shaking for 30 minutes. After the 30 minutes of incubation, 50 uL of a feed buffer was added to the reaction mixture, and the tubes were returned to 37° C. incubation for an additional 90 minutes. This feed buffer was comprised of 25 uL of 2×IVPS Feed Buffer, 1.25 uL amino acids, 1 uL 75 mM methionine; and 22.25 uL of nuclease-free water.

The protein expression reaction tubes were then retrieved and placed on ice for 10 minutes. A 20 uL aliquot of the total reaction mix was transferred to a clean eppendorf microcentrifuge tube and reserved for denaturing page electrophoresis, and the remaining mixture was placed in a microcentrifuge at 16,000 g for 10 minutes to separate and recover the soluble portion of the protein synthesis reaction.

To process the samples for electrophoresis, 5 uL of each sample were precipitated in acetone and resuspended in a final volume of 20 uL of LDS Sample buffer (Invitrogen, Carlsbad, Calif.). These treated samples were heated at 75° C. for 10 minutes, and 10 uL of each of the treated samples was loaded on a NUPAGE 12% Bis-Tris electrophoresis gel (Invitrogen). Following electrophoresis, proteins from the gel were transferred to nitrocellulose membranes and probed with anti-his6 mouse IgG (1:500 dilution, Invitrogen) primary antibody and Alexa-680 conjugated goat anti-mouse IgG secondary antibody (1:10,000 dilution, Molecular Probes, Eugene, Oreg.). Protein band visualization was performed using the Odyssey Infrared Imaging System (LiCor Biosciences, Lincoln, Nebr.).

Western Blot detection of bacteriorhodopisin expression in cell free synthesis reactions in combination with NLPP was based on the following: Lane (0) Molecular weight standard markers; (1) No plasmid negative control; (2) Positive control: Membrane Max reagent, total reaction mixture; (3) Positive control: Membrane Max reagent, soluble fraction; (4) Negative Control: no Membrane Max reagent, total reaction mixture; (5) Negative Control: no Membrane Max reagent, soluble fraction; (6) 1:1 peptide:POPC particles, total reaction mixture; (7) 1:1 peptide:POPC particles, soluble fraction; (8) 1:2 peptide:POPC particles, total reaction mixture; (9) 1:2 peptide:POPC particles, soluble fraction. The expected molecular weight of the bacteriorhodopisin protein is approximately 24 kD. The protein bands above 24 kDa indicated endogenous anti-his6 cross-reactive proteins present in the expression kit and are considered background. In the absence of the MembraneMax reagent (lanes 4 and 5), a protein band corresponding to bacteriorhodopisin appeared in the total reaction mixture, but not in the soluble fraction. Protein bands corresponding to bacteriorhodopisin appeared in lanes 7 and 9 (lane 9 appeared very faintly), suggesting that the presence of the peptide:POPC particles enable the solubilization of bacteriorhodopisin.

1. A composition, comprising: (a) particles that comprise (i) an amphipathic peptide that comprises less than 30 amino acids and (ii) a lipid and (b) at least one immunogenic species associated with said particles.

2. The composition according to claim 1, wherein the particles are disc-shaped particles with a lipid core.

3. The composition according to claim 1, wherein said amphipathic peptide comprises 20 or less amino acids.

4. The composition according to claim 1, wherein said amphipathic peptide forms a class A amphipathic alpha helix.

5. The composition according to claim 1, wherein said amphipathic peptide mimics properties of apolipoprotein A1.

6. The composition according to claim 5, wherein said amphipathic peptide shows no sequence homology to apolipoprotein A1.

7. The composition according to claim 1, wherein the amphipathic peptide comprises an amino acid sequence selected from the following: (i) DWLKAFYDKVAEKLKEAFLA (Seq. ID No. 1), (ii) ELLEKWKEALAALAEKLK (Seq. ID No. 2), (iii) FWLKAFYDKVAEKLKEAF (Seq. ID No. 3), (iv) DWLKAFYDKVAEKLKEAFRLTRKRGLKLA (Seq. ID No. 4), and (v) DWLKAFYDKVAEKLKEAF (Seq. ID No. 5).

8. The composition according to claim 1, wherein the lipid is a phospholipid.

9. The composition according to claim 8, wherein the phospholipid is a zwitterionic phospholipid.

10. The composition according to claim 9, wherein zwitterionic phospholipid comprises a polar phosphatidylcholine head group.

11. The composition according to claim 9, wherein the phospholipid comprises one or more alkyl or alkenyl radicals of 12-22 carbons in length and containing 0 to 3 double bonds.

12. The composition according to claim 1, wherein the immunogenic species is an antigen.

13. The composition according to claim 12, wherein the antigen comprises a covalently or non-covalently attached lipophilic anchor.

14. The composition according to claim 13, wherein the lipophilic anchor is a native membrane anchoring region of the antigen.

15. The composition according to claim 13, wherein the lipophilic anchor is attached via a cleavable linker.

16. The composition according to claim 12, wherein the antigen is influenza hemagglutinin (HA).

17. The composition according to claim 12, wherein the antigen is a polynucleotide that expresses an immunogenic protein.

18. The composition according to claim 17, wherein the composition further comprises a cationic lipid.

19. The composition according to claim 1, wherein the immunogenic species is an immunological adjuvant.

20. The composition according to claim 19, wherein the immunological adjuvant is selected from bacterial lipopolysaccharides, bacterial lipoproteins, antimicrobial peptides, saponins, lipoteichoic acid, squalene, immunostimulatory oligonucleotides, single-stranded RNA, synthetic phospholipids, MF59, E6020, IC31, lipopeptides, imidazoquinoline compounds, and benzonaphthyridine compounds.

21. The composition according to claim 1, wherein the particles are less than 50 nm in width.

22. The composition according to claim 1, wherein the particles range from 50 nm to 10,000 nm in width.

23. The composition according to claim 22, wherein the particles are aggregates of smaller particles.

24. The composition according to claim 22, wherein the immunogenic species comprise DNA or RNA, and wherein composition further comprises a cationic lipid as a capturing agent.

25. The composition according to claim 1, wherein the composition comprises a targeting ligand for targeting antigen presenting cells.

26. The composition according to claim 1, comprising one or more supplemental components selected from liquid vehicles, agents for adjusting tonicity, agents for adjusting pH, surfactants and cryoprotective agents.

27. The composition according to claim 1, wherein said composition is a lyophilized composition.

28. The composition according to claim 1, wherein said composition is sterile filtered.

29. A method of raising an immune response in a vertebrate subject comprising delivering the immunogenic composition of claim 1 to said vertebrate subject.

30. A method of forming an immunogenic composition, comprising synthesizing or modifying an immunogenic species in the presence of particles that comprise (i) an amphipathic peptide that comprises less than 30 amino acids and (ii) a lipid, wherein said synthesized or modified immunogenic species becomes associated with said particles as a result of said synthesis or modification step.

31. The method of claim 30, wherein said immunogenic species is a protein that is synthesized in the presence of said particles.

32. The method of claim 30, wherein said immunogenic species is a protein that is cleaved in the presence of said particles.
